(19) United States
(12) Patent Application Publication
(10) Pub. No.: US 2025/0304990 A1
(43) Pub. Date: Oct. 2, 2025

(54) HERBICIDE RESISTANCE

(71) Applicant: SYNGENTA CROP PROTECTION AG, Basel (CH)

(72) Inventors: Richard Paul DALE, Bracknell, Berkshire (GB); Rachael Elizabeth BLAIN, Bracknell, Berkshire (GB); Marta Andreia Horta SIMOES, Bracknell, Berkshire (GB); Michael Phillip LANGFORD, Bracknell, Berkshire (GB)

(73) Assignee: SYNGENTA CROP PROTECTION AG, Basel (CH)

(21) Appl. No.: 18/709,433

(22) PCT Filed: Nov. 10, 2022
(86) PCT No.: PCT/EP2022/081497

§ 371 Date: May 10, 2024

(30) Foreign Application Priority Data
Nov. 12, 2021 (GB) 2116307.6

(51) Int. Cl. C12N 15/82; A01N 43/58; A01N 43/76; A01P 13/00; C12N 9/10; C12N 9/22; C12N 15/11

(52) U.S. Cl. CPC C12N 15/8274; A01N 43/58; A01N 43/76; A01P 13/00; C12N 9/1085; C12N 9/222; C12N 15/111; C12N 15/8201; C12N 2310/20; C12Y 205/01

(57) ABSTRACT

Screening assays are used to identify mutant homogentisate solanesyl transferase (HST) enzymes which are at least partially resistant to HST-inhibiting herbicides. Nucleic acids encoding the mutant HST enzyme proteins are made available and useful in modifying plants and plant parts so that they can be made herbicide resistant. Crop plants which express the mutant HST enzymes can be grown in the field and herbicides used to controlling unwanted other vegetation. The homogentisate solanesyltransferase (HST) enzyme or an active fragment thereof comprises the amino acid sequence motif: F[V/M]TX[F/Y] (SEQ ID NO: 1), wherein X is any amino acid; and wherein one or more of the amino acid residues of the motif are mutated.

### CROSS-REFERENCE TO RELATED APPLICATIONS

This application is a U.S. National Stage Application, filed under 35 U.S.C. § 371, of International Application No. PCT/EP2022/081497, filed Nov. 10, 2022, which claims priority to GB 2116307.6, filed Nov. 12, 2021, the entire contents of which are incorporated by reference herein.

### SEQUENCE LISTING

A Sequence Listing in XML format, submitted under 37 C.F.R. § 1.834(c)(1), entitled “115479-000863_Sequence-Listing.xml”, 121 kilobytes in size, generated on Nov. 28, 2024 is provided. This Sequence Listing is hereby incorporated by reference into the specification for its disclosures.

### FIELD OF THE INVENTION

The invention relates to mutant homogentisate solanesyl transferase (HST) enzymes that are at least partially resistant to HST-inhibiting herbicides. The invention includes nucleic acids and proteins encoding such mutants. The invention also relates to plants and parts thereof including such mutants and methods of growing and propagating such plants. The invention also relates to methods of improving plant growth and controlling unwanted vegetation using such mutant plants and parts thereof.

### BACKGROUND

The present invention relates to the production of plants that are resistant to herbicides that inhibit homogentisate solanesyl transferase (HST) enzyme (also sometimes referred to as homogentisate prenytransferase). HST is a prenyl tranferase that both decarboxylates homogentisate and also transfers to it the solanesyl group from solanesyl diphosphate and thus forms 2-methyl-6-solanesyl-1,4-benzoquinol (MSBQ), an intermediate along the biosynthetic pathway to plastoquinone. Plastoquinone-9 (PQ-9)2 is the major prenylated quinone in chloroplasts. In the thylakoid membrane, it mediates electron flow from photosystem II to the cytochrome b6f complex, and the redox state of the PQ-9 pool regulates the expression of a number of nuclear and plastidial genes as well as the activity of some plastidial enzymes. Moreover, PQ-9 is required as a cofactor for phytoene desaturation in carotenoid biosynthesis. Reflecting its central and unique role in higher plants, a defect in PQ-9 biosynthesis cannot be remedied by other plastidial prenylquinones such as phylloquinone or any of the structurally related intermediates of the vitamin E biosynthetic pathway. HST enzymes are membrane bound and the genes that encode them include a plastid targeting sequence. Methods for assaying HST have recently been disclosed.

Deletions of Val148 and Gly149 of Arabidopsis thaliana HST, as described in “Tian, Li, Dean DellaPenna, and Richard A. Dixon “The pds2 mutation is a lesion in the Arabidopsis homogentisate solanesyltransferase gene involved in plastoquinone biosynthesis.” Planta 226.4 (2007): 1067-1073 have been shown to lead to a loss or reduction in function of HST and a lack of α-tocopherol and plastiquinone in pds2 mutant plants.

Overexpression of HST in transgenic plants has been reported and said plants are said to exhibit slightly higher concentrations of α-tocopherol. Furthermore, it has previously been shown, for example in WO2010029311A2, that overexpression of HST in a transgenic plant provides tolerance to HST-inhibiting herbicides.

There has not yet been a link shown between any mutants of HST and increased resistance to herbicidal compounds.

### SUMMARY OF INVENTION

The present invention provides a homogentisate solanesyltransferase (HST) enzyme or an active fragment thereof, comprising the amino acid sequence motif: F[V/M]TX[F/Y] (SEQ ID NO: 1), wherein X is any amino acid; and wherein one or more of the amino acid residues of the motif are mutated.

In preferred forms, the HST enzyme of the invention is a mutant which retains substantially the wild type or usual levels of HST enzyme activity in the cell, whereby plastoquinone synthesis is substantially unaffected. The wild type HST enzyme is susceptible to inactivation by certain compounds which thereby have herbicidal effect. Without wishing to be bound by any particular theory, a mutation in the HST enzyme in accordance with the invention generally does not disrupt the HST enzyme activity, but rather the mutation disrupts the sensitivity of the HST enzyme to the compounds which have herbicidal effect on it in planta. In other words, the effect of mutation in HST in accordance with the invention increases the tolerance of the HST enzyme to the inhibitory effects of herbicidal compounds which would otherwise be the case in the absence of the mutation(s).

In preferred embodiments, X is a neutral amino acid or an hydrophobic amino acid; more preferably wherein X is an amino acid selected from leucine, methionine, phenylalanine, isoleucine, valine, tyrosine or cysteine.

The above motif consists of 5 amino acid residues and therefore 5 positions are identified hereinafter, numbered 1 to 5. F is position 1, [V/M] is position 2, T is position 3, X is position 4 and [F/Y] is position 5. In terms of mutations, one or more of the amino acid residues of the motif may be mutated, whether by deletion, insertion or substitution. Such deletion, insertion or substitution may involve one, two or three amino acids at a respective motif position. Some preferred mutations, whether alone or in any combination are as follows:
(a) substitution at position 1 of the motif; e.g. a non-conservative substitution; more preferably a substitution with an aliphatic amino acid;
(b) substitution at position 2 of the motif; e.g. a conservative substitution; more preferably a substitution with an aliphatic amino acid;
(c) substitution at position 3 of the motif, e.g. a non-conservative substitution; more preferably a substitution with an acidic amino acid;
(d) substitution at position 5 of the motif; e.g. a non-conservative substitution; more preferably a substitution with an aliphatic amino acid.

Other preferred mutations, whether alone or in any combination are as follows:
(a) mutation at position 1 of the motif to isoleucine, e.g. SEQ ID NO: 2;
(b) mutation at position 2 of the motif to alanine, e.g. as in SEQ ID NO: 3;
(c) mutation at position 3 of the motif to isoleucine, e.g. as in SEQ ID NO: 4;
(d) mutation at position 5 of the motif to isoleucine, e.g. as in (SEQ ID NO: 5);
(e) mutation at position 5 of the motif to lysine, e.g. as in (SEQ ID NO: 6).

In other embodiments of the HST enzyme or active fragments thereof, the motif is comprised within an amino acid sequence comprising any one of SEQ ID NOs: 13 to 21, or a sequence of at least 70% identity therewith. The range of possible variants of the HST enzyme may be narrower, for example the HST enzyme may comprise an amino acid sequence of at least 75% at least 80%, at least 85%, at least 90%, at least 95%, at least 98% or at least 99% sequence identity to an amino acid sequence according to SEQ ID NOs: 13 to 21.

An HST enzyme or active fragment thereof is preferably at least partially resistant to inhibition by an HST-inhibiting compound. Such at least partially resistant HST enzymes or active fragments may have at least 10-fold more resistance to inhibition by an HST-inhibiting compound, than a control or wild-type HST enzyme or corresponding active fragment not having the or each mutation; preferably wherein the control or wild-type HST has the amino acid sequence of any one of SEQ ID NO: 14, 16, 18, 20 or 21.

A mutant HST enzyme of the invention, or active fragment thereof, may have one or any combination of the amino acid substitutions F196I, V197A, T199N, F200I or F200L of SEQ ID NO:13, or one or any combination of the corresponding amino acid substitutions in any one of SEQ ID NOs: 14 to 16.

A mutant HST enzyme of the invention, or active fragment thereof, may have one or any combination of the amino acid substitutions F199I, V200A, T201N, F203I or F203L of SEQ ID NO:17, or one or any combination of the corresponding amino acid substitutions in SEQ ID NO: 18.

A mutant HST enzyme of the invention, or active fragment thereof, may have one or any combination of the amino acid substitutions F198I, V199A, T200N, F202I or F202L of SEQ ID NO:19, or one or any combination of the corresponding amino acid substitutions in any one of SEQ ID NOs: 20 to 21.

In independent aspect to the above, the invention also provides an HST enzyme comprising an amino acid sequence of SEQ ID NO: 14 or a sequence of at least 70% identity therewith, or a functional fragment thereof, and wherein the amino acid sequence of the enzyme or fragment has a mutation in at least one position selected from positions 276, 277, 278, 279 and 280 of SEQ ID NO:14, or positions corresponding thereto in any homologous or related HST enzyme sequence from the same or another species. Additionally, in this aspect of the invention, the amino acid sequence of SEQ ID NO: 14 which comprises the motif FVTLFA may have that motif modified or replaced so that SEQ ID NO: 14 comprises any one of the motifs as hereinbefore defined.

A mutant HST enzyme of any aspect of this invention, or active fragment thereof, may comprise an additional amino acid sequence; preferably wherein this additional amino acid sequence is transit peptide. The transit peptide may be naturally occurring or a modified sequence.

More particularly, a mutant HST enzyme of the invention may comprise an amino acid sequence which is at least 70% identical to a reference sequence selected from SEQ ID NOs: 22 to 69, or an active fragment thereof.

Further particular mutant HST enzymes of the invention may comprise or consist of an amino acid sequence selected from SEQ ID NO: 22 to 69, or an active fragment thereof. An active fragment as used herein refers to any mutant HST enzyme which is less than full length amino acid sequence to any degree. Thus, a mutant HST enzyme lacking an N-terminal peptide sequence, e.g. all or part of a transit peptide, may be considered as a core sequence, i.e. an active fragment. Various combinations of core mutant HST sequence and N-terminal region sequences, e.g. transit peptides, are possible. Therefore chimeric mutant HSTs of the invention may be designed in a mix and match approach. As already explained the motif may be modified and replaced as a further mix and match component in a design strategy.

Also by way of explanation of active fragments of mutant HST enzymes of the invention, these may include amino acid sequences of core HST enzyme sequence, or core plus transit peptide sequences, with one or more deletions of amino acids from the N-terminal and/or C-terminal ends thereof. For example, 1, 2, 3, 4, 5, 6, 7, 8, 9, 10, 11 or 12 amino acid deletions from the N-terminal and/or C-terminal ends thereof.

In any aspect of the invention described herein, the HST-inhibiting compound (also referred to as an HST inhibiting herbicide) is selected from one or more of:
a. 4-[3-chloro-6-fluoro-2-[2-(2-fluoro-4-pyridyl)ethyl]phenyl]-5-hydroxy-2,6-dimethyl-pyridazin-3-one (compound B5a);
b. 4-[3-chloro-6-fluoro-2-[2-(1-methylpyrazol-4-yl)ethyl]phenyl]-5-hydroxy-2,6-dimethyl-pyridazin-3-one (compound B5b);
c. [5-[3-chloro-6-fluoro-2-[2-[4-(trifluoromethyl)phenyl]ethyl]phenyl]-1,3-dimethyl-6-oxo-pyridazin-4-yl]2-methylpropanoate (compound B5u); and
d. 4-[3-chloro-6-fluoro-2-[2-(2-methyl-1,3-benzoxazol-6-yl)ethyl]phenyl]-5-hydroxy-2,6-dimethyl-pyridazin-3-one (compound B8a).

The invention also includes a nucleic acid molecule comprising a nucleotide sequence encoding a homogentisate solanesyltransferase (HST) enzyme or active fragment thereof as defined herein. The nucleic acid molecule may be in isolated form, that is to say it is may be synthetic or natural being substantially free of other biological components and other nucleic acids through having been subject to an isolation process involving some degree of separation or purification from other components.

The invention further includes an expression vector comprising a nucleic acid as aforementioned. Such expression vectors may further comprise one or more expression regulatory sequences. The expression regulatory sequence or sequences may comprise one or more of a transcription initiation region and a translation initiation region that are functional in a plant. The expression vector may also comprise a nucleic acid sequence encoding a transit peptide, whereby upon expression the transit peptide forms part of the mutant HST enzyme or active fragment thereof of the invention. Usually, the nucleotide sequence encoding the transit peptide when present, is coterminous and in frame with the HST amino acid sequence so that the transit peptide forms the N-terminal portion of the mutant HST protein or active fragment.

Expression regulatory sequences are preferably operably linked to the nucleic acid encoding the HST enzyme or active fragment thereof.

The invention also provides a plant, plant part or plant cell comprising a mutant HST enzyme or active fragment as hereinbefore defined. Also provided is a plant, plant part or plant cell comprising a nucleic acid as hereinbefore defined, or an expression vector as hereinbefore defined. Within the context of this specification, a “plant cell” may also be considered to include a protoplast which is simply a plant cell lacking a cell wall.

In a plant, plant part or plant cell of the invention the mutant HST enzyme or active fragment thereof may be actively expressed from a nucleic acid or an expression vector as hereinbefore defined.

The phenotype of a plant, plant part or plant cell of the invention is that the plant, plant part or any cell thereof preferably has an increased resistance to an HST-inhibiting compound as compared to a corresponding wild type or control plant, plant part or cell. Thus plants in accordance with the invention are of the herbicide resistance type due to the activity of the mutated HST enzyme or active fragment thereof.

A plant, plant part or plant cell of the invention is for example at least 10-fold more resistant to an HST-inhibiting compound/herbicide than a corresponding wild type or control plant, part or cell. As can be seen in the Examples herein, the resistance to the HST compound/herbicide may be expressed as a percentage of damage to a plant or plants as measured compared to a control untreated plant or plants, according to a suitable method: e.g. measurements at time points or after a period of time of application of the compound/herbicide of one or more of e.g. growth, biomass, necrosis, chlorosis, seed yield, photosynthetic rate.

A plant, plant part or plant cell of the invention is preferably is a crop plant, e.g. sunflower, Brassica sp., cotton, sugar, beet, soybean, peanut, alfalfa, safflower, tobacco, corn, rice, wheat, rye, barley, sorghum or millet.

A plant, part or plant cell of the invention may be transgenic in the sense that it has been produced by a process which has involved a gene transfer event of some degree; that is to say genetic material from one species has been isolated and transferred and stably incorporated into the genetic material of a recipient plant using methods of gene transfer well known to a person of skill in the art. This approach may also include synthetic nucleic acid sequences produced according to design.

Alternatively, a plant, plant part or plant cell of the invention may be non-transgenic, in the sense that the genetic material of the plant, part or cell has been modified by a process involving for example Crispr-Cas based gene editing, whereby modification of the identity of an individual nucleotide base or of bases is achieved in the genome. Again, such methods of gene editing of plant genetic material and the regeneration of whole plants from the starting point of the modified plant protoplasts, plant cells or plant tissue are well known to a person of skill in the art.

The invention includes plant reproductive material capable of producing a plant as hereinbefore defined. All kinds of plant reproductive material are included in this, whether vegetative (asexual) or sexual. For example, explants, cuttings, callus, liquid cell cultures, bulbs, corms, tubers, rhizomes or seeds; or for example, microspores, pollen or ovule. Preferably though the reproductive material is a seed.

The invention includes a plant or plant part obtained from or grown from reproductive material as noted above.

The invention also provides a container comprising plant reproductive material, preferably wherein at least 10% of the reproductive material in the container is reproductive material. Any kind of container, whether open or closed may be employed and the container may contain a medium such as growth medium (whether solid or liquid) or soil or compost.

The invention therefore provides a method of controlling undesired vegetation in the vicinity of a plant as hereinbefore defined, wherein the method comprises applying an effective amount of at least one HST-inhibiting compound/herbicide to the undesired vegetation and to said plant. The undesired vegetation lacking the mutant HST of the invention is susceptible to action by the compound/herbicide such that undesired vegetation growth activity is reduced, often to such a degree that the undesired vegetation may be caused to die. The plant of the invention has the mutant HST and so is sufficiently tolerant of the compound/herbicide that it retains sufficient growth activity to outperform or outlive the undesired vegetation. In preferred situations the plant of the invention continues to grow whilst the undesired vegetation is killed.

The invention also provides a method of enhancing growth of a plant as hereinbefore defined. The method comprises controlling undesired vegetation in the vicinity of the plant comprising applying an effective amount of at least one HST-inhibiting compound/herbicide to the undesired vegetation and to the plant. The enhancement of growth arises due to growth suppression or death of the unwanted vegetation which would otherwise take resources of water, nutrients or light away the plants of the invention.

In each of the aforementioned methods, the effective amount of said HST-inhibiting compound/herbicide does not substantially inhibit the growth of the plant comprising a mutant HST.

In any of the aforementioned methods, the HST-inhibiting compound/herbicide is selected from one or more of:
(a) 4-[3-chloro-6-fluoro-2-[2-(2-fluoro-4-pyridyl)ethyl]phenyl]-5-hydroxy-2,6-dimethyl-pyridazin-3-one (compound B5a);
(b) 4-[3-chloro-6-fluoro-2-[2-(1-methylpyrazol-4-yl)ethyl]phenyl]-5-hydroxy-2,6-dimethyl-pyridazin-3-one (compound B5b);
(c) [5-[3-chloro-6-fluoro-2-[2-[4-(trifluoromethyl)phenyl]ethyl]phenyl]-1,3-dimethyl-6-oxo-pyridazin-4-yl]2-methylpropanoate (compound B5u); and
(d) 4-[3-chloro-6-fluoro-2-[2-(2-methyl-1,3-benzoxazol-6-yl)ethyl]phenyl]-5-hydroxy-2,6-dimethyl-pyridazin-3-one (compound B8a).

The undesired vegetation preferably comprises weeds; that is to say a plant which is of the wrong species in the wrong location. A person of skill in the art will readily know of the range of weed species encountered and local agronomic publications provide encylopaedic resource, for example “The encylopaedia of arable weeds” (2018) published by Agriculture and Horticulture Development Board, Stoneleigh Park, Kenilworth, Warwickshire CV8 2TL, United Kingdom. The invention includes a method for conferring increased HST-inhibiting herbicide resistance to a plant, plant part or plant cell as compared to a corresponding control or wild-type plant, part or cell, comprising the expression in the plant, part or cell of an HST enzyme as hereinbefore defined. Included are plants, plant parts or plant cells in which there is transient expression, or induced expression dependent on a trigger provided by appropriate expression control elements, e.g. temperature, light or chemically induced expression. Also included is constitutive expression of the mutant HST of the invention in modified plants.

The invention also provides a method of producing a hybrid seed comprising crossing a first plant comprising a nucleic acid molecule as hereinbefore defined, or a first plant comprising an expression vector as hereinbefore defined, with a second plant; and obtaining seeds. The invention provides hybrid seed obtained by methods of crossing as aforementioned.

The invention also provides a method of modifying a plant, plant part or plant cell to increase resistance to an HST-inhibiting herbicide as compared to a corresponding control or wild-type plant, comprising transforming the plant, plant part, plant cell or protoplast with:
(a) a nucleic acid molecule of the invention as hereinbefore defined, or an expression vector of the invention as hereinbefore defined; and/or
(b) one or more nucleic acid molecules encoding a gene editing system for modifying an endogenous nucleic acid sequence of the plant encoding an HST enzyme at one or more positions to produce a nucleic acid sequence of the invention as hereinbefore defined.

The invention further provides a method of modifying an aforementioned plant, wherein a transformed plant part, transformed plant cell or transformed protoplast is regenerated to provide a modified plant.

Step (b) in the aforementioned method may comprise editing of the endogenous nucleic acid sequence of the plant encoding an HST enzyme. When used, a gene editing system may be a CRISPR-Cas gene editing system.

More generally, a person of skill in the art will readily appreciate the various techniques available for modifying genes in plants: see for example Wada, N., et al., (2020) “Precision genome editing in plants: state-of-the-art in CRISPR/Cas9-based genome engineering” BMC Plant Biology volume 20, Article number: 234, which is incorporated herein by reference.

In another method of modifying a plant in accordance with the invention, the obtaining of a modified plant or modified plant part may comprise selecting a plant, plant part or plant cell whose growth is partially affected or unaffected by an HST-inhibiting compound or herbicide. Such a method may involve simply subjecting a selected cohort of unmodified plant material to a mutagenic agent, whether chemical and/or physical, and then growing or regenerating and growing the mutagenized plant material, following which the plant material is subjected to challenge with selected HST-inhibiting compounds or herbicides. In this way, spontaneous mutants resistant to HST-inhibiting compounds or herbicides may be obtained.

The invention includes a modified plant, plant part or plant cell produced by any of the aforementioned methods. Such a modified plant, plant part or plant cell expresses an mutant HST enzyme or active fragment thereof as herein described. Ideally, but not necessarily a modified plant, plant part or plant cell is a genetically altered transformant.

The measure of whether a modified plant, plant part or plant cell of the invention has the necessary level of resistance to HST-inhibiting compound or herbicide is achieved by comparing growth activity with a control (i.e. unmutated or non-modified) plant. The modified plant, plant part or plant cell has an increased resistance to an HST-inhibiting herbicide as compared to the control or wild type plant, plant part or plant cell.

### BRIEF DESCRIPTION OF FIGURES

FIG. 1 shows a schematic of the process used to identify HST mutants with HST inhibitor resistance. Cells are grown in media including an HST inhibitor and subjected to UV radiation, leading to mutations in the cell's DNA. The mutations are then incorporated into the genome of cells. Cells that have mutations that provide HST inhibitor resistance survive on or in the culture medium. Thereby allowing selection of HST inhibitor resistant cells. Sequencing is then used to identify the mutations in the HST inhibitor resistant cells. The proteins encoded by the mutated genes are then identified.

FIG. 2 shows alignment of HST enzymes from various plant species listed on UniProtKB as HST enzymes with Arabidopsis Thaliana HST. Dots represent the same amino acid and shading represents similar (i.e. same charge; shape; or properties) amino acids. The region (motif) where mutations of the invention occur is highlighted by the box.

FIG. 3 shows alignment of HST enzymes from various plant species listed identified by a BLAST search with Arabidopsis Thaliana HST. This figure includes sequences which are not yet annotated or defined as being HST enzymes. Dots represent the same amino acid and shading represents similar (i.e. same charge; shape; or properties) amino acids. The region (motif) where mutations of the invention occur is highlighted by the box.

### DETAILED DESCRIPTION

### Isolated Nucleic Acids and Proteins

The invention provides isolated nucleic acid molecules that encode a functional mutant homogentisate solanesyltransferase (HST) enzyme or fragment thereof. As such, also provided are HST enzymes or functional fragments thereof that may be expressed from such isolated nucleic acids.

An “isolated” nucleic acid molecule is substantially separated away from other nucleic acid sequences with which the nucleic acid is normally associated, such as, from the chromosomal or extrachromosomal DNA of a cell in which the nucleic acid naturally occurs. A nucleic acid molecule may be an isolated nucleic acid molecule when it comprises a transgene or part of a transgene present in the genome of another organism. The term also embraces nucleic acids that are biochemically purified so as to substantially remove contaminating nucleic acids and other cellular components. Isolated nucleic acids are substantially free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. The isolated nucleic acid molecule may be flanked by its native genomic sequences that control its expression in the cell, for example, the native promoter, or native 3′ untranslated region.

A protein or enzyme that is substantially free of cellular material includes preparations of protein or enzyme having less than about 30%, 20%, 10%, 5%, or 1% (by dry weight) of contaminating protein. When the protein or enzyme of the invention or functional fragment thereof is recombinantly produced, preferably culture medium represents less than about 30%, 20%, 10%, 5%, or 1% (by dry weight) of chemical precursors or non-protein-of-interest chemicals.

As used herein, reference to an “HST enzyme” and the amino acid sequences thereof also refers to and is intended to encompass isolated nucleic acids encoding such HST enzyme.

Homogentisate solanesyl transferase (HST) refers to an enzyme that catalyzes the prenylation and decarboxylation of homogentisate to form 2-methyl-6-solanesyl-1,4-benzoquinol, the first intermediate in plastoquinone-9 biosynthesis. HST may be located in the inner envelope membrane of chloroplasts. HST is identified by the Enzyme Commission (EC) number 2.5.1.117. Homogentisate solanesyl transferase (HST) enzyme may refer to any polypeptide capable of catalysing the prenylation and decarboxylation of homogentisate to form 2-methyl-6-solanesyl-1,4-benzoquinol, the first intermediate in plastoquinone-9 biosynthesis. Examples of HST include those identified by the UNIProtKB numbers Q1ACB3 (Arabidopsis thaliana HST), F4J8K0 (Arabidopsis thaliana HST), and A1JHN0 (Chlamydomonas reinhardtii HST). HST may also be referred to as homogentisate prenyltransferase.

The HST enzyme may have an amino acid sequence which includes the motif sequence:

(SEQ ID NO: 1)
F[V/M]TX[F/Y]

“X” may be any amino acid. In the motif, square brackets denote interchangeable amino acids. For example, [V/M] denotes a residue that is either valine or methionine. In some examples X may be selected from a neutral amino acid or an hydrophobic amino acid; preferably X is selected from one of leucine, methionine, phenylalanine, isoleucine, valine, threonine or cysteine.

The HST enzymes of the invention are mutated enzymes and may include at least one mutation of at least on residue of the motif of SEQ ID NO: 1. By mutation is meant any substitution, deletion or insertion of 1, 2, 3, 4 or 5 amino acids. Mutated enzymes of the invention may comprise such a mutation at any one or more of positions 1, 2, 3, 4 of 5 of SEQ ID NO: 1.

In particular the first amino acid (position 1) of the motif may be substituted. For example, by a non-conservative substitution. In some examples, residue 1 is substituted with an aliphatic amino acid. For example, the HST enzyme may comprise the motif:

(SEQ ID NO: 2)
I[V/M]TX[F/Y]

The HST enzymes of the invention are mutated enzymes and may include at least mutated residue of the motif of SEQ ID NO: 1. In particular residue 2 (i.e. position 2) of the motif may be substituted. For example, by a conservative substitution. In some examples, residue 2 is substituted with an aliphatic amino acid. For example, the HST enzyme may comprise the motif:

(SEQ ID NO: 3)
FATX[F/Y]

The HST enzymes of the invention are mutated enzymes and may include at least mutated residue of the motif of SEQ ID NO: 1. In particular residue 3 (i.e. position 3) of the motif may be substituted. For example, by a non-conservative substitution. In some examples, residue 3 is substituted with an acidic amino acid. For example, the HST enzyme may comprise the motif:

(SEQ ID NO: 4)
F[V/M]IX[F/Y]

The HST enzymes of the invention are mutated enzymes and may include at least mutated residue of the motif of SEQ ID NO: 1. In particular residue 5 (i.e. position 5) of the motif may be substituted. For example, by a non-conservative substitution. In some examples, residue 2 is substituted with an aliphatic amino acid. For example, the HST enzyme may comprise the motif:

(SEQ ID NO: 5)
F[V/M]TXI
or
(SEQ ID NO: 6)
F[V/M]IXL

Alternatively, the mutant HST enzyme may comprise the motif:

(SEQ ID NO: 7)
[T/A]X₂F[V/M]TX[F/Y]A X₃X₄IX₅X₆X₇KDLX₈D

Wherein “X” may be any amino acid. In the motif square brackets denote interchangeable amino acids. In some examples X may be selected from one of leucine, methionine, phenylalanine, isoleucine, valine, threonine or cysteine. X₂ to X₈ may be any amino acid. In some examples X₂ may be selected from serine, cysteine, threonine, alanine, arginine, glycine or valine. In some examples, X₃ may be selected from leucine, serine, cysteine, threonine, alanine or valine. In some examples, X₄ may be selected from valine, alanine or isoleucine. In some examples, X₅ may be selected from alanine or serine. In some examples, X₆ may be selected from isoleucine, valine, leucine or alanine. In some examples, X₇ may be selected from threonine, serine or alanine. In some examples, X₈ may be selected from proline, alanine or glycine. Any combination of any number of the variable positions of the motif may be changed according to design or requirement.

As such, a mutated HST enzyme of the invention may comprise a motif according to any of SEQ ID NOs 8 to 12:

(SEQ ID NO: 8)
[T/A]X₂I[V/M]TX[F/Y]A X₃X₄IX₅X₆X₇KDLX₈D
(SEQ ID NO: 9)
[T/A]X₂FATX[F/Y]A X₃X₄IX₅X₆X₇KDLX₈D
(SEQ ID NO: 10)
[T/A]X₂F[V/M]IX[F/Y]A X₃X₄IX₅X₆X₇KDLX₈D
(SEQ ID NO: 11)
[T/A]X₂F[V/M]TXIA X₃X₄IX₅X₆X₇KDLX₈D
(SEQ ID NO: 12)
[T/A]X₂F[V/M]TXLA X₃X₄IX₅X₆X₇KDLX₈D.

An HST enzyme of the invention may comprise an amino acid sequence according to SEQ ID NO: 13 wherein positions 196 to 200 thereof include at least one mutation as described herein:

(SEQ ID NO: 13)
VGAAESDDPVLDRIARFQNACWRFLRPHTIRGTALGSTALVTRAL
IENTHLIKWSLVLKALSGLLALICGNGYIVGINQIYDIGIDKVNK
PYLPIAAGDLSVQSAWLLVIFFAIAGLLVVGFNFGPFITSLYSLG
LFLGTIYSVPPLRMKRFPVAAFLIIATVRGFLLNFGVYHATRAAL
GLPFQWSAPVAFITSFVTLFALVIAITKDLPDVEGDRKFQISTLA
TKLGVRNIAFLGSGLLLVNYVSAISLAFYMPQVFRGSLMIPAHVI
LASGLIFQTWVLEKANYTKEAISGYYRFIWNLFYAEYLLFPFL

The HST enzyme may comprise an amino acid sequence that has at least 70% sequence identity to an amino acid sequence according to SEQ ID NO: 13, wherein positions 196 to 200 thereof include at least one mutation as described herein. Other variants of such an HST enzyme may comprise an amino acid sequence of at least 75%, at least 80%, at least 85%, at least 90%, at least 95%, at least 98% or at least 99% sequence identity to an amino acid sequence according to SEQ ID NO: 13, wherein positions 196 to 200 thereof include at least one mutation as described herein.

Positions 196 to 200 of SEQ ID NO: 13 correspond to positions 276 to 280 of SEQ ID NO: 14 (which relates to Arabidopsis thaliana HST). As such, the HST enzyme may have an amino acid sequence according to SEQ ID NO: 14 wherein positions 276 to 280 thereof include at least one mutation as described herein:

(SEQ ID NO: 14)
MELSISQSPRVRFSSLAPRFLAASHHHRPSVHLAGKFISLPRDVR
FTSLSTSRMRILAVALTFKSRCVYVNYEIPKDQILVGAAESDDPV
LDRIARFQNACWRFLRPHTIRGTALGSTALVTRALIENTHLIKWS
LVLKALSGLLALICGNGYIVGINQIYDIGIDKVNKPYLPIAAGDL
SVQSAWLLVIFFAIAGLLVVGFNFGPFITSLYSLGLFLGTIYSVP
PLRMKRFPVAAFLIIATVRGFLLNFGVYHATRAALGLPFQWSAPV
AFITSFVTLFALVIAITKDLPDVEGDRKFQISTLATKLGVRNIAF
LGSGLLLVNYVSAISLAFYMPQVFRGSLMIPAHVILASGLIFQTW
VLEKANYTKEAISGYYRFIWNLFYAEYLLFPFL

The HST enzyme may comprise an amino acid sequence that has at least 70% sequence identity to an amino acid sequence according to SEQ ID NO: 1. Other variants of such an HST enzyme may comprise an amino acid sequence of at least 75%, at least 80%, at least 85%, at least 90%, at least 95%, at least 98% or at least 99% sequence identity to an amino acid sequence according to SEQ ID NO: 14 wherein positions 276 to 280 thereof include at least one mutation as described herein.

Positions 196 to 200 of SEQ ID NO: 13 correspond to positions 200 to 204 of SEQ ID NO: 15 (which relates to Arabidopsis thaliana HST, not including a transit peptide). As such, the HST enzyme may have an amino acid sequence according to SEQ ID NO: 15 wherein positions 200 to 204 thereof include at least one mutation as described herein:

(SEQ ID NO: 15)
ACSQVGAAESDDPVLDRIARFQNACWRFLRPHTIRGTALGSTALV
TRALIENTHLIKWSLVLKALSGLLALICGNGYIVGINQIYDIGID
KVNKPYLPIAAGDLSVQSAWLLVIFFAIAGLLVVGFNFGPFITSL
YSLGLFLGTIYSVPPLRMKRFPVAAFLIIATVRGFLLNFGVYHAT
RAALGLPFQWSAPVAFITSFVTLFALVIAITKDLPDVEGDRKFQI
STLATKLGVRNIAFLGSGLLLVNYVSAISLAFYMPQVFRGSLMIP
AHVILASGLIFQTWVLEKANYTKEAISGYYRFIWNLFYAEYLLFP
F

The HST enzyme may comprise an amino acid sequence that has at least 70% sequence identity to an amino acid sequence according to SEQ ID NO: 15 wherein positions 200 to 204 thereof include at least one mutation as described herein. Other variants of such an HST enzyme may comprise an amino acid sequence of at least 75%, at least 80%, at least 85%, at least 90%, at least 95%, at least 98% or at least 99% sequence identity to an amino acid sequence according to SEQ ID NO: 15 wherein positions 200 to 204 thereof include at least one mutation as described herein. The nucleic acid sequence may encode an HST enzyme having a sequence according SEQ ID NO:15.

Positions 196 to 200 of SEQ ID NO: 13 correspond to positions 269 to 273 of SEQ ID NO: 16 (which relates to Arabidopsis thaliana HST, including the naturally occurring transit peptide. As such, the HST enzyme may have an amino acid sequence according to SEQ ID NO: 16 wherein positions 269 to 273 thereof include at least one mutation as described herein:

(SEQ ID NO: 16)
MELSISQSPRVRFSSLAPRFLAASHHHRPSVHLAGKFISLPRDVR
FTSLSTSRMRSKFVSTNYRKISIRACSQVGAAESDDPVLDRIARF
QNACWRFLRPHTIRGTALGSTALVTRALIENTHLIKWSLVLKALS
GLLALICGNGYIVGINQIYDIGIDKVNKPYLPIAAGDLSVQSAWL
LVIFFAIAGLLVVGFNFGPFITSLYSLGLFLGTIYSVPPLRMKRF
PVAAFLIIATVRGFLLNFGVYHATRAALGLPFQWSAPVAFITSFV
TLFALVIAITKDLPDVEGDRKFQISTLATKLGVRNIAFLGSGLLL
VNYVSAISLAFYMPQVFRGSLMIPAHVILASGLIFQTWVLEKANY
TKEAISGYYRFIWNLFYAEYLLFPFL

The HST enzyme may comprise an amino acid sequence that has at least 70% sequence identity to an amino acid sequence according to SEQ ID NO: 16, wherein positions 269 to 273 thereof include at least one mutation as described herein. Other variants of such an HST enzyme may comprise an amino acid sequence of at least 75%, at least 80%, at least 85%, at least 90%, at least 95%, at least 98% or at least 99% sequence identity to an amino acid sequence according to SEQ ID NO: 16 wherein positions 269 to 273 include at least one mutation as described herein. The nucleic acid sequence may encode an HST enzyme having a sequence according SEQ ID NO:16, wherein positions 269 to 273 include at least one mutation as described herein.

The HST enzyme of the invention may comprise an amino acid sequence according to SEQ ID NO: 17 (which relates to Oryza sativa HST, not including a transit peptide), wherein positions 199 to 203 thereof include at least one mutation as described herein:

(SEQ ID NO: 17)
VCSQAGAAGPAPLSKTLSDLKDSCWRFLRPHTIRGTALGSIALVA
RALIENPQLINWWLVFKAFYGLVALICGNGYIVGINQIYDIRIDK
VNKPYLPIAAGDLSVQTAWLLVVLFAAAGFSIVVTNFGPFITSLY
CLGLFLGTIYSVPPFRLKRYPVAAFLIIATVRGFLLNFGVYYATR
AALGLTFQWSSPVAFITCFVTLFALVIAITKDLPDVEGDRKYQIS
TLATKLGVRNIAFLGSGLLIANYVAAIAVAFLMPQAFRRTVMVPV
HAALAVGIIFQTWVLEQAKYTKDAISQYYRFIWNLFYAEYIFFPL
I

The HST enzyme may comprise an amino acid sequence that has at least 70% sequence identity to an amino acid sequence according to SEQ ID NO: 17, wherein positions 199 to 203 thereof include at least one mutation as described herein. Other variants of such an HST enzyme may comprise an amino acid sequence of at least 75%, at least 80%, at least 85%, at least 90%, at least 95%, at least 98% or at least 99% sequence identity to an amino acid sequence according to SEQ ID NO: 17, wherein positions 199 to 203 include at least one mutation as described herein.

The HST enzyme of the invention may comprise an amino acid sequence according to SEQ ID NO: 18 (which relates to Oryza sativa HST, including the naturally occurring chloroplast transit peptide), wherein positions 261 to 265 thereof include at least one mutation as described herein:

(SEQ ID NO: 18)
MASLASPPLPCRAAATASRSGRPAPRLLGPPPPPASPLLSSASAR
FPRAPCNAARWSRRDAVRVCSQAGAAGPAPLSKTLSDLKDSCWRF
LRPHTIRGTALGSIALVARALIENPQLINWWLVFKAFYGLVALIC
GNGYIVGINQIYDIRIDKVNKPYLPIAAGDLSVQTAWLLVVLFAA
AGFSIWVTNFGPFITSLYCLGLFLGTIYSVPPFRLKRYPVAAFLI
IATVRGFLLNFGVYYATRAALGLTFQWSSPVAFITCFVTLFALVI
AITKDLPDVEGDRKYQISTLATKLGVRNIAFLGSGLLIANYVAAI
AVAFLMPQAFRRTVMVPVHAALAVGIIFQTWVLEQAKYTKDAISQ
YYRFIWNLFYAEYIFFPLI

The HST enzyme may comprise an amino acid sequence that has at least 70% sequence identity to an amino acid sequence according to SEQ ID NO: 18, wherein positions 261 to 265 include at least one mutation as described herein. Other variants of such an HST enzyme may comprise an amino acid sequence of at least 75%, at least 80%, least 85%, at least 90%, at least 95%, at least 98% or at least 99% sequence identity to an amino acid sequence according to SEQ ID NO: 18, wherein positions 261 to 265 include at least one mutation as described herein.

The HST enzyme of the invention may comprise an amino acid sequence according to SEQ ID NO: 19 (which relates to Chlorella fusca HST and which does not include a chloroplast transit peptide) wherein positions 198 to 202 thereof include at least one mutation as described herein:

(SEQ ID NO: 19)
SSTYSGPDGWSGDGSQGSFLSAFWRFLRPHTIRGTILGTSAVVTK
VVLSNMEAIDWGLLPRALMGLVALLCGNGYIVGINQIYDVDIDAV
NKPFLPVAAGDMSPGTAWVLVLALAAAGLGIVATNFGNLITGLYG
FGLLLGTVYSVPPLRLKRFAVPAFLIIATVRGFLLNFGVYHATRA
ALQLPFEWSPAILFITCFVTMFATVIAITKDLPDIEGDKANNIST
FATRLGVKNVSLLGVGMLLLNYVMAGVFALKYSSYFNVPVMLGAH
ALLALVLILRTTKLAAAGYTQQAVISFYRWIWNLFYSEYLVYVLI

The HST enzyme may comprise an amino acid sequence that has at least 70% sequence identity to an amino acid sequence according to SEQ ID NO: 19, wherein positions 198 to 202 thereof include at least one mutation as described herein. Other variants of such an HST enzyme may comprise an amino acid sequence of at least 75%, at least 80%, at least 85%, at least 90%, at least 95%, at least 98% or at least 99% sequence identity to an amino acid sequence according to SEQ ID NO: 19, wherein positions 198 to 202 include at least one mutation as described herein.

The HST enzyme of the invention may comprise an amino acid sequence according to SEQ ID NO: 20 (which relates to a Chlorella fusca HST), wherein positions 275 to 279 thereof include at least one mutation as described herein:

(SEQ ID NO: 20)
MLSSGQNQVQQSLGRHKSSRWSCSHAFQPSRTSQWPRLLPGLHSK
VQQQHHLHQLPQQLQQHQLQQPERLVSTQAASSSTYSGPDGWSGD
GSQGSFLSAFWRFLRPHTIRGTILGTSAVVTKVVLSNMEAIDWGL
LPRALMGLVALLCGNGYIVGINQIYDVDIDAVNKPFLPVAAGDMS
PGTAWVLVLALAAAGLGIVATNFGNLITGLYGFGLLLGTVYSVPP
LRLKRFAVPAFLIIATVRGFLLNFGVYHATRAALQLPFEWSPAIL
FITCFVTMFATVIAITKDLPDIEGDKANNISTFATRLGVKNVSLL
GVGMLLLNYVMAGVFALKYSSYFNVPVMLGAHALLALVLILRTTK
LAAAGYTQQAVISFYRWIWNLFYSEYLVYVLI

The HST enzyme may comprise an amino acid sequence that has at least 70% sequence identity to an amino acid sequence according to SEQ ID NO: 20 wherein positions 275 to 279 include at least one mutation as described herein. Other variants of such an HST enzyme may comprise an amino acid sequence of at least 75%, at least 80%, at least 85%, at least 90%, at least 95%, at least 98% or at least 99% sequence identity to an amino acid sequence according to SEQ ID NO: 20, wherein positions 275 to 279 include at least one mutation as described herein.

The HST enzyme of the invention may comprise an amino acid sequence according to SEQ ID NO: 21 (which relates to Chlorella fusca HST including the naturally occurring chloroplast transit peptide) wherein positions 261 to 265 thereof include at least one mutation as described herein:

(SEQ ID NO: 21)
MLRSSRWSCSHAFQPSRTSQWPRLLPGLHSKVQQQHHLHQLPQQL
QQHQLQQPERLVSTQAASSSTYSGPDGWSGDGSQGSFLSAFWRFL
RPHTIRGTILGTSAVVTKVVLSNMEAIDWGLLPRALMGLVALLCG
NGYIVGINQIYDVDIDAVNKPFLPVAAGDMSPGTAWVLVLALAAA
GLGIVATNFGNLITGLYGFGLLLGTVYSVPPLRLKRFAVPAFLII
ATVRGFLLNFGVYHATRAALQLPFEWSPAILFITCFVTMFATVIA
ITKDLPDIEGDKANNISTFATRLGVKNVSLLGVGMLLLNYVMAGV
FALKYSSYFNVPVMLGAHALLALVLILRTTKLAAAGYTQQAVISF
YRWIWNLFYSEYLVYVLI

The HST enzyme may comprise an amino acid sequence that has at least 70% sequence identity to an amino acid sequence according to SEQ ID NO: 21, wherein positions 261 to 265 include at least one mutation as described herein. Other variants of such an HST enzyme may comprise an amino acid sequence of at least 75%, at least 80%, at least 85%, at least 90%, at least 95%, at least 98% or at least 99% sequence identity to an amino acid sequence according to SEQ ID NO: 21, wherein positions 261 to 265 include at least one mutation as described herein.

In any aspect of the invention as described herein, an HST enzyme which has a degree of HST-inhibitor resistance, whether partial or complete, may comprise an amino acid which has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any of SEQ ID NOs 13-21 herein, which may also be referred to as reference sequences.

In any aspect of the invention, an HST enzyme which has HST-inhibitor resistance may be a homologue of any of SEQ ID NOs 13 to 21. As used herein, “homologue” refers to a protein that is functionally equivalent i.e. has the same enzymatic activity as an enzyme having an amino acid sequence according to SEQ ID NO 13 to 21 (i.e. acts as an HST enzyme as defined herein), but may have a limited number of amino acid substitutions, deletions, insertions or additions in the amino acid sequence. Homologues may have lower sequences identities, for example at least 20%, at least 25%, at least 30%, at least 35% or at least 40% or more sequence identity to an HST enzyme identified herein, but are capable of carrying out the same enzymatic reaction (i.e. that identified by Enzyme Commission (EC) number 2.5.1.117). The invention therefore includes any isoforms of HST enzymes and their mutations as defined herein.

“Identity” or “percent identity” refers to the degree of sequence variation between two given nucleic acid or amino acid sequences. For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math.2: 482 (1981), by the homology alignment algorithm of Needleman & Wunsch, J. Mol. Biol.48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection. One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., J. Mol. Biol. 215: 403-410 (1990)). These initial neighbourhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see, Henikoff & Henikoff, Proc. Natl. Acad. Sci. USA 89: 10915 (1989)). In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

An HST enzyme encoded by a nucleic acid or an HST enzyme of the invention may be a functional fragment of an HST enzyme as described herein A to a protein fragment that retains protein function. As such, a functional fragment of an HST enzyme is a fragment, portion or part of an HST protein that is capable of catalysing the prenylation and decarboxylation of homogentisate to form 2-methyl-6-solanesyl-1,4-benzoquinol.

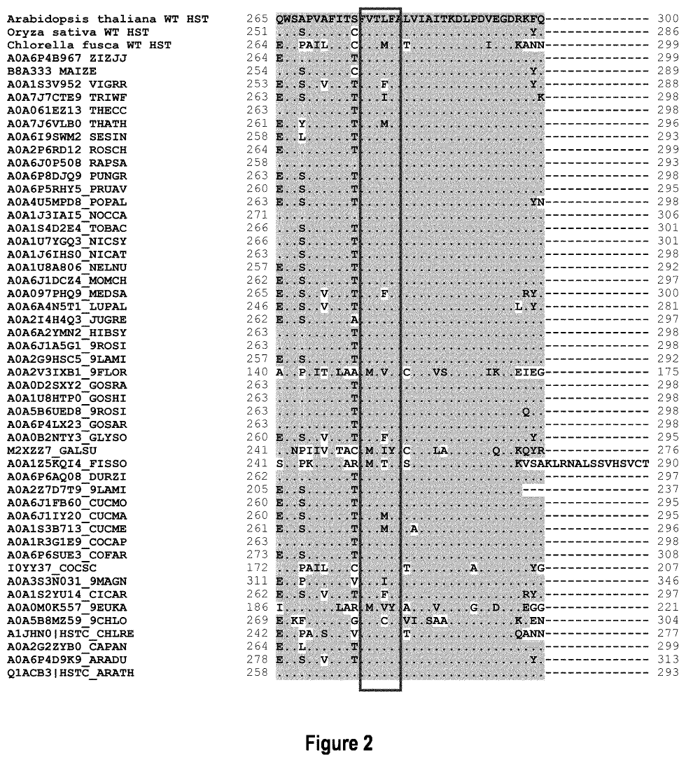

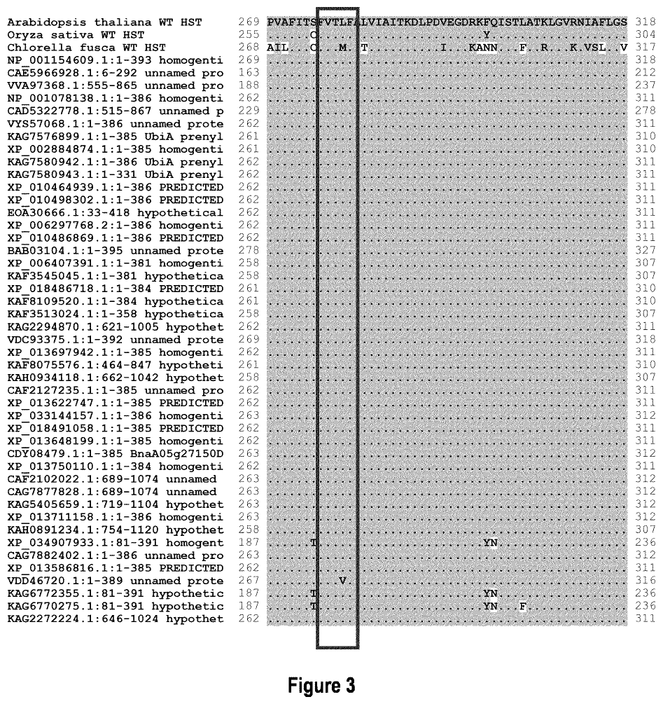

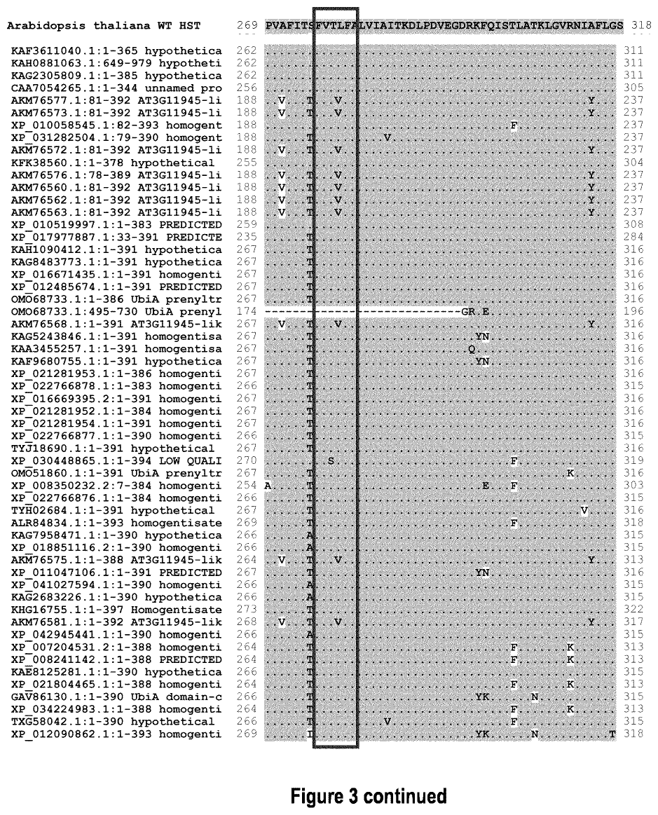

The mutation may be located at a position corresponding to an amino acid position denoted above in other HST enzymes. It is possible to compare HST polypeptides by sequence comparison and locating conserved regions that correspond to the amino acid positions denoted above as is shown in FIG. 2 which provides an alignment of HST enzymes from various origins. The term “equivalent amino acids” or “corresponding amino acids” refers to amino acids in a first sequence which correspond to those of an identified reference strain. A region of equivalent amino acids may be determined by aligning the amino acid sequences of the proteins from the different species, using an alignment program such as BLAST® or ClustalW.

Mutations may include deletions or substitutions or combinations thereof. For example, the mutations may be conservative or non-conservative amino acid substitutions.

“Conservative amino acid substitutions” refer to the interchangeability of residues having similar side chains, and thus typically involves substitution of an amino acid in a polypeptide with amino acids within the same or similar defined class of amino acids. By way of example, an amino acid with an aliphatic side chain may be substituted with another aliphatic amino acid, e.g., alanine, valine, leucine, and isoleucine; an amino acid with hydroxyl side chain may be substituted with another amino acid with a hydroxyl side chain, e.g., serine and threonine; an amino acids having aromatic side chains may be substituted with another amino acid having an aromatic side chain, e.g., phenylalanine, tyrosine, tryptophan, and histidine; an amino acid with a basic side chain may be substituted with another amino acid with a basic side chain, e.g., lysine and arginine; an amino acid with an acidic side chain may be substituted with another amino acid with an acidic side chain, e.g., aspartic acid or glutamic acid; and a hydrophobic or hydrophilic amino acid may be substituted with another hydrophobic or hydrophilic amino acid, respectively. Exemplary conservative substitutions are provided below:

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C | None |
| P | None |

“Non-conservative substitution” refers to substitution of an amino acid in a polypeptide with an amino acid with significantly differing side chain properties. Non-conservative substitutions may use amino acids between, rather than within, the defined groups and may affect (a) the structure of the peptide backbone in the area of the substitution (e.g., proline for glycine) (b) the charge or hydrophobicity, or (c) the bulk of the side chain. By way of example, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification of a polypeptide by removal of one or more amino acids in comparison to a wild-type or control polypeptide. Deletions can comprise removal of 1 or more amino acids, 2 or more amino acids, or 3 or more amino acids of the polypeptide while retaining enzymatic activity. Deletions can comprise a continuous segment or can be discontinuous.

The mutation may be located a position selected from amino acid positions 196, 197, 198, 199 and 200 of SEQ ID NO: 13. For example, the HST enzyme may include at least one mutation of amino acids at positions 196, 197, 198, 199 and/or 200 of SEQ ID NO: 13.

The HST enzyme includes at least one mutation. The mutation may be located a position selected from amino acid positions 276, 277, 278, 279 and 280 of SEQ ID NO: 14. For example, the HST enzyme may include at least one mutation of amino acids at positions 276, 277, 278, 279 and/or 280 of SEQ ID NO: 14.

The mutation may be located a position selected from amino acid positions 200, 201, 202, 203, and 204 of SEQ ID NO: 15. For example, the HST enzyme may include at least one mutation of amino acids at positions 200, 201, 202, 203, and/or 204 of SEQ ID NO: 15.

The mutation may be located a position selected from amino acid positions 269, 270, 271, 272 and 273 of SEQ ID NO: 16. For example, the HST enzyme may include at least one mutation of amino acids at positions 269, 270, 271, 272 and/or 273 of SEQ ID NO: 16.

The mutation may be located a position selected from amino acid positions 199, 200, 201, 202, 203 and 204 of SEQ ID NO: 17. For example, the HST enzyme may include at least one mutation of amino acids at positions 199, 200, 201, 202, 203 and/or 204 of SEQ ID NO: 17.

The mutation may be located a position selected from amino acid positions 262, 263, 264, 265 and/or 266 of SEQ ID NO 18. For example, the HST enzyme may include at least one mutation of amino acids at positions 262, 263, 264, 265 and/or 266 of SEQ ID NO: 18.

The mutation may be located a position selected from amino acid positions 198, 199, 200, 201 and 202 of SEQ ID NO: 19. For example, the HST enzyme may include at least one mutation of amino acids at positions 198, 199, 200, 201 and/or 202 of SEQ ID NO: 19.

The mutation may be located a position selected from amino acid positions 275, 276, 277, 278, and 279 of SEQ ID NO: 20. For example, the HST enzyme may include at least one mutation of amino acids at positions 276, 277, 278 and/or 279 of SEQ ID NO: 20.

The mutation may be located a position selected from amino acid positions 261, 262, 263, 246 and 275 of SEQ ID NO: 21. For example, the HST enzyme may include at least one mutation of amino acids at positions 261, 262, 263, 246 and/or 275 of SEQ ID NO: 21.

For example, the HST enzyme may include an amino acid sequence that has a mutation at position 196 of SEQ ID NO:13. For example, the mutation may be a substitution of F196. The substitution may be a non-conservative mutation. F196 may be substituted with an aliphatic amino acid residue. For example, the mutation may be F196I. For example, there is provided herein a nucleic acid molecule encoding an HST enzyme and an HST enzyme that include an amino acid sequence of Arabidopsis thaliana F196I:

(SEQ ID NO: 22)
VGAAESDDPVLDRIARFQNACWRFLRPHTIRGTALGSTALVTRAL
IENTHLIKWSLVLKALSGLLALICGNGYIVGINQIYDIGIDKVNK
PYLPIAAGDLSVQSAWLLVIFFAIAGLLVVGFNFGPFITSLYSLG
LFLGTIYSVPPLRMKRFPVAAFLIIATVRGFLLNFGVYHATRAAL
GLPFQWSAPVAFITSIVTLFALVIAITKDLPDVEGDRKFQISTLA
TKLGVRNIAFLGSGLLLVNYVSAISLAFYMPQVFRGSLMIPAHVI
LASGLIFQTWVLEKANYTKEAISGYYRFIWNLFYAEYLLFPFL

For example, the HST enzyme may include an amino acid sequence that has a mutation at position 197 of SEQ ID NO:13. For example, the mutation may be a substitution of V197. The substitution may be a conservative mutation. V197 may be substituted with an aliphatic amino acid residue. For example, the mutation may be V197A. For example, there is provided herein a nucleic acid molecule encoding an HST enzyme and an HST enzyme that include an amino acid sequence of Arabidopsis thaliana V197A:

(SEQ ID NO: 23)
VGAAESDDPVLDRIARFQNACWRFLRPHTIRGTALGSTALVTRAL
IENTHLIKWSLVLKALSGLLALICGNGYIVGINQIYDIGIDKVNK
PYLPIAAGDLSVQSAWLLVIFFAIAGLLVVGFNFGPFITSLYSLG
LFLGTIYSVPPLRMKRFPVAAFLIIATVRGFLLNFGVYHATRAAL
GLPFQWSAPVAFITSFATLFALVIAITKDLPDVEGDRKFQISTLA
TKLGVRNIAFLGSGLLLVNYVSAISLAFYMPQVFRGSLMIPAHVI
LASGLIFQTWVLEKANYTKEAISGYYRFIWNLFYAEYLLFPFL

For example, the HST enzyme may include an amino acid sequence that has a mutation at position 198 of SEQ ID NO:13. For example, the mutation may be a substitution of T198. The substitution may be a non-conservative mutation. T198 may be substituted with an acidic amino acid residue. For example, the mutation may be T198N. For example, there is provided herein a nucleic acid molecule encoding an HST enzyme and an HST enzyme that include an amino acid sequence of Arabidopsis thaliana T198N:

(SEQ ID NO: 24)
VGAAESDDPVLDRIARFQNACWRFLRPHTIRGTALGSTALVTRAL
IENTHLIKWSLVLKALSGLLALICGNGYIVGINQIYDIGIDKVNK
PYLPIAAGDLSVQSAWLLVIFFAIAGLLVVGFNFGPFITSLYSLG
LFLGTIYSVPPLRMKRFPVAAFLIIATVRGFLLNFGVYHATRAAL
GLPFQWSAPVAFITSFVNLFALVIAITKDLPDVEGDRKFQISTLA
TKLGVRNIAFLGSGLLLVNYVSAISLAFYMPQVFRGSLMIPAHVI
LASGLIFQTWVLEKANYTKEAISGYYRFIWNLFYAEYLLFPFL

The HST enzyme may have a mutation at position 200 of SEQ ID NO:13. The mutation may be a substitution of F200. The substitution may be a non-conservative mutation. F200 may be substituted with an aliphatic amino acid residue. For example, the mutation may be F200I. For example, the mutation may be F200L. For example, there is provided herein a nucleic acid molecule encoding an HST enzyme and an HST enzyme that include an amino acid sequence of Arabidopsis thaliana F200I:
according to:

(SEQ ID NO: 25)
VGAAESDDPVLDRIARFQNACWRFLRPHTIRGTALGSTALVTRAL
IENTHLIKWSLVLKALSGLLALICGNGYIVGINQIYDIGIDKVNK
PYLPIAAGDLSVQSAWLLVIFFAIAGLLVVGFNFGPFITSLYSLG
LFLGTIYSVPPLRMKRFPVAAFLIIATVRGFLLNFGVYHATRAAL
GLPFQWSAPVAFITSFVTLIALVIAITKDLPDVEGDRKFQISTLA
TKLGVRNIAFLGSGLLLVNYVSAISLAFYMPQVFRGSLMIPAHVI
LASGLIFQTWVLEKANYTKEAISGYYRFIWNLFYAEYLLFPFL

For example, there is provided herein a nucleic acid molecule encoding an HST enzyme and an HST enzyme that include an amino acid sequence according to Arabidopsis thaliana F200L:

(SEQ ID NO: 26)
VGAAESDDPVLDRIARFQNACWRFLRPHTIRGTALGSTALVTRAL
IENTHLIKWSLVLKALSGLLALICGNGYIVGINQIYDIGIDKVNK
PYLPIAAGDLSVQSAWLLVIFFAIAGLLVVGFNFGPFITSLYSLG
LFLGTIYSVPPLRMKRFPVAAFLIIATVRGFLLNFGVYHATRAAL
GLPFQWSAPVAFITSFVTLLALVIAITKDLPDVEGDRKFQISTLA
TKLGVRNIAFLGSGLLLVNYVSAISLAFYMPQVFRGSLMIPAHVI
LASGLIFQTWVLEKANYTKEAISGYYRFIWNLFYAEYLLFPFL

For example, the HST enzyme may have a mutation at position 276 of SEQ ID NO:14. For example, the mutation may be a substitution of F276. The substitution may be a non-conservative mutation. F276 may be substituted with an aliphatic amino acid residue. For example, the mutation may be F276I. For example, there is provided herein a nucleic acid molecule encoding an HST enzyme and an HST enzyme that include an amino acid sequence according to Arabidopsis HST F276I:

(SEQ ID NO: 27)
MELSISQSPRVRFSSLAPRFLAASHHHRPSVHLAGKFISLPRDVR
FTSLSTSRMRILAVALTFKSRCVYVNYEIPKDQILVGAAESDDPV
LDRIARFQNACWRFLRPHTIRGTALGSTALVTRALIENTHLIKWS
LVLKALSGLLALICGNGYIVGINQIYDIGIDKVNKPYLPIAAGDL
SVQSAWLLVIFFAIAGLLVVGFNFGPFITSLYSLGLFLGTIYSVP
PLRMKRFPVAAFLIIATVRGFLLNFGVYHATRAALGLPFQWSAPV
AFITSIVTLFALVIAITKDLPDVEGDRKFQISTLATKLGVRNIAF
LGSGLLLVNYVSAISLAFYMPQVFRGSLMIPAHVILASGLIFQTW
VLEKANYTKEAISGYYRFIWNLFYAEYLLFPFL

The HST enzyme may have a mutation at position 277 of SEQ ID NO:14. The mutation may be a substitution of V277. The substitution may be a conservative mutation. V277 may be substituted with an aliphatic amino acid residue. For example, the mutation may be V277A. For example, there is provided herein a nucleic acid molecule encoding an HST enzyme and an HST enzyme that include an amino acid sequence according to Arabidopsis HST V277A:

(SEQ ID NO: 28)
MELSISQSPRVRFSSLAPRFLAASHHHRPSVHLAGKFISLPRDVRFTS
LSTSRMRILAVALTFKSRCVYVNYEIPKDQILVGAAESDDPVLDRIAR
FQNACWRFLRPHTIRGTALGSTALVTRALIENTHLIKWSLVLKALSGL
LALICGNGYIVGINQIYDIGIDKVNKPYLPIAAGDLSVQSAWLLVIFF
AIAGLLVVGFNFGPFITSLYSLGLFLGTIYSVPPLRMKRFPVAAFLII
ATVRGFLLNFGVYHATRAALGLPFQWSAPVAFITSFATLFALVIAITK
DLPDVEGDRKFQISTLATKLGVRNIAFLGSGLLLVNYVSAISLAFYMP
QVFRGSLMIPAHVILASGLIFQTWVLEKANYTKEAISGYYRFIWNLFY
AEYLLFPFL

The HST enzyme may have a mutation at position 278 of SEQ ID NO:14. The mutation may be a substitution of T278. The substitution may be a non-conservative mutation. T278 may be substituted with an acidic amino acid residue. For example, the mutation may be T278N. For example, there is provided herein a nucleic acid molecule encoding an HST enzyme and an HST enzyme that include an amino acid sequence according to Arabidopsis HSTT278N:

(SEQ ID NO: 29)
MELSISQSPRVRFSSLAPRFLAASHHHRPSVHLAGKFISLPRDVRFTS
LSTSRMRILAVALTFKSRCVYVNYEIPKDQILVGAAESDDPVLDRIAR
FQNACWRFLRPHTIRGTALGSTALVTRALIENTHLIKWSLVLKALSGL
LALICGNGYIVGINQIYDIGIDKVNKPYLPIAAGDLSVQSAWLLVIFF
AIAGLLVVGFNFGPFITSLYSLGLFLGTIYSVPPLRMKRFPVAAFLII
ATVRGFLLNFGVYHATRAALGLPFQWSAPVAFITSFVNLFALVIAITK
DLPDVEGDRKFQISTLATKLGVRNIAFLGSGLLLVNYVSAISLAFYMP
QVFRGSLMIPAHVILASGLIFQTWVLEKANYTKEAISGYYRFIWNLFY
AEYLLFPFL

The HST enzyme may have a mutation at position 280 of SEQ ID NO:14. The mutation may be a substitution of F280. The substitution may be a non-conservative mutation. F280 may be substituted with an aliphatic amino acid residue. For example, the mutation may be F280I. For example, the mutation may be F280L. For example, there is provided herein a nucleic acid molecule encoding an HST enzyme and an HST enzyme that include an amino acid sequence according to Arabidopsis HSTF280I:

(SEQ ID NO: 30)
MELSISQSPRVRFSSLAPRFLAASHHHRPSVHLAGKFISLPRDVRFTS
LSTSRMRILAVALTFKSRCVYVNYEIPKDQILVGAAESDDPVLDRIAR
FQNACWRFLRPHTIRGTALGSTALVTRALIENTHLIKWSLVLKALSGL
LALICGNGYIVGINQIYDIGIDKVNKPYLPIAAGDLSVQSAWLLVIFF
AIAGLLVVGFNFGPFITSLYSLGLFLGTIYSVPPLRMKRFPVAAFLII
ATVRGFLLNFGVYHATRAALGLPFQWSAPVAFITSFVTLIALVIAITK
DLPDVEGDRKFQISTLATKLGVRNIAFLGSGLLLVNYVSAISLAFYMP
QVFRGSLMIPAHVILASGLIFQTWVLEKANYTKEAISGYYRFIWNLFY
AEYLLFPFL

For example, there is provided herein a nucleic acid molecule encoding an HST enzyme and an HST enzyme that include an amino acid sequence according to Arabidopsis HST F280L:

(SEQ ID NO: 31)
MELSISQSPRVRFSSLAPRFLAASHHHRPSVHLAGKFISLPRDVRFTS
LSTSRMRILAVALTFKSRCVYVNYEIPKDQILVGAAESDDPVLDRIAR
FQNACWRFLRPHTIRGTALGSTALVTRALIENTHLIKWSLVLKALSGL
LALICGNGYIVGINQIYDIGIDKVNKPYLPIAAGDLSVQSAWLLVIFF
AIAGLLVVGFNFGPFITSLYSLGLFLGTIYSVPPLRMKRFPVAAFLII
ATVRGFLLNFGVYHATRAALGLPFQWSAPVAFITSFVTLLALVIAITK
DLPDVEGDRKFQISTLATKLGVRNIAFLGSGLLLVNYVSAISLAFYMP
QVFRGSLMIPAHVILASGLIFQTWVLEKANYTKEAISGYYRFIWNLFY
AEYLLFPFL

For example, the HST enzyme may include an amino acid sequence that has a mutation at position 200 of SEQ ID NO:15. For example, the mutation may be a substitution of F200. The substitution may be a non-conservative mutation. F200 may be substituted with an aliphatic amino acid residue. For example, the mutation may be F200I. For example, there is provided herein a nucleic acid molecule encoding an HST enzyme and an HST enzyme that include an amino acid sequence according to Arabidopsis thaliana HST (without transit peptide) F200I:

(SEQ ID NO: 32)
ACSQVGAAESDDPVLDRIARFQNACWRFLRPHTIRGTALGSTALVTRA
LIENTHLIKWSLVLKALSGLLALICGNGYIVGINQIYDIGIDKVNKPY
LPIAAGDLSVQSAWLLVIFFAIAGLLVVGFNFGPFITSLYSLGLFLGT
IYSVPPLRMKRFPVAAFLIIATVRGFLLNFGVYHATRAALGLPFQWSA
PVAFITSIVTLFALVIAITKDLPDVEGDRKFQISTLATKLGVRNIAFL
GSGLLLVNYVSAISLAFYMPQVFRGSLMIPAHVILASGLIFQTWVLEK
ANYTKEAISGYYRFIWNLFYAEYLLFPFL

For example, the HST enzyme may include an amino acid sequence that has a mutation at position 201 of SEQ ID NO: 15. For example, the mutation may be a substitution of V201. The substitution may be a conservative mutation. V201 may be substituted with an aliphatic amino acid residue. For example, the mutation may be V201A. For example, there is provided herein a nucleic acid molecule encoding an HST enzyme and an HST enzyme that include an amino acid sequence according to Arabidopsis thaliana HST (without transit peptide) V201A:

(SEQ ID NO: 33)
ACSQVGAAESDDPVLDRIARFQNACWRFLRPHTIRGTALGSTALVTRA
LIENTHLIKWSLVLKALSGLLALICGNGYIVGINQIYDIGIDKVNKPY
LPIAAGDLSVQSAWLLVIFFAIAGLLVVGFNFGPFITSLYSLGLFLGT
IYSVPPLRMKRFPVAAFLIIATVRGFLLNFGVYHATRAALGLPFQWSA
PVAFITSFATLFALVIAITKDLPDVEGDRKFQISTLATKLGVRNIAFL
GSGLLLVNYVSAISLAFYMPQVFRGSLMIPAHVILASGLIFQTWVLEK
ANYTKEAISGYYRFIWNLFYAEYLLFPFL

For example, the HST enzyme may include an amino acid sequence that has a mutation at position 202 of SEQ ID NO:15. For example, the mutation may be a substitution of T202. The substitution may be a non-conservative mutation. T202 may be substituted with an acidic amino acid residue. For example, the mutation may be T202N. For example, there is provided herein a nucleic acid molecule encoding an HST enzyme and an HST enzyme that include an amino acid sequence according to Arabidopsis thaliana HST (without transit peptide) T202N:

(SEQ ID NO: 34)
ACSQVGAAESDDPVLDRIARFQNACWRFLRPHTIRGTALGSTALVTRA
LIENTHLIKWSLVLKALSGLLALICGNGYIVGINQIYDIGIDKVNKPY
LPIAAGDLSVQSAWLLVIFFAIAGLLVVGFNFGPFITSLYSLGLFLGT
IYSVPPLRMKRFPVAAFLIIATVRGFLLNFGVYHATRAALGLPFQWSA
PVAFITSFVNLFALVIAITKDLPDVEGDRKFQISTLATKLGVRNIAFL
GSGLLLVNYVSAISLAFYMPQVFRGSLMIPAHVILASGLIFQTWVLEK
ANYTKEAISGYYRFIWNLFYAEYLLFPFL

The HST enzyme may have a mutation at position 204 of SEQ ID NO:15. The mutation may be a substitution of F204. The substitution may be a non-conservative mutation. F204 may be substituted with an aliphatic amino acid residue. For example, the mutation may be F204I. For example, the mutation may be F204L For example, there is provided herein a nucleic acid molecule encoding an HST enzyme and an HST enzyme that include an amino acid sequence according to Arabidopsis thaliana HST (without transit peptide) F204I:

(SEQ ID NO: 35)
ACSQVGAAESDDPVLDRIARFQNACWRFLRPHTIRGTALGSTALVTRA
LIENTHLIKWSLVLKALSGLLALICGNGYIVGINQIYDIGIDKVNKPY
LPIAAGDLSVQSAWLLVIFFAIAGLLVVGFNFGPFITSLYSLGLFLGT
IYSVPPLRMKRFPVAAFLIIATVRGFLLNFGVYHATRAALGLPFQWSA
PVAFITSFVTLIALVIAITKDLPDVEGDRKFQISTLATKLGVRNIAFL
GSGLLLVNYVSAISLAFYMPQVFRGSLMIPAHVILASGLIFQTWVLEK
ANYTKEAISGYYRFIWNLFYAEYLLFPFL

For example, there is provided herein a nucleic acid molecule encoding an HST enzyme and an HST enzyme that include an amino acid sequence according to Arabidopsis thaliana HST (without transit peptide) F204L:

(SEQ ID NO: 36)
ACSQVGAAESDDPVLDRIARFQNACWRFLRPHTIRGTALGSTALVTRA
LIENTHLIKWSLVLKALSGLLALICGNGYIVGINQIYDIGIDKVNKPY
LPIAAGDLSVQSAWLLVIFFAIAGLLVVGFNFGPFITSLYSLGLFLGT
IYSVPPLRMKRFPVAAFLIIATVRGFLLNFGVYHATRAALGLPFQWSA
PVAFITSFVTLLALVIAITKDLPDVEGDRKFQISTLATKLGVRNIAFL
GSGLLLVNYVSAISLAFYMPQVFRGSLMIPAHVILASGLIFQTWVLEK
ANYTKEAISGYYRFIWNLFYAEYLLFPFL

For example, the HST enzyme may have a mutation at position 269 of SEQ ID NO:16. For example, the mutation may be a substitution of F269. The substitution may be a non-conservative mutation. F269 may be substituted with an aliphatic amino acid residue. For example, the mutation may be F269I. For example, there is provided herein a nucleic acid molecule encoding an HST enzyme and an HST enzyme that include an amino acid sequence according to Arabidopsis thaliana HST (with transit peptide) F269I:

(SEQ ID NO: 37)
MELSISQSPRVRFSSLAPRFLAASHHHRPSVHLAGKFISLPRDVRFTS
LSTSRMRSKFVSTNYRKISIRACSQVGAAESDDPVLDRIARFQNACWR
FLRPHTIRGTALGSTALVTRALIENTHLIKWSLVLKALSGLLALICGN
GYIVGINQIYDIGIDKVNKPYLPIAAGDLSVQSAWLLVIFFAIAGLLW
VGFNFGPFITSLYSLGLFLGTIYSVPPLRMKRFPVAAFLIIATVRGFL
LNFGVYHATRAALGLPFQWSAPVAFITSIVTLFALVIAITKDLPDVEG
DRKFQISTLATKLGVRNIAFLGSGLLLVNYVSAISLAFYMPQVFRGSL
MIPAHVILASGLIFQTWVLEKANYTKEAISGYYRFIWNLFYAEYLLFP
FL

The HST enzyme may have a mutation at position 270 of SEQ ID NO: 16. The mutation may be a substitution of V270. The substitution may be a conservative mutation. V270 may be substituted with an aliphatic amino acid residue. For example, the mutation may be V270A. For example, there is provided herein a nucleic acid molecule encoding an HST enzyme and an HST enzyme that include an amino acid sequence according to Arabidopsis thaliana HST (with transit peptide) V270A:

(SEQ ID NO: 38)
MELSISQSPRVRFSSLAPRFLAASHHHRPSVHLAGKFISLPRDVRFTS
LSTSRMRSKFVSTNYRKISIRACSQVGAAESDDPVLDRIARFQNACWR
FLRPHTIRGTALGSTALVTRALIENTHLIKWSLVLKALSGLLALICGN
GYIVGINQIYDIGIDKVNKPYLPIAAGDLSVQSAWLLVIFFAIAGLLW
VGFNFGPFITSLYSLGLFLGTIYSVPPLRMKRFPVAAFLIIATVRGFL
LNFGVYHATRAALGLPFQWSAPVAFITSFATLFALVIAITKDLPDVEG
DRKFQISTLATKLGVRNIAFLGSGLLLVNYVSAISLAFYMPQVFRGSL
MIPAHVILASGLIFQTWVLEKANYTKEAISGYYRFIWNLFYAEYLLFP
FL

The HST enzyme may have a mutation at position 271 of SEQ ID NO:16. The mutation may be a substitution of T271. The substitution may be a non-conservative mutation. T271 may be substituted with an acidic amino acid residue. For example, the mutation may be T271N. For example, there is provided herein a nucleic acid molecule encoding an HST enzyme and an HST enzyme that include an amino acid sequence according to Arabidopsis thaliana HST (with transit peptide) T271N:

(SEQ ID NO: 39)
MELSISQSPRVRFSSLAPRFLAASHHHRPSVHLAGKFISLPRDVRFTS
LSTSRMRSKFVSTNYRKISIRACSQVGAAESDDPVLDRIARFQNACWR
FLRPHTIRGTALGSTALVTRALIENTHLIKWSLVLKALSGLLALICGN
GYIVGINQIYDIGIDKVNKPYLPIAAGDLSVQSAWLLVIFFAIAGLLV
VGFNFGPFITSLYSLGLFLGTIYSVPPLRMKRFPVAAFLIIATVRGFL
LNFGVYHATRAALGLPFQWSAPVAFITSFVNLFALVIAITKDLPDVEG
DRKFQISTLATKLGVRNIAFLGSGLLLVNYVSAISLAFYMPQVFRGSL
MIPAHVILASGLIFQTWVLEKANYTKEAISGYYRFIWNLFYAEYLLFP
FL

The HST enzyme may have a mutation at position 273 of SEQ ID NO:16. The mutation may be a substitution of F273. The substitution may be a non-conservative mutation. F273 may be substituted with an aliphatic amino acid residue. For example, the mutation may be F273I. For example, the mutation may be F273L. For example, there is provided herein a nucleic acid molecule encoding an HST enzyme and an HST enzyme that include an amino acid sequence according to Arabidopsis thaliana HST (with transit peptide) F273I:

(SEQ ID NO: 40)
MELSISQSPRVRFSSLAPRFLAASHHHRPSVHLAGKFISLPRDVRFTS
LSTSRMRSKFVSTNYRKISIRACSQVGAAESDDPVLDRIARFQNACWR
FLRPHTIRGTALGSTALVTRALIENTHLIKWSLVLKALSGLLALICGN
GYIVGINQIYDIGIDKVNKPYLPIAAGDLSVQSAWLLVIFFAIAGLLV
VGFNFGPFITSLYSLGLFLGTIYSVPPLRMKRFPVAAFLIIATVRGFL
LNFGVYHATRAALGLPFQWSAPVAFITSFVTLIALVIAITKDLPDVEG
DRKFQISTLATKLGVRNIAFLGSGLLLVNYVSAISLAFYMPQVFRGSL
MIPAHVILASGLIFQTWVLEKANYTKEAISGYYRFIWNLFYAEYLLFP
FL

For example, there is provided herein a nucleic acid molecule encoding an HST enzyme and an HST enzyme that include an amino acid sequence according to Arabidopsis thaliana HST (with transit peptide) F273L:

(SEQ ID NO: 41)
MELSISQSPRVRFSSLAPRFLAASHHHRPSVHLAGKFISLPRDVRFTS
LSTSRMRSKFVSTNYRKISIRACSQVGAAESDDPVLDRIARFQNACWR
FLRPHTIRGTALGSTALVTRALIENTHLIKWSLVLKALSGLLALICGN
GYIVGINQIYDIGIDKVNKPYLPIAAGDLSVQSAWLLVIFFAIAGLLW
VGFNFGPFITSLYSLGLFLGTIYSVPPLRMKRFPVAAFLIIATVRGFL
LNFGVYHATRAALGLPFQWSAPVAFITSFVTLLALVIAITKDLPDVEG
DRKFQISTLATKLGVRNIAFLGSGLLLVNYVSAISLAFYMPQVFRGSL
MIPAHVILASGLIFQTWVLEKANYTKEAISGYYRFIWNLFYAEYLLFP
FL

The HST enzyme may have a mutation at position 199 of SEQ ID NO:17. For example, the mutation may be a substitution of F199. The substitution may be a non-conservative mutation. F199 may be substituted with an aliphatic amino acid residue. For example, the mutation may be F199I. For example, there is provided herein a nucleic acid molecule encoding an HST enzyme and an HST enzyme that include an amino acid sequence according to Oryza sativa HST (without transit peptide) F199I:

(SEQ ID NO 42)
VCSQAGAAGPAPLSKTLSDLKDSCWRFLRPHTIRGTALGSIALVARAL
IENPQLINWWLVFKAFYGLVALICGNGYIVGINQIYDIRIDKVNKPYL
PIAAGDLSVQTAWLLVVLFAAAGFSIVVTNFGPFITSLYCLGLFLGTI
YSVPPFRLKRYPVAAFLIIATVRGFLLNFGVYYATRAALGLTFQWSSP
VAFITCIVTLFALVIAITKDLPDVEGDRKYQISTLATKLGVRNIAFLG
SGLLIANYVAAIAVAFLMPQAFRRTVMVPVHAALAVGIIFQTWVLEQA
KYTKDAISQYYRFIWNLFYAEYIFFPLI

The HST enzyme may have a mutation at position 200 of SEQ ID NO:17. The mutation may be a substitution of V200. The substitution may be a conservative mutation. V200 may be substituted with an aliphatic amino acid residue. For example, the mutation may be V200A. For example, there is provided herein a nucleic acid molecule encoding an HST enzyme and an HST enzyme that include an amino acid sequence according to Oryza sativa HST (without transit peptide) V200A:

(SEQ ID NO: 43)
VCSQAGAAGPAPLSKTLSDLKDSCWRFLRPHTIRGTALGSIALVA
RALIENPQLINWWLVFKAFYGLVALICGNGYIVGINQIYDIRIDK
VNKPYLPIAAGDLSVQTAWLLVVLFAAAGFSIVVTNFGPFITSLY
CLGLFLGTIYSVPPFRLKRYPVAAFLIIATVRGFLLNFGVYYATR
AALGLTFQWSSPVAFITCFATLFALVIAITKDLPDVEGDRKYQIS
TLATKLGVRNIAFLGSGLLIANYVAAIAVAFLMPQAFRRTVMVPV
HAALAVGIIFQTWVLEQAKYTKDAISQYYRFIWNLFYAEYIFFPL
I

The HST enzyme may have a mutation at position 201 of SEQ ID NO:17. The mutation may be a substitution of T201. The substitution may be a non-conservative mutation. T201 may be substituted with an acidic amino acid residue. For example, the mutation may be T201N. For example, there is provided herein a nucleic acid molecule encoding an HST enzyme and an HST enzyme that include an amino acid sequence according to Oryza sativa HST (without transit peptide) T201N:

(SEQ ID NO: 44)
VCSQAGAAGPAPLSKTLSDLKDSCWRFLRPHTIRGTALGSIALVA
RALIENPQLINWWLVFKAFYGLVALICGNGYIVGINQIYDIRIDK
VNKPYLPIAAGDLSVQTAWLLVVLFAAAGFSIVVTNFGPFITSLY
CLGLFLGTIYSVPPFRLKRYPVAAFLIIATVRGFLLNFGVYYATR
AALGLTFQWSSPVAFITCFVNLFALVIAITKDLPDVEGDRKYQIS
TLATKLGVRNIAFLGSGLLIANYVAAIAVAFLMPQAFRRTVMVPV
HAALAVGIIFQTWVLEQAKYTKDAISQYYRFIWNLFYAEYIFFPL
I

The HST enzyme may have a mutation at position 203 of SEQ ID NO:17. The mutation may be a substitution of F203. The substitution may be a non-conservative mutation. F203 may be substituted with an acidic amino acid residue. For example, the mutation may be F203I. For example, the mutation may be F203L. For example, there is provided herein a nucleic acid molecule encoding an HST enzyme and an HST enzyme that include an amino acid sequence according to Oryza sativa HST (without transit peptide) F203I:

(SEQ ID NO: 45)
VCSQAGAAGPAPLSKTLSDLKDSCWRFLRPHTIRGTALGSIALVA
RALIENPQLINWWLVFKAFYGLVALICGNGYIVGINQIYDIRIDK
VNKPYLPIAAGDLSVQTAWLLVVLFAAAGFSIVVTNFGPFITSLY
CLGLFLGTIYSVPPFRLKRYPVAAFLIIATVRGFLLNFGVYYATR
AALGLTFQWSSPVAFITCFVTLIALVIAITKDLPDVEGDRKYQIS
TLATKLGVRNIAFLGSGLLIANYVAAIAVAFLMPQAFRRTVMVPV
HAALAVGIIFQTWVLEQAKYTKDAISQYYRFIWNLFYAEYIFFPL
I

For example, there is provided herein a nucleic acid molecule encoding an HST enzyme and an HST enzyme that include an amino acid sequence according to S Oryza sativa HST (without transit peptide) F203L:

(SEQ ID NO: 46)
VCSQAGAAGPAPLSKTLSDLKDSCWRFLRPHTIRGTALGSIALVA
RALIENPQLINWWLVFKAFYGLVALICGNGYIVGINQIYDIRIDK
VNKPYLPIAAGDLSVQTAWLLVVLFAAAGFSIVVTNFGPFITSLY
CLGLFLGTIYSVPPFRLKRYPVAAFLIIATVRGFLLNFGVYYATR
AALGLTFQWSSPVAFITCFVTLLALVIAITKDLPDVEGDRKYQIS
TLATKLGVRNIAFLGSGLLIANYVAAIAVAFLMPQAFRRTVMVPV
HAALAVGIIFQTWVLEQAKYTKDAISQYYRFIWNLFYAEYIFFPL
I

The HST enzyme may have a mutation at position 262 of SEQ ID NO: 18. For example, the mutation may be a substitution of F262. The substitution may be a non-conservative mutation. F262 may be substituted with an aliphatic amino acid residue. For example, the mutation may be F262I. For example, there is provided herein a nucleic acid molecule encoding an HST enzyme and an HST enzyme that include an amino acid sequence according to Oryza sativa HST (with transit peptide) F262I:

(SEQ ID NO 47)
MASLASPPLPCRAAATASRSGRPAPRLLGPPPPPASPLLSSASAR
FPRAPCNAARWSRRDAVRVCSQAGAAGPAPLSKTLSDLKDSCWRF
LRPHTIRGTALGSIALVARALIENPQLINWWLVFKAFYGLVALIC
GNGYIVGINQIYDIRIDKVNKPYLPIAAGDLSVQTAWLLVVLFAA
AGFSIWVTNFGPFITSLYCLGLFLGTIYSVPPFRLKRYPVAAFLI
IATVRGFLLNFGVYYATRAALGLTFQWSSPVAFITCIVTLFALVI
AITKDLPDVEGDRKYQISTLATKLGVRNIAFLGSGLLIANYVAAI
AVAFLMPQAFRRTVMVPVHAALAVGIIFQTWVLEQAKYTKDAISQ
YYRFIWNLFYAEYIFFPLI

The HST enzyme may have a mutation at position 263 of SEQ ID NO: 18. The mutation may be a substitution of V263. The substitution may be a conservative mutation. V263 may be substituted with an aliphatic amino acid residue. For example, the mutation may be V263A. For example, there is provided herein a nucleic acid molecule encoding an HST enzyme and an HST enzyme that include an amino acid sequence according to Oryza sativa HST (with transit peptide) V263A:

(SEQ ID NO: 48)
MASLASPPLPCRAAATASRSGRPAPRLLGPPPPPASPLLSSASAR
FPRAPCNAARWSRRDAVRVCSQAGAAGPAPLSKTLSDLKDSCWRF
LRPHTIRGTALGSIALVARALIENPQLINWWLVFKAFYGLVALIC
GNGYIVGINQIYDIRIDKVNKPYLPIAAGDLSVQTAWLLVVLFAA
AGFSIWVTNFGPFITSLYCLGLFLGTIYSVPPFRLKRYPVAAFLI
IATVRGFLLNFGVYYATRAALGLTFQWSSPVAFITCFATLFALVI
AITKDLPDVEGDRKYQISTLATKLGVRNIAFLGSGLLIANYVAAI
AVAFLMPQAFRRTVMVPVHAALAVGIIFQTWVLEQAKYTKDAISQ
YYRFIWNLFYAEYIFFPLI

The HST enzyme may have a mutation at position 264 of SEQ ID NO: 18. The mutation may be a substitution of T264. The substitution may be a non-conservative mutation. T264 may be substituted with an acidic amino acid residue. For example, the mutation may be T264N. For example, there is provided herein a nucleic acid molecule encoding an HST enzyme and an HST enzyme that include an amino acid sequence according to Oryza sativa HST (with transit peptide) T264N:

(SEQ ID NO: 49)
MASLASPPLPCRAAATASRSGRPAPRLLGPPPPPASPLLSSASAR
FPRAPCNAARWSRRDAVRVCSQAGAAGPAPLSKTLSDLKDSCWRF
LRPHTIRGTALGSIALVARALIENPQLINWWLVFKAFYGLVALIC
GNGYIVGINQIYDIRIDKVNKPYLPIAAGDLSVQTAWLLVVLFAA
AGFSIWVTNFGPFITSLYCLGLFLGTIYSVPPFRLKRYPVAAFLI
IATVRGFLLNFGVYYATRAALGLTFQWSSPVAFITCFVNLFALVI
AITKDLPDVEGDRKYQISTLATKLGVRNIAFLGSGLLIANYVAAI
AVAFLMPQAFRRTVMVPVHAALAVGIIFQTWVLEQAKYTKDAISQ
YYRFIWNLFYAEYIFFPLI

The HST enzyme may have a mutation at position 266 of SEQ ID NO: 18. The mutation may be a substitution of F266. The substitution may be a non-conservative mutation. F266 may be substituted with an acidic amino acid residue. For example, the mutation may be F266I. For example, the mutation may be F266L. For example, there is provided herein a nucleic acid molecule encoding an HST enzyme and an HST enzyme that include an amino acid sequence according to Oryza sativa HST (with transit peptide) F266I:

(SEQ ID NO: 50)
MASLASPPLPCRAAATASRSGRPAPRLLGPPPPPASPLLSSASAR
FPRAPCNAARWSRRDAVRVCSQAGAAGPAPLSKTLSDLKDSCWRF
LRPHTIRGTALGSIALVARALIENPQLINWWLVFKAFYGLVALIC
GNGYIVGINQIYDIRIDKVNKPYLPIAAGDLSVQTAWLLVVLFAA
AGFSIWTNFGPFITSLYCLGLFLGTIYSVPPFRLKRYPVAAFLII
ATVRGFLLNFGVYYATRAALGLTFQWSSPVAFITCFVTLIALVIA
ITKDLPDVEGDRKYQISTLATKLGVRNIAFLGSGLLIANYVAAIA
VAFLMPQAFRRTVMVPVHAALAVGIIFQTWVLEQAKYTKDAISQY
YRFIWNLFYAEYIFFPLI

For example, there is provided herein a nucleic acid molecule encoding an HST enzyme that comprises an amino acid sequence according to Oryza sativa HST (with transit peptide) F266L:

(SEQ ID NO: 51)
MASLASPPLPCRAAATASRSGRPAPRLLGPPPPPASPLLSSASAR
FPRAPCNAARWSRRDAVRVCSQAGAAGPAPLSKTLSDLKDSCWRF
LRPHTIRGTALGSIALVARALIENPQLINWWLVFKAFYGLVALIC
GNGYIVGINQIYDIRIDKVNKPYLPIAAGDLSVQTAWLLVVLFAA
AGFSIWVTNFGPFITSLYCLGLFLGTIYSVPPFRLKRYPVAAFLI
IATVRGFLLNFGVYYATRAALGLTFQWSSPVAFITCFVTLLALVI
AITKDLPDVEGDRKYQISTLATKLGVRNIAFLGSGLLIANYVAAI
AVAFLMPQAFRRTVMVPVHAALAVGIIFQTWVLEQAKYTKDAISQ
YYRFIWNLFYAEYIFFPLI

For example, there is provided herein a nucleic acid molecule encoding an HST enzyme that comprises a mutation at position 198 of SEQ ID NO:19. For example, the mutation may be a substitution of F198. The substitution may be a non-conservative mutation. F198 may be substituted with an aliphatic amino acid residue. For example, the mutation may be F198I. For example, there is provided herein a nucleic acid molecule encoding an HST enzyme that comprises an amino acid sequence according to Chlorella fusca (without transit peptide) F198I:

(SEQ ID NO: 52)
SSTYSGPDGWSGDGSQGSFLSAFWRFLRPHTIRGTILGTSAVVTK
VVLSNMEAIDWGLLPRALMGLVALLCGNGYIVGINQIYDVDIDAV
NKPFLPVAAGDMSPGTAWVLVLALAAAGLGIVATNFGNLITGLYG
FGLLLGTVYSVPPLRLKRFAVPAFLIIATVRGFLLNFGVYHATRA
ALQLPFEWSPAILFITCIVTMFATVIAITKDLPDIEGDKANNIST
FATRLGVKNVSLLGVGMLLLNYVMAGVFALKYSSYFNVPVMLGAH
ALLALVLILRTTKLAAAGYTQQAVISFYRWIWNLFYSEYLVYVLI

For example, there is provided herein a nucleic acid molecule encoding an HST enzyme that comprises a mutation at position 199 of SEQ ID NO:19. The mutation may be a substitution of V199. The substitution may be a conservative mutation. V199 may be substituted with an aliphatic amino acid residue. For example, the mutation may be V199A. For example, there is provided herein a nucleic acid molecule encoding an HST enzyme that comprises an amino acid sequence according to Chlorella fusca (without transit peptide) V199A:

(SEQ ID NO: 53)
SSTYSGPDGWSGDGSQGSFLSAFWRFLRPHTIRGTILGTSAVVTK
VVLSNMEAIDWGLLPRALMGLVALLCGNGYIVGINQIYDVDIDAV
NKPFLPVAAGDMSPGTAWVLVLALAAAGLGIVATNFGNLITGLYG
FGLLLGTVYSVPPLRLKRFAVPAFLIIATVRGFLLNFGVYHATRA
ALQLPFEWSPAILFITCFATMFATVIAITKDLPDIEGDKANNIST
FATRLGVKNVSLLGVGMLLLNYVMAGVFALKYSSYFNVPVMLGAH
ALLALVLILRTTKLAAAGYTQQAVISFYRWIWNLFYSEYLVYVLI

For example, there is provided herein a nucleic acid molecule encoding an HST enzyme that comprises a mutation at position 200 of SEQ ID NO:19. The mutation may be a substitution of T200. The substitution may be a non-conservative mutation. T200 may be substituted with an acidic amino acid residue. For example, the mutation may be T200N. For example, there is provided herein a nucleic acid molecule encoding an HST enzyme that comprises an amino acid sequence according to Chlorella fusca (without transit peptide) T200N:

(SEQ ID NO: 54)
SSTYSGPDGWSGDGSQGSFLSAFWRFLRPHTIRGTILGTSAVVTK
VVLSNMEAIDWGLLPRALMGLVALLCGNGYIVGINQIYDVDIDAV
NKPFLPVAAGDMSPGTAWVLVLALAAAGLGIVATNFGNLITGLYG
FGLLLGTVYSVPPLRLKRFAVPAFLIIATVRGFLLNFGVYHATRA
ALQLPFEWSPAILFITCFVNMFATVIAITKDLPDIEGDKANNIST
FATRLGVKNVSLLGVGMLLLNYVMAGVFALKYSSYFNVPVMLGAH
ALLALVLILRTTKLAAAGYTQQAVISFYRWIWNLFYSEYLVYVLI

For example, there is provided herein a nucleic acid molecule encoding an HST enzyme that comprises a mutation at positions 201 and 202 of SEQ ID NO: 19. The mutation may be a substitution of M201 and F202. For example, the mutation may be M20L and F202I. For example, there is provided herein a nucleic acid molecule encoding an HST enzyme that comprises an amino acid sequence according to Chlorella fusca (without transit peptide) M201L F202I:

(SEQ ID NO: 55)
SSTYSGPDGWSGDGSQGSFLSAFWRFLRPHTIRGTILGTSAVVTK
VVLSNMEAIDWGLLPRALMGLVALLCGNGYIVGINQIYDVDIDAV
NKPFLPVAAGDMSPGTAWVLVLALAAAGLGIVATNFGNLITGLYG
FGLLLGTVYSVPPLRLKRFAVPAFLIIATVRGFLLNFGVYHATRA
ALQLPFEWSPAILFITCFVTLIATVIAITKDLPDIEGDKANNIST
FATRLGVKNVSLLGVGMLLLNYVMAGVFALKYSSYFNVPVMLGAH
ALLALVLILRTTKLAAAGYTQQAVISFYRWIWNLFYSEYLVYVLI

For example, there is provided herein a nucleic acid molecule encoding an HST enzyme that comprises a mutation at position 202 of SEQ ID NO:19. The mutation may be a substitution of F202. The substitution may be a non-conservative mutation. F202 may be substituted with an aliphatic amino acid residue. For example, the mutation may be F202I. For example, the mutation may be F202I. For example, there is provided herein a nucleic acid molecule encoding an HST enzyme that comprises an amino acid sequence according to Chlorella fusca (without transit peptide) F202I:

(SEQ ID NO: 56)
SSTYSGPDGWSGDGSQGSFLSAFWRFLRPHTIRGTILGTSAVVTK
VVLSNMEAIDWGLLPRALMGLVALLCGNGYIVGINQIYDVDIDAV
NKPFLPVAAGDMSPGTAWVLVLALAAAGLGIVATNFGNLITGLYG
FGLLLGTVYSVPPLRLKRFAVPAFLIIATVRGFLLNFGVYHATRA
ALQLPFEWSPAILFITCFVTMIATVIAITKDLPDIEGDKANNIST
FATRLGVKNVSLLGVGMLLLNYVMAGVFALKYSSYFNVPVMLGAH
ALLALVLILRTTKLAAAGYTQQAVISFYRWIWNLFYSEYLVYVLI

For example, there is provided herein a nucleic acid molecule encoding an HST enzyme that comprises a mutation at position 202 of SEQ ID NO:19. The mutation may be a substitution of F202. The substitution may be a non-conservative mutation. F202 may be substituted with an acidic amino acid residue. For example, the mutation may be F202I. For example, the mutation may be F202L. For example, there is provided herein a nucleic acid molecule encoding an HST enzyme that comprises an amino acid sequence according to Chlorella fusca (without transit peptide) F202L:

(SEQ ID NO: 57)
SSTYSGPDGWSGDGSQGSFLSAFWRFLRPHTIRGTILGTSAVVTK
VVLSNMEAIDWGLLPRALMGLVALLCGNGYIVGINQIYDVDIDAV
NKPFLPVAAGDMSPGTAWVLVLALAAAGLGIVATNFGNLITGLYG
FGLLLGTVYSVPPLRLKRFAVPAFLIIATVRGFLLNFGVYHATRA
ALQLPFEWSPAILFITCFVTMLATVIAITKDLPDIEGDKANNIST
FATRLGVKNVSLLGVGMLLLNYVMAGVFALKYSSYFNVPVMLGAH
ALLALVLILRTTKLAAAGYTQQAVISFYRWIWNLFYSEYLVYVLI

For example, there is provided herein a nucleic acid molecule encoding an HST enzyme that comprises a mutation at position 275 of SEQ ID NO:20. For example, the mutation may be a substitution of F275. The substitution may be a non-conservative mutation. F275 may be substituted with an aliphatic amino acid residue. For example, the mutation may be F275I For example, there is provided herein a nucleic acid molecule encoding an HST enzyme that comprises an amino acid sequence according to Chlorella fusca HST F275I (Cf-496-19):

(SEQ ID NO: 58)
MLSSGQNQVQQSLGRHKSSRWSCSHAFQPSRTSQWPRLLPGLHSKVQQQH
HLHQLPQQLQQHQLQQPERLVSTQAASSSTYSGPDGWSGDGSQGSFLSAF
WRFLRPHTIRGTILGTSAVVTKVVLSNMEAIDWGLLPRALMGLVALLCGN
GYIVGINQIYDVDIDAVNKPFLPVAAGDMSPGTAWVLVLALAAAGLGIVA
TNFGNLITGLYGFGLLLGTVYSVPPLRLKRFAVPAFLIIATVRGFLLNFG
VYHATRAALQLPFEWSPAILFITCIVTMFATVIAITKDLPDIEGDKANNI
STFATRLGVKNVSLLGVGMLLLNYVMAGVFALKYSSYFNVPVMLGAHALL
ALVLILRTTKLAAAGYTQQAVISFYRWIWNLFYSEYLVYVLI

For example, there is provided herein a nucleic acid molecule encoding an HST enzyme that comprises a mutation at position 276 of SEQ ID NO:20. The mutation may be a substitution of V276. The substitution may be a conservative mutation. V276 may be substituted with an aliphatic amino acid residue. For example, the mutation may be V276A. For example, there is provided herein a nucleic acid molecule encoding an HST enzyme that comprises an amino acid sequence according to Chlorella fusca HST V276A (Cf-496-28):

(SEQ ID NO: 59)
MLSSGQNQVQQSLGRHKSSRWSCSHAFQPSRTSQWPRLLPGLHSKVQQQH
HLHQLPQQLQQHQLQQPERLVSTQAASSSTYSGPDGWSGDGSQGSFLSAF
WRFLRPHTIRGTILGTSAVVTKVVLSNMEAIDWGLLPRALMGLVALLCGN
GYIVGINQIYDVDIDAVNKPFLPVAAGDMSPGTAWVLVLALAAAGLGIVA
TNFGNLITGLYGFGLLLGTVYSVPPLRLKRFAVPAFLIIATVRGFLLNFG
VYHATRAALQLPFEWSPAILFITCFATMFATVIAITKDLPDIEGDKANNI
STFATRLGVKNVSLLGVGMLLLNYVMAGVFALKYSSYFNVPVMLGAHALL
ALVLILRTTKLAAAGYTQQAVISFYRWIWNLFYSEYLVYVLI

For example, there is provided herein a nucleic acid molecule encoding an HST enzyme that comprises a mutation at position 277 of SEQ ID NO:20. The mutation may be a substitution of T277. The substitution may be a non-conservative mutation. T277 may be substituted with an acidic amino acid residue. For example, the mutation may be T277N. For example, there is provided herein a nucleic acid molecule encoding an HST enzyme that comprises an amino acid sequence according to Chlorella fusca HST T277N (Cf-496-15):

(SEQ ID NO: 60)
MLSSGQNQVQQSLGRHKSSRWSCSHAFQPSRTSQWPRLLPGLHSKVQQQH
HLHQLPQQLQQHQLQQPERLVSTQAASSSTYSGPDGWSGDGSQGSFLSAF
WRFLRPHTIRGTILGTSAVVTKVVLSNMEAIDWGLLPRALMGLVALLCGN
GYIVGINQIYDVDIDAVNKPFLPVAAGDMSPGTAWVLVLALAAAGLGIVA
TNFGNLITGLYGFGLLLGTVYSVPPLRLKRFAVPAFLIIATVRGFLLNFG
VYHATRAALQLPFEWSPAILFITCFVNMFATVIAITKDLPDIEGDKANNI
STFATRLGVKNVSLLGVGMLLLNYVMAGVFALKYSSYFNVPVMLGAHALL
ALVLILRTTKLAAAGYTQQAVISFYRWIWNLFYSEYLVYVLI

For example, there is provided herein a nucleic acid molecule encoding an HST enzyme that comprises a mutation at positions 278 and 279 of SEQ ID NO: 20. The mutation may be a substitution of M278 and F279. For example, the mutation may be M278L and F279I. For example, there is provided herein a nucleic acid molecule encoding an HST enzyme and an HST enzyme that include an amino acid sequence according to Chlorella fusca HST M278L F279I (Cf-496-8):

(SEQ ID NO: 61)
MLSSGQNQVQQSLGRHKSSRWSCSHAFQPSRTSQWPRLLPGLHSKVQQQH
HLHQLPQQLQQHQLQQPERLVSTQAASSSTYSGPDGWSGDGSQGSFLSAF
WRFLRPHTIRGTILGTSAVVTKVVLSNMEAIDWGLLPRALMGLVALLCGN
GYIVGINQIYDVDIDAVNKPFLPVAAGDMSPGTAWVLVLALAAAGLGIVA
TNFGNLITGLYGFGLLLGTVYSVPPLRLKRFAVPAFLIIATVRGFLLNFG
VYHATRAALQLPFEWSPAILFITCFVTLIATVIAITKDLPDIEGDKANNI
STFATRLGVKNVSLLGVGMLLLNYVMAGVFALKYSSYFNVPVMLGAHALL
ALVLILRTTKLAAAGYTQQAVISFYRWIWNLFYSEYLVYVLI

For example, there is provided herein a nucleic acid molecule encoding an HST enzyme that comprises a mutation at position 279 of SEQ ID NO:20. The mutation may be a substitution of F279. The substitution may be a non-conservative mutation. F279 may be substituted with an acidic amino acid residue. For example, the mutation may be F279I. For example, the mutation may be F279I. For example, there is provided herein a nucleic acid molecule encoding an HST enzyme that include comprises an amino acid sequence according to Chlorella fusca HST F279I (Cf-496-8):

(SEQ ID NO: 62)
MLSSGQNQVQQSLGRHKSSRWSCSHAFQPSRTSQWPRLLPGLHSKVQQQH
HLHQLPQQLQQHQLQQPERLVSTQAASSSTYSGPDGWSGDGSQGSFLSAF
WRFLRPHTIRGTILGTSAVVTKVVLSNMEAIDWGLLPRALMGLVALLCGN
GYIVGINQIYDVDIDAVNKPFLPVAAGDMSPGTAWVLVLALAAAGLGIVA
TNFGNLITGLYGFGLLLGTVYSVPPLRLKRFAVPAFLIIATVRGFLLNFG
VYHATRAALQLPFEWSPAILFITCFVTMIATVIAITKDLPDIEGDKANNI
STFATRLGVKNVSLLGVGMLLLNYVMAGVFALKYSSYFNVPVMLGAHALL
ALVLILRTTKLAAAGYTQQAVISFYRWIWNLFYSEYLVYVLI

For example, there is provided herein a nucleic acid molecule encoding an HST enzyme comprising a mutation at position 279 of SEQ ID NO:20. The mutation may be a substitution of F279. The substitution may be a non-conservative mutation. F279 may be substituted with an acidic amino acid residue. For example, the mutation may be F280I. For example, the mutation may be F279L. For example, there is provided herein a nucleic acid molecule encoding an HST enzyme that comprises an amino acid sequence according to Chlorella fusca HST F279L (Cf-496-94)

(SEQ ID NO: 63)
MLSSGQNQVQQSLGRHKSSRWSCSHAFQPSRTSQWPRLLPGLHSKVQQQH
HLHQLPQQLQQHQLQQPERLVSTQAASSSTYSGPDGWSGDGSQGSFLSAF
WRFLRPHTIRGTILGTSAVVTKVVLSNMEAIDWGLLPRALMGLVALLCGN
GYIVGINQIYDVDIDAVNKPFLPVAAGDMSPGTAWVLVLALAAAGLGIVA
TNFGNLITGLYGFGLLLGTVYSVPPLRLKRFAVPAFLIIATVRGFLLNFG
VYHATRAALQLPFEWSPAILFITCFVTMLATVIAITKDLPDIEGDKANNI
STFATRLGVKNVSLLGVGMLLLNYVMAGVFALKYSSYFNVPVMLGAHALL
ALVLILRTTKLAAAGYTQQAVISFYRWIWNLFYSEYLVYVLI

For example, there is provided herein a nucleic acid molecule encoding an HST enzyme that comprises a mutation at position 261 of SEQ ID NO:21. For example, the mutation may be a substitution of F261. The substitution may be a non-conservative mutation. F261 may be substituted with an aliphatic amino acid residue. For example, the mutation may be F261I. For example, there is provided herein a nucleic acid molecule encoding an HST enzyme that comprises an amino acid sequence according to Chlorella fusca (with transit peptide) F261I:

(SEQ ID NO: 64)
MLRSSRWSCSHAFQPSRTSQWPRLLPGLHSKVQQQHHLHQLPQQLQQHQL
QQPERLVSTQAASSSTYSGPDGWSGDGSQGSFLSAFWRFLRPHTIRGTIL
GTSAVVTKVVLSNMEAIDWGLLPRALMGLVALLCGNGYIVGINQIYDVDI
DAVNKPFLPVAAGDMSPGTAWVLVLALAAAGLGIVATNFGNLITGLYGFG
LLLGTVYSVPPLRLKRFAVPAFLIIATVRGFLLNFGVYHATRAALQLPFE
WSPAILFITCIVTMFATVIAITKDLPDIEGDKANNISTFATRLGVKNVSL
LGVGMLLLNYVMAGVFALKYSSYFNVPVMLGAHALLALVLILRTTKLAAA
GYTQQAVISFYRWIWNLFYSEYLVYVLI

For example, there is provided herein a nucleic acid molecule encoding an HST enzyme that comprises a mutation at position 262 of SEQ ID NO: 21. The mutation may be a substitution of V262. The substitution may be a conservative mutation. V262 may be substituted with an aliphatic amino acid residue. For example, the mutation may be V262A. For example, there is provided herein a nucleic acid molecule encoding an HST enzyme that comprises an amino acid sequence according to Chlorella fusca (with transit peptide) V262A:

(SEQ ID NO: 65)
MLRSSRWSCSHAFQPSRTSQWPRLLPGLHSKVQQQHHLHQLPQQLQQHQL
QQPERLVSTQAASSSTYSGPDGWSGDGSQGSFLSAFWRFLRPHTIRGTIL
GTSAVVTKVVLSNMEAIDWGLLPRALMGLVALLCGNGYIVGINQIYDVDI
DAVNKPFLPVAAGDMSPGTAWVLVLALAAAGLGIVATNFGNLITGLYGFG
LLLGTVYSVPPLRLKRFAVPAFLIIATVRGFLLNFGVYHATRAALQLPFE
WSPAILFITCFATMFATVIAITKDLPDIEGDKANNISTFATRLGVKNVSL
LGVGMLLLNYVMAGVFALKYSSYFNVPVMLGAHALLALVLILRTTKLAAA
GYTQQAVISFYRWIWNLFYSEYLVYVLI

For example, there is provided herein a nucleic acid molecule encoding an HST enzyme that comprises a mutation at position 263 of SEQ ID NO:21. The mutation may be a substitution of T263. The substitution may be a non-conservative mutation. T263 may be substituted with an acidic amino acid residue. For example, the mutation may be T263N. For example, there is provided herein a nucleic acid molecule encoding an HST enzyme that comprises an amino acid sequence according to Chlorella fusca (with transit peptide) T263N:

(SEQ ID NO: 66)
MLRSSRWSCSHAFQPSRTSQWPRLLPGLHSKVQQQHHLHQLPQQLQQHQL
QQPERLVSTQAASSSTYSGPDGWSGDGSQGSFLSAFWRFLRPHTIRGTIL
GTSAVVTKVVLSNMEAIDWGLLPRALMGLVALLCGNGYIVGINQIYDVDI
DAVNKPFLPVAAGDMSPGTAWVLVLALAAAGLGIVATNFGNLITGLYGFG
LLLGTVYSVPPLRLKRFAVPAFLIIATVRGFLLNFGVYHATRAALQLPFE
WSPAILFITCFVNMFATVIAITKDLPDIEGDKANNISTFATRLGVKNVSL
LGVGMLLLNYVMAGVFALKYSSYFNVPVMLGAHALLALVLILRTTKLAAA
GYTQQAVISFYRWIWNLFYSEYLVYVLI

For example, there is provided herein a nucleic acid molecule encoding an HST enzyme that comprises a mutation at positions 264 and 265 of SEQ ID NO: 21. The mutation may be a substitution of M264 and F265. For example, the mutation may be M264L and F265I. For example, there is provided herein a nucleic acid molecule encoding an HST enzyme that comprises an amino acid sequence according to Chlorella fusca (with transit peptide) M264L F265I:

(SEQ ID NO: 67)
MLRSSRWSCSHAFQPSRTSQWPRLLPGLHSKVQQQHHLHQLPQQLQQHQL
QQPERLVSTQAASSSTYSGPDGWSGDGSQGSFLSAFWRFLRPHTIRGTIL
GTSAVVTKVVLSNMEAIDWGLLPRALMGLVALLCGNGYIVGINQIYDVDI
DAVNKPFLPVAAGDMSPGTAWVLVLALAAAGLGIVATNFGNLITGLYGFG
LLLGTVYSVPPLRLKRFAVPAFLIIATVRGFLLNFGVYHATRAALQLPFE
WSPAILFITCFVTLIATVIAITKDLPDIEGDKANNISTFATRLGVKNVSL
LGVGMLLLNYVMAGVFALKYSSYFNVPVMLGAHALLALVLILRTTKLAAA
GYTQQAVISFYRWIWNLFYSEYLVYVLI

For example, there is provided herein a nucleic acid molecule encoding an HST enzyme that comprises a mutation at position 265 of SEQ ID NO: 21. The mutation may be a substitution of F265. The substitution may be a non-conservative mutation. F265 may be substituted with an acidic amino acid residue. For example, the mutation may be F265I. For example, the mutation may be F265I. For example, there is provided herein a nucleic acid molecule encoding an HST enzyme that comprises an amino acid sequence according to Chlorella fusca (with transit peptide) F265I:

(SEQ ID NO: 68)
MLRSSRWSCSHAFQPSRTSQWPRLLPGLHSKVQQQHHLHQLPQQLQQHQL
QQPERLVSTQAASSSTYSGPDGWSGDGSQGSFLSAFWRFLRPHTIRGTIL
GTSAVVTKVVLSNMEAIDWGLLPRALMGLVALLCGNGYIVGINQIYDVDI
DAVNKPFLPVAAGDMSPGTAWVLVLALAAAGLGIVATNFGNLITGLYGFG
LLLGTVYSVPPLRLKRFAVPAFLIIATVRGFLLNFGVYHATRAALQLPFE
WSPAILFITCFVTMIATVIAITKDLPDIEGDKANNISTFATRLGVKNVSL
LGVGMLLLNYVMAGVFALKYSSYFNVPVMLGAHALLALVLILRTTKLAAA
GYTQQAVISFYRWIWNLFYSEYLVYVLI

For example, there is provided herein a nucleic acid molecule encoding an HST enzyme that comprises a mutation at position 265 of SEQ ID NO:20. The mutation may be a substitution of F265. The substitution may be a non-conservative mutation. F265 may be substituted with an aliphatic amino acid residue. For example, the mutation may be F279L. For example, there is provided herein a nucleic acid molecule encoding an HST enzyme that comprises an amino acid sequence according to Chlorella fusca (with transit peptide) F265L:

(SEQ ID NO: 69)
MLRSSRWSCSHAFQPSRTSQWPRLLPGLHSKVQQQHHLHQLPQQLQQHQL
QQPERLVSTQAASSSTYSGPDGWSGDGSQGSFLSAFWRFLRPHTIRGTIL
GTSAVVTKVVLSNMEAIDWGLLPRALMGLVALLCGNGYIVGINQIYDVDI
DAVNKPFLPVAAGDMSPGTAWVLVLALAAAGLGIVATNFGNLITGLYGFG
LLLGTVYSVPPLRLKRFAVPAFLIIATVRGFLLNFGVYHATRAALQLPFE
WSPAILFITCFVTMLATVIAITKDLPDIEGDKANNISTFATRLGVKNVSL
LGVGMLLLNYVMAGVFALKYSSYFNVPVMLGAHALLALVLILRTTKLAAA
GYTQQAVISFYRWIWNLFYSEYLVYVLI

The HST enzymes or functional fragments thereof provided herein usually comprise a transit peptide. For example, a chloroplast transit peptide. A transit peptide or chloroplast transit peptide refers to a signal sequence in chloroplast interior proteins. In the cytosol, the transit peptide is recognized by cytosolic chaperones such as Hsp70, Hsp90, or factors yet to be identified, which leads to the targeting of preproteins to the chloroplast. Transit peptides may be included in expression cassettes as detailed herein below. The transit peptide may be any naturally occurring or synthetic transit peptide. As detailed herein, the HST enzyme may include its naturally occurring peptide. In other examples, such as for example with reference to the HST enzymes described herein that do not include a transit peptide (e.g. SEQ ID NOs: 13 to 15, 17, 19 and 20 and sequences related thereto) a heterologous transit peptide, i.e. from another HST enzyme and/or from another species, may be added to create a chimeric HST protein.

For example the transit peptides that may be added may be those form other wild-type HST enzymes such as for example Arabidopsis thaliana HST transit peptide:

(SEQ ID NO: 70)
MELSISQSPRVRFSSLAPRFLAASHHHRPSVHLAGKFISLPRDVRFTSLS
TSRMRSKFVSTNYRKISIR

Oryza sativa HST transit peptide:

(SEQ ID NO: 71)
MASLASPPLPCRAAATASRSGRPAPRLLGPPPPPASPLLSSASARFPRAP
CNAARWSRRDAVR

Chlorella fusca transit peptide:

(SEQ ID NO: 72)
MLRSSRWSCSHAFQPSRTSQWPRLLPGLHSKVQQQHHLHQLPQQLQQHQL
QQPERLVSTQAAS

Other examples of transit peptides that may be used with the HST enzymes of the invention are described below in relation to expression cassettes for expressing HST enzymes of the invention.

A transit peptide of the invention may have at least 60% sequence identity to any one of SEQ ID NO: 70 to 72. For example, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 70 to 72.

The nucleic acids encoding an HST enzyme as described herein may be included in an expression cassette. Such expression cassettes may be included in an expression vector. Therefore, there is also provided expression cassettes and expression vectors including a nucleic acid encoding an HST enzyme as described herein.

### Expression Vectors

The expression vectors, which include at least one nucleic acid molecule of the present invention inserted therein may be any vector capable of delivering the nucleic acid molecule into a host or host cell and allowing expression of the nucleic acid molecule to provide a functional HST enzyme as described herein or fragment thereof. Such vectors may contain heterologous nucleic acid sequences, that is nucleic acid sequences that are not naturally found adjacent to nucleic acid molecules of the present invention and that may be derived from a species other than the species from which the nucleic acid molecule(s) are derived. The vector can be either RNA or DNA, either prokaryotic or eukaryotic, and typically is a virus or a plasmid.

A number of vectors suitable for stable transfection of plant cells or for the establishment of transgenic plants have been described in, e.g., Pouwels et al., Cloning Vectors: A Laboratory Manual, 1985, supp 1987; Weissbach and Weissbach, Methods for Plant Molecular Biology. Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. For example the vector may be pBIN 19 (Bevan, Nucl. Acids Res. (1984)).

The expression vector of the invention may include one or more regulatory sequences. For example, the expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal. Such a portion of an expression vector may be referred to as an expression cassette. The expression cassettes may preferably include one or more regulatory sequences that are functional in plants. Thus allowing expression of the nucleic acid encoding an HST enzyme of the invention in a plant.

“Expression cassette” as used herein means a nucleic acid sequence capable of directing expression of a particular nucleic acid sequence in an appropriate host cell, comprising a promoter operably linked to the nucleic acid sequence of interest which is operably linked to termination signal sequences. It also typically comprises sequences required for proper translation of the nucleic acid sequence. The expression cassette comprising the nucleic acid sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event. The expression of the nucleic acid sequence in the expression cassette may be under the control of, for example, a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

The term “regulatory element” or “regulatory sequence” as used herein refers to a nucleic acid that is capable of regulating the transcription and/or translation of an operably linked nucleic acid molecule. Regulatory elements include, but are not limited to, promoters, enhancers, introns, 5′ UTRs, and 3′ UTRs.

Expression cassettes may include in the 5′-3′ direction of transcription, a transcriptional and translational initiation region (e.g., a promoter), a HST nucleic acid sequence of the invention, and a transcriptional and translational termination region (e.g., termination region) functional in plants.

Any promoter can be used in the production of the expression cassettes and vectors including such expression cassettes as described herein. The promoter may be native or analogous, or foreign or heterologous, to the plant host and/or to the HST nucleic acid sequences of the invention. Additionally, the promoter may be a natural sequence or alternatively a synthetic sequence. Where the promoter is “foreign” or “heterologous” to the plant host, it is intended that the promoter is not found in the native plant into which the promoter is introduced. Where the promoter is “foreign” or “heterologous” to the HST encoding nucleic acid molecule of the invention, it is intended that the promoter is not the native or naturally occurring promoter for the operably linked HST nucleic acid molecule of the invention.

While it may be preferable to express the HST nucleic acid molecules of the invention using heterologous promoters, the native promoter sequences may be used in the preparation of the expression cassettes. Such expression cassettes may change expression levels of the HST enzyme in the plant or plant cell. Thus, the phenotype of the plant or plant cell is altered.

Any promoter can be used in the preparation of expression cassettes to control the expression of the HST encoding nucleic acid molecule, such as promoters providing for constitutive, tissue-preferred, inducible, or other promoters for expression in plants. Constitutive promoters include, for example, the core promoter of the Rsyn7 promoter and other constitutive promoters disclosed in WO 99/43 838 and U.S. Pat. No. 6,072,050; the core CaMV 35S promoter (Odell et al. (1985) Nature 313:810-812; rice actin (McElroy et al. (1990) Plant Cell 2:163-171); ubiquitin (Christensen et al. (1989) Plant Mol. Biol. 12:613-632 and Christensen et al. (1992) Plant Mol. Biol. 18:675-689). pEMU (Last et al. (1991) Theor. Appl. Genet. 81:581-588); MAS (Velten et al. (1984) EMBO J. 3:2723-2730), ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and U.S. Pat. No. 6,177,611.

Tissue-preferred promoters can be utilized to direct expression of the HST enzymes of the invention within a particular plant tissue. Such tissue-preferred promoters include, but are not limited to, leaf-preferred promoters, root-preferred promoters, seed-preferred promoters, and stem-preferred promoters. Tissue-preferred promoters include those described in stem-preferred promoters. Tissue-preferred promoters include those described in Yamamoto et al. (1997) Plant J. 12(2):255-268; Kawamata et al. (1997) Plant Cell Physiol. 38(7):792-803; Hansen et al. (1997) Mol Gen Genet. 254(3):337-343, Russell et al. (1997) Transgenic Res. 6(2): 157-163: Rinehart et al. (1996) Plant Physiol. 112(3):1331-1341; Van Camp et al. (1996) Plant Physiol. 1 12(2):525-535; Canevascini et al. (1996) Plant Physiol. 112(2): 513-524; Yamamoto et al. (1994) Plant Cell Physiol. 35(5):773-778; Lam (1994) Results Probl. Cell Differ 20:181-196; Orozco et al (1993) Plant Mol Biol. 23(6); 1129-1138; Matsuoka et al. (1993) Proc Natl. Acad Sci. USA 90(20):9586-9530; and Guevara-Garcia et al. (1993) Plant J. 4(3):495-505.

The expression cassettes may also comprise transcription termination regions Where transcription terminations regions are used, any termination region may be used in the preparation of the expression cassettes. For example, the termination region may be native to the transcriptional initiation region, may be native to the operably linked HST encoding nucleic acid molecule of interest, may be native to the plant host, or may be derived from another source (i.e., foreign or heterologous to the promoter, the HST nucleic acid molecule of interest, the plant host, or any combination thereof). Examples of termination regions that are available for use in the expression cassettes and vectors of the present invention include those from the Ti-plasmid of A. tumefaciens, such as the octopine synthase and nopaline synthase termination regions. See also Guerineau et al. (1991) Mol. Gen. Genet. 262: 141-144, Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

For example, the expression cassette may comprise a Tomato Mosaic Virus (TMV) omega 5′ leader and a HST encoding gene of interest is excised using Xhol/Kpnl and cloned into pBIN 19 behind a double enhanced 35S promoter and ahead of a NOS 3′ transcription terminator.

The nucleic acids may be optimized for increased expression in a transformed plant. That is, the nucleic acids encoding the HST enzymes can be synthesized using plant-preferred codons for improved expression. See, for example, Campbell an Growri (1990) Plant Physiol. 92; 1-11 for a discussion of host-preferred codon usage. Methods are available in the art for synthesizing plant-preferred genes. See, for example U.S. Pat. No. 5,380,831, and U.S. Pat. No. 5,436.391, and Murray et al. (1989) Nucleic Acids Res. 17:477-498, herein incorporated by reference.

In addition, other sequence modifications can be made to the nucleic acid molecules of the invention. For example, additional sequence modifications that are known to enhance gene expression in a cellular host. These include elimination of sequences encoding spurious polyadenylation signals, exon/intron splice site signals, transposon-like repeats, and other such well-characterized sequences that may be deleterious to gene expression. The G-C content of the sequence may also be adjusted to levels average for a target cellular host, as calculated by reference to known genes expressed in the host cell. In addition, the sequence can be modified to avoid predicted hairpin secondary mRNA structures.

Other nucleic acid sequences may also be used in the preparation of the expression cassettes of the present invention, for example to enhance the expression of the HST encoding nucleic acid molecule sequence. Such nucleic acid sequences include the introns of the maize AdhI, Intron I gene (Callis et al. (1987) Genes and Development 1:1183-1200), and leader sequences, (W-sequences) from the Tobacco Mosaic virus (TMV). Maize Chlorotic Mottle Virus and Alfalfa Mosaic Virus (Gallie et al (1987) Nucleic Acid Res. 15:8693-8711, and Skuzeski et al. (1990) Plant Mol. Biol. 15:65-79, 1990). The first Intron rom the shrunken-1 locus of maize has been shown to increase expression of genes in chimeric gene constructs. U.S. Pat. Nos. 5,424,412 and 5,953,874 disclose the use of specific introns in gene expression constructs, and Gallie et al ((1994) Plan Physiol. 106:929-939) also have shown that introns are useful for regulating gene expression on a tissue specific basis. Plant cells transformed with such modified expression cassettes or vectors, then, may exhibit overexpression or constitutive expression of a nucleotide molecule of the invention.

Expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86.6126-6130): potyvirus leaders, for example, TEV leader (Tobacco Etch Virus) (Gallie et al. (1995) Gene 165(2):233-238), MDMV leader (Maize Dwarf Mosaic virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325: 622-625); tobacco mosaic virus leader (TMV) (Gallie et al. (1989) in Molecular Biology of RNA, ed. Cech (Liss. New York). pp. 237-2%); and maize chlorotic mottle virus leader (MCMV) (Lommel et at. (1991) Virology 81:382-385) See also, Della-Cloppa et al. (1987) Plant Physiol 84:965-968.

In preparing the expression cassettes and expression vectors described herein, the various nucleic acid molecules may be manipulated, so as to provide for the nucleic acid molecules in the proper orientation and, as appropriate, in the proper reading frame. Toward this end, adapters or linkers may be employed to join the nucleic acid molecules or other manipulations may be involved to provide for convenient restriction sites, removal of superfluous nucleic acid molecules, removal of restriction sites, or the like. For this purpose, in vitro mutagenesis, primer repair, restriction, annealing, resubstitutions, e.g., transitions and transversions, may be involved.

The expression cassettes of the present invention can also include nucleic acid sequences capable of directing the expression of the HST sequence to the chloroplast. Such nucleic acid sequences include chloroplast targeting sequences that encodes a chloroplast transit peptide to direct the gene product of interest to plant cell chloroplasts. Such transit peptides are known in the art. With respect to chloroplast-targeting sequences, “operably linked” means that the nucleic acid sequence encoding a transit peptide (i.e., the chloroplast-targeting sequence) is linked to the HST nucleic acid molecule of the invention such that the two sequences are contiguous and in the same reading frame. See, for example, Von Heijne et al. (1991) Plant Mol. Biol. Rep. 9:104-126; Clark et al. (1989) J Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1988) Science 233 AI S-4SI. While the HST enzymes of the invention may include a native chloroplast transit peptide, any chloroplast transit peptide known in the art can be fused to the amino acid sequence of a mature HST enzyme of the invention by operably linking a choloroplast-targeting sequence to the 5′-end of a nucleotide sequence encoding a mature HST enzyme of the invention.

Chloroplast targeting sequences are known in the art and include the chloroplast small subunit of ribulose-1,5-bisphosphate carboxylase (Rubisco) (de Castro Silva Filho et al. (1996) Plant Mol. Biol. 30: 769-780; Schnell et al. (1991) J Biol. Chem. 266(5): 3335-3342): 5-(enolpyruvyl)shikimate-3-phosphate synthase (EPSPS) (Archer et al. (1990) J. Bioenerg. Biomemb. 22(6): 789-810); tryptophan synthase (Zhao et al. 1995) J Biol. Chem. 270(1 I): 6081-6087): plastocyanin (Lawrence et al. (1997) J Biol. Chem. 272(33): 20357-20363); chorismate synthase (Schmidt et al. (1993) J Biol. Chem. 268(36): 27447-27457); and the light harvesting chlorophyll a/b binding protein (LHBP) (Lamppa et al. (1988) J Biol. Chem. 263:14996-14999) See also Von Heijne et al. (1991) Plant Mol. Biol. Rep. 9: 104-126; Clark et al. (1989) J Biol. Chem. 264: 17544-17550; Della-Cloppa et al. (1987) Plant Physiol. 84: 965-988; Romer et al. 1993) Biochem. Biophys. Res. Commun. 196: 1414-1421; and Shah et al. (1986) Science 233: 478-481.

The expression cassettes and vectors may be prepared to direct the expression of the HST encoding nucleic acid molecule from the plant cell chloroplast. Methods for transformation of chloroplasts are known in the art. See, for example, Svab et al. (1990) Proc Natl. Acad. Sci. USA 87: 8526-8530: Svab and Maliga (1993) Proc Natl. Acad. Sci. USA 90: 913-917; Svab and Maliga (1993) EMBO J. 12: 601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91: 7301-7305.

The nucleic acids molecules to be targeted to the chloroplast may be optimized for expression in the chloroplast to account for differences in codon usage between the plant nucleus and this organelle. In this manner, the nucleic acids molecules may be synthesized using chloroplast-preferred codons. See, for example, U.S. Pat. No. 5,380,831 herein incorporated by reference.

Expression vectors may include additional features. For example, gRNA promoters to regulate expression of the at least one gRNA, e.g. prOsU3-01, which is the Rice U3 promoter for pol III dependent transcription of non-coding. Vectors may similarly include additional features such as selectable markers, e.g. Phosphomannose Isomerase (PMI), and antibiotic resistance genes that can be used to aid recovery of stably transformed plants.

By “operably linked” or “operably associated” as used herein, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence or nucleic acid molecule that is operably linked to a second nucleotide sequence or nucleic acid molecule, means a situation when the first nucleotide sequence or nucleic acid molecule is placed in a functional relationship with the second nucleotide sequence or nucleic acid molecule. For instance, a promoter is operably associated with a nucleotide sequence or nucleic acid molecule if the promoter effects the transcription or expression of said nucleotide sequence or nucleic acid molecule. Those skilled in the art will appreciate that the control sequences (e.g., promoter) need not be contiguous with the nucleotide sequence or nucleic acid molecule to which it is operably associated, as long as the control sequences function to direct the expression thereof. Thus, for example, intervening untranslated, yet transcribed, sequences can be present between a promoter and a nucleotide sequence or nucleic acid molecule, and the promoter can still be considered “operably linked” to or “operatively associated” with the nucleotide sequence or nucleic acid molecule.

### Herbicide Resistance

The nucleic acids described herein and the HST enzymes encoded thereby may be at least partially resistant to inhibition by an HST-inhibiting herbicide.

At least partially resistant to inhibition by an HST-inhibiting herbicide refers to an HST enzyme that has improved or increased enzymatic activity, relative to the HST activity of a wild-type HST protein, when in the presence of at least one herbicide that is known to interfere with HST activity and at a concentration or level of the herbicide that is to known to inhibit the HST activity of the wild-type HST protein. Partially resistant HST enzymes may have some decrease in enzymatic activity when exposed to an HST-inhibiting herbicide, such as at most a 1%, 2%, 3%, 4%, 5%, 6%, 7%, 8%, 9%, 10%, 15%, 20%, 25%, 30%, 35%, 40%, 45%, 50%, decrease in enzymatic activity.

However, any decreases in activity is less than a decrease in activity relative to the HST activity of a wild-type HST protein, when in the presence of at least one herbicide that is known to interfere with HST activity and at a concentration or level of the herbicide that is to known to inhibit the HST activity of the wild-type HST protein. A decrease in activity seen for a partially resistant HST enzyme may be a decrease in activity that does not have a negative effect on the growth, propagation or development of a plant comprising a partially resistant HST enzyme. Furthermore, the HST activity of such a partially resistant HST protein may be referred to herein as “herbicide-tolerant” or “herbicide-resistant” HST enzyme. Plants which are at least partially “resistant” to the herbicide exhibit few, if any, necrotic, lytic, chlorotic or other lesions when subjected to the herbicide at concentrations and rates which are typically employed by the agricultural community to kill unwanted vegetation in the vicinity of the plant such as a field.

The HST enzymes may be compared to a control or wild-type HST enzyme. The term “wild-type” is used to refer to a nucleic acid molecule or protein that can be found in nature as distinct from being artificially produced or mutated by man. A control or wild-type HST enzyme may be an HST enzyme that does not include the mutations of the invention as described herein. The use of the term “wild-type” or “control” is not intended to necessarily imply that a plant, plant tissue, plant cell, or other host cell lacks recombinant DNA in its genome, and/or does not possess herbicide resistant characteristics that are different from those disclosed herein.

Control HST enzymes may include other mutations or modifications that do not affect resistance to HST inhibiting herbicides. For example, a control HST enzyme may include mutations or modifications to improve or alter expression, translation or targeting of the control HST enzyme to specific tissues, organs or cells.

A wild-type or control HST enzyme may be an HST enzyme encoded by SEQ ID NO: 14. A wild-type or control HST enzyme may be an HST enzyme encoded by SEQ ID NO: 16. A wild-type or control HST enzyme may be an HST enzyme encoded by SEQ ID NO: 18. A wild-type or control HST enzyme may be an HST enzyme encoded by SEQ ID NO: 20. A wild-type or control HST enzyme may be an HST enzyme encoded by SEQ ID NO: 21.

### Examples of HST-Inhibiting Compounds/Herbicides Include:

B1 Compounds disclosed in WO03/016286, which is expressly incorporated herein by reference, including 6-chloro-3-(2-cyclopropyl-6-methylphenoxy)pyridazin-4-yl morpholine-4-carboxylate (cyclopyrimorate) (B1a).

B2 Compounds disclosed in WO2015/168010 and/or WO2020/068057 which are expressly incorporated herein by reference, including but not limited to 6-chloro-5-hydroxy-2-methyl-4-(2-methyl-1-naphthyl)pyridazin-3-one (B2a), 6-chloro-4-(2,7-dimethyl-1-naphthyl)-5-hydroxy-2-methyl-pyridazin-3-one (B2b), 6-chloro-4-(2-methyl-7-chloro-1-naphthyl)-5-hydroxy-2-methyl-pyridazin-3-one (B2c).

B3 Compounds disclosed in WO2016/008816, which is expressly incorporated herein by reference, including but not limited to 4-(2-benzyloxy-3-chloro-6-fluoro-phenyl)-5-hydroxy-2,6-dimethyl-pyridazin-3-one (B3a), 4-(2-benzyloxy-3-chloro-6-fluoro-phenyl)-5-hydroxy-6-methyl-2-prop-2-ynyl-pyridazin-3-one (B3b), 4-(2-benzyloxy-3,6-dichloro-phenyl)-2-cyclopropyl-5-hydroxy-6-methyl-pyridazin-3-one (B3c), 4-[3,6-dichloro-2-[(3,4-dichlorophenyl)methoxy]phenyl]-5-hydroxy-2,6-dimethyl-pyridazin-3-one (B3d), 4-(2-benzyloxy-3,6-dichloro-phenyl)-6-cyclopropyl-5-hydroxy-2-methyl-pyridazin-3-one, 4-(2-benzyloxy-3-chloro-6-fluoro-phenyl)-2-cyclopropyl-5-hydroxy-6-methyl-pyridazin-3-one (B3e).

B4 Compounds disclosed in WO2015/174072, which is expressly incorporated herein by reference, including but not limited to 4-[3,6-dichloro-2-[(2-chlorothiazol-5-yl)methoxy]phenyl]-5-hydroxy-2,6-dimethyl-pyridazin-3-one (B4a), 4-[3,6-dichloro-2-(3-pyridylmethoxy)phenyl]-5-hydroxy-2,6-dimethyl-pyridazin-3-one (B4b), 4-[3-chloro-6-fluoro-2-(thiazol-2-ylmethoxy)phenyl]-5-hydroxy-2,6-dimethyl-pyridazin-3-one (B4c), 4-[3,6-dichloro-2-(thiazol-5-ylmethoxy)phenyl]-5-hydroxy-2,6-dimethyl-pyridazin-3-one (B4d), 4-[3-chloro-2-[(2-chlorothiazol-5-yl)methoxy]-6-fluoro-phenyl]-5-hydroxy-6-methyl-2-prop-2-ynyl-pyridazin-3-one (B4e), 4-[3,6-dichloro-2-[(5-methylpyrazin-2-yl)methoxy]phenyl]-5-hydroxy-2,6-dimethyl-pyridazin-3-one (B4f), 4-[3,6-dichloro-2-[(2-chloro-4-pyridyl)methoxy]phenyl]-5-hydroxy-2,6-dimethyl-pyridazin-3-one (B4g), 4-[3-chloro-6-fluoro-2-[(2-methylthiazol-4-yl)methoxy]phenyl]-5-hydroxy-2,6-dimethyl-pyridazin-3-one (B4h).

B5 Compounds disclosed in WO2019/137851, which is expressly incorporated herein by reference, including but not limited to 4-[3-chloro-6-fluoro-2-[2-(2-fluoro-4-pyridyl)ethyl]phenyl]-5-hydroxy-2,6-dimethyl-pyridazin-3-one (B5a), 4-[3-chloro-6-fluoro-2-[2-(1-methylpyrazol-4-yl)ethyl]phenyl]-5-hydroxy-2,6-dimethyl-pyridazin-3-one (B5b), 4-[3-chloro-6-fluoro-2-(2-phenylethyl)phenyl]-5-hydroxy-2,6-dimethyl-pyridazin-3-one (B5c), 4-[3-chloro-6-fluoro-2-[2-[6-(trifluoromethyl)-3-pyridyl]ethyl]phenyl]-5-hydroxy-2,6-dimethyl-pyridazin-3-one (B5d), 4-[3-chloro-2-[2-(2-chloro-4-pyridyl)ethyl]-6-fluoro-phenyl]-5-hydroxy-2,6-dimethyl-pyridazin-3-one (B5e), 4-[3-chloro-6-fluoro-2-[2-(4-fluorophenyl)ethynyl]phenyl]-5-hydroxy-2,6-dimethyl-pyridazin-3-one (B5f), [5-[3-chloro-6-fluoro-2-[2-(4-fluorophenyl)ethynyl]phenyl]-1,3-dimethyl-6-oxo-pyridazin-4-yl]2-methylpropanoate (B5g), [5-[3-chloro-6-fluoro-2-[2-(p-tolyl)ethyl]phenyl]-1,3-dimethyl-6-oxo-pyridazin-4-yl]methyl carbonate (B5h), [5-[3-chloro-6-fluoro-2-[2-(p-tolyl)ethyl]phenyl]-1,3-dimethyl-6-oxo-pyridazin-4-yl]acetate (B5i), [5-[3-chloro-6-fluoro-2-[2-(p-tolyl)ethyl]phenyl]-1,3-dimethyl-6-oxo-pyridazin-4-yl]benzoate (B5j), [5-[3-chloro-6-fluoro-2-[2-(p-tolyl)ethyl]phenyl]-1,3-dimethyl-6-oxo-pyridazin-4-yl]morpholine-4-carboxylate (B5k), 4-[3-chloro-6-fluoro-2-[(E)-2-thiazol-5-ylvinyl]phenyl]-5-hydroxy-2,6-dimethyl-pyridazin-3-one (B5l), 4-[3-chloro-6-fluoro-2-[2-[2-(trifluoromethyl)phenyl]ethyl]phenyl]-5-hydroxy-2,6-dimethyl-pyridazin-3-one (B5m), 4-[2-fluoro-6-[2-(2-fluoro-4-pyridyl)ethyl]phenyl]-5-hydroxy-2,6-dimethyl-pyridazin-3-one (B5n), [5-[3-chloro-6-fluoro-2-[2-(2-methyltriazol-4-yl)ethyl]phenyl]-1,3-dimethyl-6-oxo-pyridazin-4-yl]2-methylpropanoate (B5o), [5-[3-chloro-6-fluoro-2-[2-(4-fluorophenyl)ethyl]phenyl]-1,3-dimethyl-6-oxo-pyridazin-4-yl]2-methylpropanoate (B5p), 4-[3-chloro-2-[2-(4-cyclopropylphenyl)ethyl]-6-fluoro-phenyl]-5-hydroxy-2,6-dimethyl-pyridazin-3-one (B5q), 4-[3-chloro-6-fluoro-2-(2-pyrimidin-5-ylethyl)phenyl]-5-hydroxy-2,6-dimethyl-pyridazin-3-one (B5r), 4-[3-chloro-6-fluoro-2-[2-(3-thienyl)ethyl]phenyl]-5-hydroxy-2,6-dimethyl-pyridazin-3-one (B5s), 4-[3-chloro-6-fluoro-2-[2-(5-methyl-1,3,4-oxadiazol-2-yl)ethyl]phenyl]-5-hydroxy-2,6-dimethyl-pyridazin-3-one (B5t), [5-[3-chloro-6-fluoro-2-[2-[4-(trifluoromethyl)phenyl]ethyl]phenyl]-1,3-dimethyl-6-oxo-pyridazin-4-yl]2-methylpropanoate (B5u).

B6 Compounds disclosed in WO2020/204112, which is expressly incorporated herein by reference, including but not limited to 4-[5-fluoro-2-(4-fluorophenyl)benzothiophen-3-yl]-5-hydroxy-2,6-dimethyl-pyridazin-3-one (B6a), 5-hydroxy-4-[2-[4-(methoxymethyl)phenyl]benzothiophen-3-yl]-2,6-dimethyl-pyridazin-3-one (B6b), 4-[5-fluoro-3-(5-hydroxy-2,6-dimethyl-3-oxo-pyridazin-4-yl)benzothiophen-2-yl]benzonitrile (B6c), 4-[5-fluoro-2-(4-methylsulfonylphenyl)benzothiophen-3-yl]-5-hydroxy-2,6-dimethyl-pyridazin-3-one (B6c), 4-[2-(2-ethoxy-4-fluoro-phenyl)-6-methyl-benzothiophen-3-yl]-5-hydroxy-2,6-dimethyl-pyridazin-3-one (B6e), [5-[2-(2-ethoxy-4-fluoro-phenyl)-6-fluoro-benzothiophen-3-yl]-1,3-dimethyl-6-oxo-pyridazin-4-yl]hexanoate (B6f), 4-[6-fluoro-2-(4-fluorophenyl)benzothiophen-3-yl]-5-hydroxy-2,6-dimethyl-pyridazin-3-one (B6g), 4-[2-(2-ethoxy-4-fluoro-phenyl)-6-fluoro-benzothiophen-3-yl]-5-hydroxy-2,6-dimethyl-pyridazin-3-one (B6h).

B7 Compounds disclosed in WO2021/009334, which is expressly incorporated herein by reference, including but not limited to 4-[3-chloro-6-fluoro-2-[2-[4-(methylsulfanylmethyl)phenyl]ethyl]phenyl]-5-hydroxy-2,6-dimethyl-pyridazin-3-one (B7a), 4-[3-chloro-6-fluoro-2-[2-(4-methylsulfonylphenyl)ethyl]phenyl]-5-hydroxy-2,6-dimethyl-pyridazin-3-one (B7b), 4-[2-[6-chloro-3-fluoro-2-(5-hydroxy-2,6-dimethyl-3-oxo-pyridazin-4-yl)phenyl]ethyl]-N-ethyl-2-fluoro-N-methyl-benzamide (B7c), [5-[3-chloro-2-[2-[4-[ethyl(methyl)carbamoyl]-3-fluoro-phenyl]ethyl]-6-fluoro-phenyl]-1,3-dimethyl-6-oxo-pyridazin-4-yl]2-methylpropanoate (B7d), 4-[3-cyclopropyl-6-fluoro-2-[2-(2-fluoro-4-pyridyl)ethyl]phenyl]-5-hydroxy-2,6-dimethyl-pyridazin-3-one (B7e), 4-[3-chloro-6-fluoro-2-[2-[4-(1,2,4-triazol-1-yl)phenyl]ethyl]phenyl]-5-hydroxy-2,6-dimethyl-pyridazin-3-one (B7f), 4-[2-[6-chloro-3-fluoro-2-(5-hydroxy-2,6-dimethyl-3-oxo-pyridazin-4-yl)phenyl]ethyl]-N,N-dimethyl-benzenesulfonamide (B7g), 4-[3-chloro-6-fluoro-2-[2-[4-(2-methylthiazol-4-yl)phenyl]ethyl]phenyl]-5-hydroxy-2,6-dimethyl-pyridazin-3-one (B7h), 4-[3-chloro-6-fluoro-2-[2-(4-oxazol-2-ylphenyl)ethyl]phenyl]-5-hydroxy-2,6-dimethyl-pyridazin-3-one (B7i), 4-[3-chloro-6-fluoro-2-[2-(4-pyrazol-1-ylphenyl)ethyl]phenyl]-5-hydroxy-2,6-dimethyl-pyridazin-3-one (B7j), 4-[3-chloro-2-[2-[4-(3,5-dimethylpyrazol-1-yl)phenyl]ethyl]-6-fluoro-phenyl]-5-hydroxy-2,6-dimethyl-pyridazin-3-one (B7k), 4-[3-chloro-6-fluoro-2-[2-[4-(3-methylpyrazol-1-yl)phenyl]ethyl]phenyl]-5-hydroxy-2,6-dimethyl-pyridazin-3-one (B7l), 4-[3-chloro-6-fluoro-2-[2-[4-(5-methyltetrazol-1-yl)phenyl]ethyl]phenyl]-5-hydroxy-2,6-dimethyl-pyridazin-3-one (B7m), 4-[3-chloro-6-fluoro-2-[2-[4-(1-methylpyrazol-3-yl)phenyl]ethyl]phenyl]-5-hydroxy-2,6-dimethyl-pyridazin-3-one (B7n), 3-[(E)-2-[6-chloro-3-fluoro-2-(5-hydroxy-2,6-dimethyl-3-oxo-pyridazin-4-yl)phenyl]vinyl]benzonitrile (B7o), 4-[3-chloro-6-fluoro-2-[(E)-2-(4-hydroxyphenyl)vinyl]phenyl]-5-hydroxy-2,6-dimethyl-pyridazin-3-one (B7p), [5-[3-chloro-2-[(E)-2-(2-cyanophenyl)vinyl]-6-fluoro-phenyl]-1,3-dimethyl-6-oxo-pyridazin-4-yl]2-methylpropanoate (B7q), [5-[2-[2-(4-tert-butoxyphenyl)ethyl]-3-chloro-6-fluoro-phenyl]-1,3-dimethyl-6-oxo-pyridazin-4-yl]2-methylpropanoate (B7r).

B8 Compounds disclosed in WO2021/009335, which is expressly incorporated herein by reference, including but not limited to 4-[3-chloro-6-fluoro-2-[2-(2-methyl-1,3-benzoxazol-6-yl)ethyl]phenyl]-5-hydroxy-2,6-dimethyl-pyridazin-3-one (B8a), 4-[3-chloro-6-fluoro-2-[2-(3-methylbenzotriazol-5-yl)ethyl]phenyl]-5-hydroxy-2,6-dimethyl-pyridazin-3-one (B8b), [5-[3-chloro-2-[(E)-2-(2,2-difluoro-1,3-benzodioxol-5-yl)vinyl]-6-fluoro-phenyl]-1,3-dimethyl-6-oxo-pyridazin-4-yl]2-methylpropanoate (B8c), 4-[3-chloro-6-fluoro-2-[2-(2-methyl-1,3-benzothiazol-5-yl)ethyl]phenyl]-5-hydroxy-2,6-dimethyl-pyridazin-3-one (B8d), 4-[3-chloro-6-fluoro-2-[2-(1-methylindol-5-yl)ethyl]phenyl]-5-hydroxy-2,6-dimethyl-pyridazin-3-one (B8e), 4-[3-chloro-6-fluoro-2-[2-(1-methylindol-6-yl)ethyl]phenyl]-5-hydroxy-2,6-dimethyl-pyridazin-3-one (B8f), 4-[3-chloro-6-fluoro-2-[2-(3-methyl-1,2-benzoxazol-6-yl)ethyl]phenyl]-5-hydroxy-2,6-dimethyl-pyridazin-3-one (B8g), 5-[2-[6-chloro-3-fluoro-2-(5-hydroxy-2,6-dimethyl-3-oxo-pyridazin-4-yl)phenyl]ethyl]indolin-2-one (B8h), 4-[3-chloro-6-fluoro-2-[2-(1-methylindazol-6-yl)ethyl]phenyl]-5-hydroxy-2,6-dimethyl-pyridazin-3-one (B8i), 4-[3-chloro-6-fluoro-2-[2-(1-methylindazol-5-yl)ethyl]phenyl]-5-hydroxy-2,6-dimethyl-pyridazin-3-one (B8j), 4-[3-chloro-6-fluoro-2-[2-(1-methylindazol-3-yl)ethyl]phenyl]-5-hydroxy-2,6-dimethyl-pyridazin-3-one (B8k), 4-[3-chloro-6-fluoro-2-[2-(1-methylpyrrolo[2,3-b]pyridin-5-yl)ethyl]phenyl]-5-hydroxy-2,6-dimethyl-pyridazin-3-one (B8l), 5-[2-[6-chloro-3-fluoro-2-(5-hydroxy-2,6-dimethyl-3-oxo-pyridazin-4-yl)phenyl]ethyl]-3H-1,3-benzoxazol-2-one (B8m), 4-[2-[2-(1,3-benzoxazol-2-yl)ethyl]-3-chloro-6-fluoro-phenyl]-5-hydroxy-2,6-dimethyl-pyridazin-3-one (B8n), 4-[2-[2-(1,3-benzothiazol-6-yl)ethyl]-3-chloro-6-fluoro-phenyl]-5-hydroxy-2,6-dimethyl-pyridazin-3-one (B8o), 4-[3-chloro-6-fluoro-2-[(E)-2-(2-methyl-6-quinolyl)vinyl]phenyl]-5-hydroxy-2,6-dimethyl-pyridazin-3-one (B8p), 4-[2-[2-(benzothiophen-6-yl)ethyl]-3-chloro-6-fluoro-phenyl]-5-hydroxy-2,6-dimethyl-pyridazin-3-one (B8q).

The HST-inhibiting herbicide may be any combination of HST-inhibiting herbicides. For example, 1, 2, 3, 4, 5 or more HST-inhibiting herbicides as described herein.

In particular the HST-inhibiting herbicide may be selected from one or more of:
4-[3-chloro-6-fluoro-2-[2-(2-fluoro-4-pyridyl)ethyl]phenyl]-5-hydroxy-2,6-dimethyl-pyridazin-3-one (which may also be referred to as compound B5a);
4-[3-chloro-6-fluoro-2-[2-(1-methylpyrazol-4-yl)ethyl]phenyl]-5-hydroxy-2,6-dimethyl-pyridazin-3-one (which may also be referred to as compound B5b);
[5-[3-chloro-6-fluoro-2-[2-[4-(trifluoromethyl)phenyl]ethyl]phenyl]-1,3-dimethyl-6-oxo-pyridazin-4-yl]2-methylpropanoate (which may also be referred to as compound B5u); and/or
4-[3-chloro-6-fluoro-2-[2-(2-methyl-1,3-benzoxazol-6-yl)ethyl]phenyl]-5-hydroxy-2,6-dimethyl-pyridazin-3-one (which may also be referred to as compound B8a).

### Plants

The nucleic acid molecules, HST enzymes and/or vectors of the invention may be provided in or introduced into a plant or part thereof. Thus, also provided herein there are plants, progeny thereof, and parts thereof that include a nucleic acid molecule, HST enzyme or an expression vector as described herein.

The present invention may be for use with any plant species and the progeny thereof, including, but not limited to, monocots and dicots. Examples of plant species of interest include, but are not limited to, corn or maize (Zea mays), Brassica sp. (e.g., B. napus, B. rapa, B. juncea), including those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum, T. Turgidum ssp. durum), soybean (Glycine max), tobacco (Nicotiana tabacum), potato (Solarium tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Primus amygdalus), sugar beets (Beta vulgaris), sugarcane (Saccharum spp.), oats (Avena sativa), barley (Hordeum vulgare), vegetables, ornamentals, and conifers. Preferably, plants of the present invention are crop plants (for example, sunflower, Brassica sp., cotton, sugar, beet, soybean, peanut, alfalfa, safflower, tobacco, corn, rice, wheat, rye, barley triticale, sorghum, millet, etc.).

As used herein, the terms “progeny” and “progeny plant” refer to a plant generated from a vegetative or sexual reproduction from one or more parent plants. A progeny plant may be obtained by cloning or selfing a single parent plant, or by crossing two parental plants.

As used herein unless clearly indicated otherwise, the term “plant” is intended to mean a plant at any developmental stage, as well as any part or parts of a plant that may be attached to or separate from a whole intact plant. Such parts of a plant include, but are not limited to, organs, tissues, and cells of a plant including, plant calli, plant clumps, plant protoplasts and plant cell tissue cultures from which plants can be regenerated. Examples of particular plant parts include a stem, a leaf, a root, an inflorescence, a flower, a floret, a fruit, a pedicle, a peduncle, a stamen, an anther, a stigma, a style, an ovary, a petal, a sepal, a carpel, a root tip, a root cap, a root hair, a leaf hair, a seed hair, a pollen grain, a microspore, an embryos, an ovule, a cotyledon, a hypocotyl, an epicotyl, xylem, phloem, parenchyma, endosperm, a companion cell, a guard cell, and any other known organs, tissues, and cells of a plant. Furthermore, it is recognized that a seed is a plant part.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant. A “plant organ” is a distinct and visibly structured and differentiated part of a plant such as a root, stem, leaf, flower bud, or embryo.

The plants, progeny thereof or parts thereof of the invention express at least one of the nucleic acid molecules, HST enzymes or expression vectors of the invention. Expression of the nucleic acid molecules, HST enzymes or expression vectors of the invention provide a plant that is at least partially resistant to HST-inhibiting herbicides, such as those herbicides described herein. For example, the plants, progeny thereof or parts thereof of the invention may have increased resistance to an HST inhibiting herbicide. The increase in resistance may be determined by comparison to a wild-type or control plant as described herein. For example, a plant that does not include or express nucleic acid molecules, HST enzymes or expression vectors of the invention.

A plant having increased resistance to an HST-inhibiting herbicide may be referred to as an “herbicide-tolerant” or “herbicide-resistant” plant. Such plants are tolerant or at least partially resistant to at least one HST-inhibiting herbicide at a level that would normally kill, or inhibit the growth of, a normal, control or wild-type plant lacking nucleic acid molecules, mutated HST enzymes or expression vectors of the invention.

For example, plants of the invention may have at least a 2-fold increase in resistance to HST inhibiting herbicides, such as the HST inhibiting herbicides described herein. For example, plants of the invention may have at least a 2-fold, 3-fold, 4-fold, 5-fold 6-fold, 7-fold, 8-fold, 9-fold, 10-fold increase in resistance.

Resistance to HST inhibiting herbicides may be determined by any known methods for comparing the growth, damage or other properties of two plants after application of an HST-inhibiting herbicide to a plant. For example, the resistance of a plant of the invention may be determined by comparing the percentage of damaged caused to the plant in comparison to a wild-type or control plant after application of an HST inhibiting herbicide.

Without being bound by theory, increased resistance may be provided by an increase in the activity of the mutated HST enzymes of the invention in comparison to wild-type or control HST enzymes. The mutated HST enzymes of the invention may also not be less susceptible to HST inhibiting herbicides, for example due to changes in the structure of the enzyme leading to reduced binding of HST inhibiting herbicides.

Thus provided herein are methods of conferring increased resistance to HST inhibiting herbicides to a plant or part thereof by expressing a HST enzyme of the invention. The HST enzyme may be introduced into a plant or part thereof by modifying the plant to contain a nucleic acid sequence or expression vector of the invention. Thus, plants of the invention may be referred to as modified plants.

The invention also includes methods for modifying plants or parts thereof to express the HST enzymes of the invention. Methods of modifying plants may include introducing a nucleic acid molecule according of the invention into a plant or part thereof and expression the nucleic acid molecule to produce a HST enzyme of the invention in the plant or part thereof. In addition to or alternatively a plant may be modified by in situ editing of the plants endogenous genetic material in order to provide a gene that expresses a HST enzyme of the invention.

For example, a plant, a plant part, plant cell or protoplast may be transformed with a nucleic acid or expression vector of the invention. In addition to or alternatively a plant may be transformed with one or more nucleic acid molecules encoding a gene editing system for modifying an endogenous nucleic acid sequence of the plant encoding an HST enzyme at one or more positions to produce a nucleic acid molecule according the invention. Thus providing a plant or part thereof that expresses a HST enzyme of the invention.

“Transformation” refers to a process of introducing an exogenous nucleic acid molecule (for example, a recombinant polynucleotide) into a cell or protoplast and that exogenous nucleic acid molecule is incorporated into a host cell genome or an organelle genome (for example, chloroplast or mitochondria) or is capable of autonomous replication. “Transformed” or “transgenic” refers to a cell, tissue, organ, or organism into which a foreign nucleic acid, such as an expression vector or recombinant nucleic acid molecule has been introduced. The nucleic acid molecule can be stably integrated into the genome of the host or the nucleic acid molecule can also be present as an extrachromosomal molecule. Such an extrachromosomal molecule can be auto-replicating. The nucleic acid molecule can also be introduced into the genome of the chloroplast or the mitochondria of a plant cell.

Methods of transformation of plant cells or tissues include, but are not limited to Agrobacterium mediated transformation method and the Biolistics or particle-gun mediated transformation method. Suitable plant transformation vectors for the purpose of Agrobacterium mediated transformation include-those elements derived from a tumor inducing (Ti) plasmid of Agrobacterium tumefaciens, for example, right border (RB) regions and left border (LB) regions, and others disclosed by Herrera-Estrela et al., Nature 303:209 (1983) Bevan, Nucleic Acids Res. 12:8711-8721 (1984): Klee et al., Bio-Technology 3(7):637-642 (1985). In addition to plant transformation vectors derived from the Ti or root-inducing (Ri) plasmids of Agrobacterium, alternative methods can be used to insert the nucleic acid molecules of this invention into plant cells. Such methods may involve, but are not limited to, for example, the use of liposomes, electroporation, chemicals that increase free DNA uptake, free DNA delivery via microprojectile bombardment, and transformation using viruses or pollen.

A “transgenic” or “transformed” cell or plant also includes progeny of the cell or plant and progeny produced from a breeding program employing such a “transgenic” plant as a parent in a cross and exhibiting an altered phenotype resulting from the presence of the foreign nucleic acid molecule.

The transgenic plants may be homozygous for the nucleic acid molecule encoding a HST enzyme described herein (i.e. those that contain two added genes encoding a HST enzyme at the same position on each chromosome of the chromosome pair). Homozygous transgenic plants may be obtained by crossing (self-pollinating) independent transgenic plant isolates containing a single added gene, germinating some of the resulting seeds, and transforming the resulting plant with the target gene.

An endogenous HST encoding nucleic acid may edited in situ by way of gene editing techniques in order to provide a HST enzyme that is at least partially resistant to a HST-inhibiting herbicides such as those described herein and as such a modified plant as described herein. Such genome editing and/or mutagenesis technologies are well known in the art. As well, introduction may be accomplished by any manner known in the art, including: introgression, transgenic, or site-directed nucleases (SDN). Particularly, the modification to the nucleic acid sequence is introduced by way of site-directed nuclease (SDN). More particularly, the SDN is selected from: meganuclease, zinc finger, transcription activator-like effector nucleases system (TALEN) or Clustered Regularly Interspaced Short Palindromic Repeats system (CRISPR) system. SDN is also referred to as “genome editing”, or genome editing with engineered nucleases (GEEN). This is a type of genetic engineering in which DNA is inserted, deleted or replaced in the genome of an organism using engineered nucleases that create site-specific double-strand breaks (DSBs) at desired locations in the genome. The induced double-strand breaks are repaired through nonhomologous end-joining (NHEJ) or homologous recombination (HR), resulting in targeted mutations (‘edits’). Particularly SDN may comprises techniques such as: Meganucleases, Zinc finger nucleases (ZFNs), Transcription Activator-Like Effector-based Nucleases (TALEN) (Feng et al. 2013 Cell Res. 23; 1229-1232, Sander & Joung Nat. Biotechnol. 32, 347-355 2014), and the Clustered Regularly Interspaced Short Palindromic Repeats (CRISPR-Cas) system. Gene editing may also be achieved by SDN-2. SDN-2 is similar to SDN, but also provides a small nucleotide template complementary to the area of the break. The template contains one or more sequences modifications to the genomic DNA which are incorporated to create the mutation to the target gene. Preferably, the gene editing system may include a CRISPR-Cas system.

As used herein, the term “guide RNA” or “gRNA” generally refers to an RNA molecule (or a group of RNA molecules collectively) that can bind to a CRISPR system effector, such as a Cas or a Cpf 1 protein, and aid in targeting the Cas or Cpf1 protein to a specific location within a target polynucleotide (e.g., a DNA). A guide RNA of the invention can be an engineered, single RNA molecule (sgRNA), where for example the sgRNA comprises a crRNA segment and optionally a tracrRNA segment. A guide RNA of the invention can also be a dual-guide system, where the crRNA and tracrRNA molecules are physically distinct molecules which then interact to form a duplex for recruitment of a CRISPR system effector, such as Cas9, and for targeting of that protein to the target polynucleotide.

As used herein, the term “crRNA” or “crRNA segment” refers to an RNA molecule or to a portion of an RNA molecule that includes a polynucleotide targeting guide sequence, a stem sequence involved in protein-binding, and, optionally, a 3′-overhang sequence. The polynucleotide targeting guide sequence is a nucleic acid sequence that is complementary to a sequence in a target DNA (for example a gene encoding an HST enzyme). This polynucleotide targeting guide sequence is also referred to as the “protospacer”. In other words, the polynucleotide targeting guide sequence of a crRNA molecule interacts with a target DNA in a sequence-specific manner via hybridization (i.e., base pairing). As such, the nucleotide sequence of the polynucleotide targeting guide sequence of the crRNA molecule may vary and determines the location within the target DNA that the guide RNA and the target DNA will interact.

The polynucleotide targeting guide sequence of a crRNA molecule can be modified (e.g., by genetic engineering) to hybridize to any desired sequence within a target DNA. The polynucleotide targeting guide sequence of a crRNA molecule of the invention can have a length from about 12 nucleotides to about 100 nucleotides. For example, the polynucleotide targeting guide sequence of a crRNA can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the polynucleotide targeting guide sequence of a crRNA can have a length of from about 17 nt to about 27 nts.

For example, the polynucleotide targeting guide sequence of a crRNA can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. The nucleotide sequence of the polynucleotide targeting guide sequence of a crRNA can have a length at least about 12 nt. In some embodiments, the polynucleotide targeting guide sequence of a crRNA is 20 nucleotides in length. In some embodiments, the polynucleotide targeting guide sequence of a crRNA is 19 nucleotides in length.

The present invention also provides a guide RNA comprising an engineered crRNA, wherein the crRNA comprises a bait RNA segment capable of hybridizing to a genomic target sequence. This engineered crRNA maybe a physically distinct molecule, as in a dual-guide system.

As used herein, the term “tracrRNA” or “tracrRNA segment” refers to an RNA molecule or portion thereof that includes a protein-binding segment (e.g., the protein-binding segment is capable of interacting with a CRISPR-associated protein, such as a Cas9). The present invention also provides a guide RNA comprising an engineered tracrRNA, wherein the tracrRNA further comprises a bait RNA segment that is capable of binding to a donor DNA molecule. The engineered tracrRNA may be a physically distinct molecule, as in a dual-guide system, or may be a segment of a sgRNA molecule.

The guide RNA, either as a sgRNA or as two or more RNA molecules, does not contain a tracrRNA, as it is known in the art that some CRISPR-associated nucleases, such as Cpf1 (also known as Casl2a), do not require a tracrRNA for its RNA-mediated endonuclease activity (Qi et al., (2013), Cell, 152: 1173-1183; Zetsche et al., (2015), Cell 163: 759-771). Such a guide RNA of the invention may comprise a crRNA with the bait RNA operably linked at the 5′ or 3′ end of the crRNA. Cpf1 also has RNase activity on its cognate pre-crRNA (Fonfara et al., (2016), Nature, doi.org/10.1038/naturel7945).

A guide RNA of the invention may comprise multiple crRNAs which the Cpf1 possesses to mature crRNAs. Each of these crRNAs may be operably linked to a bait RNA. At least one of these crRNAs may be operably linked to a bait RNA. The bait RNA may be specific to a sequence of interest (SOI), or it may be a “universal” bait, which has a corresponding “universal” prey sequence on the donor DNA molecule.

The present invention also provides a nucleic acid molecule comprising a nucleic acid sequence encoding a guide RNA of the invention. The nucleic acid molecule may be a DNA or an RNA molecule. The nucleic acid molecule may be circularized or linear. The nucleic acid molecule may be single stranded, partially double-stranded, or double-stranded. The nucleic acid molecule may be complexed with at least one polypeptide. The polypeptide may have a nucleic acid recognition or nucleic acid binding domain. The polypeptide may be a shuttle for mediating delivery of, for example, a nucleic acid molecule of the invention, a nuclease, and optionally a donor molecule. The polypeptide may be a Feldan Shuttle (U.S. Patent Publication No. 20160298078, herein incorporated by reference). The nucleic acid molecule may comprise an expression cassette capable of driving the expression of the nucleic acid molecule. The nucleic acid molecule may further comprise additional expression cassettes, capable of expressing, for example, a nuclease such as a CRISPR-associated nuclease.

The plants of the present invention include both non-transgenic plants and transgenic plants. By “non-transgenic plant” is intended to mean a plant lacking recombinant DNA in its genome, but containing the mutant nucleic acid molecule in the plant cell genome which has been mutated using mutagenic techniques, such as chemical mutagenesis or by those methods provided herein. Non-transgenic plants may encompass those plants having mutant sequences as a result of natural processes, such as plants including spontaneous HST enzymes that correspond to the HST enzymes of the invention. By “transgenic plant” is intended to mean a plant comprising recombinant DNA in its genome. Such a transgenic plant can be produced by introducing recombinant DNA into the genome of the plant. When such recombinant DNA is incorporated into the genome of the transgenic plant, progeny of the plant can also comprise the recombinant DNA. A progeny plant that comprises at least a portion of the recombinant DNA of at least one progenitor transgenic plant is also a transgenic plant.

The term “spontaneous mutant” refers to mutants or variants that arise from the parent strain without the intentional use of mutagens i.e. they are considered as not genetically modified (non-GMO). Spontaneous mutants in respect of plants may also be known as sports, breaks, or chimeras.

The transformed parts of plants, transformed plant cells or a transformed plant protoplasts as described herein may be regenerated to produce a modified plant as described herein.

When adequate numbers of transformed cells or protoplasts containing the exogenous nucleic acid molecule encoding HST enzymes of the present invention are obtained, the cells can be cultured, then regenerated into whole plants. “Regeneration” refers to the process of growing a plant from a plant cell (for example, plant protoplast or explant). Such regeneration techniques rely on manipulation of certain phytohormones in a tissue culture growth medium, typically relying on a biocide and/or herbicide marker that has been introduced together with the desired nucleotide sequences. Choice of methodology for the regeneration step is not critical. See for See, for example, Ammirato et al., Handbook of Plant Cell Culture—Crop Species. Macmillan Publ. Co. (1984); Shimamoto et al., Nature 338:274-275 (1989); Fromm, UCLA Symposium on Molecular Strategies for Crop Improvement. Apr. 16-22, 1990. Keystone, Colo. 1990); Vasil et al., Bio/Technology 8:429-434 (1990); Vasil et al., Bio/Technology 10:667-674(1992); Hayashimoto, Plant Physiol. 93:857-863 (1990); and Datta et al., Bio-technology 8:736-740 (1990). Such regeneration techniques are described generally in Klee et al., Ann. Rev. Plant Phys. 38:467-486 (1987).

The development or regeneration of transgenic plants containing a nucleic acid molecule that encodes a HST enzyme of the invention is well known in the art. The regenerated plants may be self-pollinated to provide homozygous transgenic plants, as discussed above. Otherwise, pollen obtained from the regenerated plants is crossed to seed-grown plants of agronomically important lines. Conversely, pollen from plants of these important lines is used to pollinate regenerated plants.

The at least partially HST-inhibiting herbicide resistant plants and progeny of such plants described herein (such as transformed, modified or transgenic plants described herein), can be used in methods for preparing at least partially HST-inhibiting herbicide resistant plants, plants having increased tolerance to HST-inhibiting herbicides, and seeds of such plants. Thus, for example, the plants exemplified herein may be used in breeding programs to develop additional at least partially herbicide resistant plants, such as commercial varieties of such plants. In accordance with such methods, a first parent plant may be used in crosses with a second parent plant, where at least one of the first or second parent plants contains at least one nucleic acid molecule encoding a HST herbicide resistant HST enzyme as described herein. One application of the process is in the production of F1 hybrid plants. Another aspect of this process is that the process can be used for the development of novel parent, dihaploid or inbred lines. For example, a plant line as described herein could be crossed to any second plant, and the resulting hybrid progeny each selfed and/or sibbed for about 5 to 7 or more generations, thereby providing a large number of distinct, parent lines. These parent lines could then be crossed with other lines and the resulting hybrid progeny analyzed for beneficial characteristics. In this way, novel lines conferring desirable characteristics could be identified. Various breeding methods may be used in the methods, including haploidy, pedigree breeding, single-seed descent, modified single seed descent, recurrent selection, and backcrossing.

The plants and progeny thereof may display a synergistic effect rather than additive effect of HST-inhibiting herbicide tolerance, whereby the level of herbicide tolerance in the plants and the progeny thereof comprising multiple mutations is greater than the combined herbicide tolerance of plants comprising a single HST protein.

Plant lines containing the nucleic acid molecules of the present invention can be crossed by either natural or mechanical techniques. Mechanical pollination can be effected either by controlling the types of pollen that can be transferred onto the stigma or by pollinating by hand.

Any breeding method may be used in the methods of the present invention. In one example, the herbicide-resistant plants of the present invention may be bred using a haploid method. In such methods, parents having the genetic basis for the desired complement of characteristics are crossed in a simple or complex cross. Crossing (or cross-pollination) refers to the transfer of pollen from one plant to a different plant. Progeny of the cross are grown and microspores (immature pollen grains) are separated and filtered, using techniques known to those skilled in the art [(e.g. Swanson, E B. et al. (1987) Plant Cell Reports, 6: 94.97, “Efficient isolation of microspores and the production of microspore-denved embryos in Brassica napus. L.; and Swanson. E. B., (1990) Microspore culture in Brassica, pp. 159-169 in Methods in Molecular Biology. vol. 6, Plant Cell and Tissue Culture, Humana Press]. These microspores exhibit segregation of genes. The microspores are cultured in the presence of an appropriate AHAS-inhibitor herbicide, such as imazethapyr (e.g. PURSUIT™) or imazamox (e.g. SOLO™, BEYOND™, and RAPTOR™) or a 50/50 mix of imazethapyr and imazamox (e.g. ODYSSEY™) which kills microspores lacking the mutations responsible for resistance to the herbicide. Microspores carrying the genes responsible for resistance to the herbicide survive and produce embryos, which form haploid plants. Their chromosomes are then doubled to produce doubled haploids.

Other breeding methods may also be used in accordance with the present invention. For example, pedigree breeding may be used for the improvement of largely self-pollinating crops such as Brassica and canola. Pedigree breeding starts with the crossing of two genotypes, each of which may have one or more desirable characteristics that is lacking in the other or which complements the other. If the two original parents do not provide all of the desired characteristics, additional parents can be included in the crossing plan. These parents may be crossed in a simple or complex manner to produce a simple or complex F1. An F2 population is produced from the F1 by selfing one or several F1 plants, or by intercrossing two F1's (i.e., sib mating). Selection of the best individuals may begin in the F2 generation, and beginning in the F3 the best families, and the best individuals within the best families are selected. Replicated testing of families can begin in the F4 generation to improve the effectiveness of selection for traits with low heritability. At an advanced stage of inbreeding (i.e., F6 and F7), the best lines or mixtures of phenotypically similar lines may be tested for potential release as new cultivars. However, the pedigree method is more time-consuming than the haploidy method for developing improved At least partially HST-inhibiting herbicide resistant plants, because the plants exhibit segregation for multiple generations, and the recovery of desirable traits is relatively low.

The single seed descent (SSD) procedure may also be used to breed improved varieties. The SSD procedure in the strict sense refers to planting a segregating population, harvesting a sample of one seed per plant, and using the population of single seeds to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the plants originally sampled in the F2 population will be represented by a progeny when generation advance is completed.

In a multiple-seed procedure, canola breeders commonly harvest one or more pods from each plant in a population and thresh them together to form a bulk. Part of the bulk is used to plant the next generation and part is put in reserve. The procedure has been referred to as modified single-seed descent or the pod-bulk technique. The multiple-seed procedure has been used to save labour at harvest. It is considerably faster to thresh pods with a machine than to remove one seed from each by hand for the single-seed procedure. The multiple-seed procedure also makes it possible to plant the same number of seeds of a population each generation of inbreeding. Enough seeds are harvested to make up for those plants that did not germinate or produce seed.

Backcross breeding can be used to transfer a gene or genes for a simply inherited, highly heritable trait from a source variety or line (the donor parent) into another desirable cultivar or inbred line (the recurrent parent). After the initial cross, individuals possessing the phenotype of the donor parent are selected and are repeatedly crossed (backcrossed) to the recurrent parent. When backcrossing is complete, the resulting plant is expected to have the attributes of the recurrent parent and the desirable trait transferred from the donor parent.

Improved varieties may also be developed through recurrent selection. In this method, genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

At least partially HST-inhibiting herbicide resistant plants can be produced by cross-pollinating a first plant with a second plant and allowing the pollen acceptor plant (can be either the first or second plant) to produce seed from this cross pollination. Seeds and progeny plants generated therefrom can have the mutation crossed into the genome of the seed and/or progeny plants. The pollen-acceptor plant can be either the first or second plant. The first plant comprises a first nucleic acid molecule encoding at least one HST mutant enzyme as disclosed herein. The second plant can be any compatible plant and may comprise a second nucleic acid molecule encoding the same or different HST mutant enzyme. The first and second HST enzymes may comprise the same or different amino acid substitution(s) or deletions relative to a wild-type HST enzyme. Seeds or progeny plants arising from the cross which comprise one nucleic acid molecule encoding the HST mutant enzyme or two nucleic acid molecules encoding the two HST mutant enzymes can be selected.

When the first and second plants are homozygous for the first and second nucleic acid molecules, respectively, each of the resulting progeny plants comprises one copy of each of the first and second nucleic acid molecules and the selection step can be omitted. When at least one of the first and second plants is heterozygous, progeny plants comprising both nucleic acid molecules can be selected, for example, by analyzing the DNA of progeny plants to identify progeny plants comprising both the first and second nucleic acid molecules or by testing the progeny plants for increased herbicide tolerance.

Descendent and/or progeny plants may be evaluated for the nucleic acid molecules of the present invention by any method to determine the presence of a mutated HST nucleic acid or enzyme.

Therefore, also provided herein are methods of selecting a plant or part thereof that includes a nucleic acid molecule, expression vector or HST enzyme of the invention by exposing the plant or part thereof to an effective amount of an HST-inhibiting herbicide sufficient to prevent or reduce the growth of a plant that does not include at least nucleic acid molecule, expression vector or HST enzyme of the invention. It may then be determined by the methods described herein whether the plant has been effected (e.g. has reduced growth or reduced damage) by the HST inhibiting herbicide. Plants that are unaffected by the HST-inhibiting herbicide may then be selected.

Methods of determining whether a plant includes the nucleic acid, expression vector or HST enzyme of the invention and/or is effected by an HST-inhibiting herbicide include phenotypic evaluations, genotypic evaluations, or combinations thereof. The progeny plants may be evaluated in subsequent generations for herbicide resistance, and other desirable traits. Resistance to HST-inhibiting herbicides may be evaluated by exposing plants to one or more appropriate HST-inhibiting herbicides and evaluating herbicide injury. Some traits, such as lodging resistance and plant height, may be evaluated through visual inspection of the plants, while earliness of maturity may be evaluated by a visual inspection of seeds within pods (siliques). Other traits, such as oil percentage, protein percentage, and total glucosinolates of seeds may be evaluated using techniques such as Near Infrared Spectroscopy and/or liquid chromatography and/or gas chromatography.

Plants of the present invention can also be identified using any genotypic analysis method. Genotypic evaluation of the plants includes using techniques such as Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Allele-specific PCR (AS-PCR), DNA Amplification Fingerprinting (DAF), Sequence Characterized Amplified Regions (SCARs), Amplified Fragment Length Polymorphisms (AFLPs), Simple Sequence Repeats (SSRs) which are also referred to as “Microsatellites”. Additional compositions and methods for analyzing the genotype of the plants provided herein include those methods disclosed in U.S. Publication No. 2004/0171027, U.S. Publication No. 2005/02080506, and U S Publication No. 2005/0283858, the entireties of which re hereby incorporated by reference.

Evaluation and manipulation (through exposure to one or more appropriate HST-inhibiting herbicides) may occur over several generations. The performance of the new lines may be evaluated using objective criteria in comparison to check varieties. Lines showing the desired combinations of traits are either crossed to another line or self-pollinated to produce seed.

“Sequencing DNA” refers to determining the nucleic acid sequence of a piece of DNA, e.g. of a gene. Standard methods and commercial services are known in the art. Basic methods for DNA sequencing include the Maxam-Gilbert method and the chain termination method. High-throughput techniques have also been developed and are preferably used in the method of the present invention. These high-throughput techniques include, but are not limited to, Massively parallel signature sequencing (MPSS), Polony sequencing, 454 pyrosequencing, Illumina (Solexa) sequencing, Combinatorial probe anchor synthesis (cPAS), SOLiD sequencing, Ion Torrent semiconductor sequencing, DNA nanoball sequencing, Heliscope single molecule sequencing, Single molecule real time (SMRT) sequencing and Nanopore DNA sequencing.

Sequencing may be carried out using primers that are capable of binding to a nucleic acid molecule of the invention. For example, primers that complimentary to at least a portion of a nucleic acid molecule of the invention.

As used herein, the term “primer” refers to an oligonucleotide which is capable of annealing to a nucleic acid target and serving as a point of initiation of DNA synthesis when placed under conditions in which synthesis of a primer extension product is induced (e.g., in the presence of nucleotides and an agent for polymerization such as DNA polymerase and at a suitable temperature and pH). A primer (in some examples an extension primer and in some examples an amplification primer) may be single stranded for maximum efficiency in extension and/or amplification. The primer may be an oligodeoxyribonucleotide. A primer is typically sufficiently long to prime the synthesis of extension and/or amplification products in the presence of the agent for polymerization. The minimum length of the primer can depend on many factors, including, but not limited to temperature and composition (A/T vs. G/C content) of the primer. In the context of amplification primers, these are typically provided as a pair of bi-directional primers consisting of one forward and one reverse primer or provided as a pair of forward primers as commonly used in the art of DNA amplification such as in PCR amplification.

As such, it will be understood that the term “primer,” as used herein, can refer to more than one primer, particularly in the case where there is some ambiguity in the information regarding the terminal sequence(s) of the target region to be amplified. Hence, a “primer” can include a collection of primer oligonucleotides containing sequences representing the possible variations in the sequence or includes nucleotides which allow a typical base pairing. Primers can be prepared by any suitable method known in the art. Methods for preparing oligonucleotides of specific sequence are known in the art, and include, for example, cloning and restriction of appropriate sequences and direct chemical synthesis. Chemical synthesis methods can include, for example, the phospho di- or tri-ester method, the diethylphosphoramidate method and the solid support method disclosed in U.S. Pat. No. 4,458,066.

Primers can be labelled, if desired, by incorporating detectable moieties by for instance spectroscopic, fluorescence, photochemical, biochemical, immunochemical, or chemical moieties. Primers diagnostic (i.e. able to identify or select based on presence of HST encoding nucleic acids and the HST enzymes thereof as described herein) for HST resistance can be created by any known methods. The PCR method is well described in handbooks and known to the skilled person. After amplification by PCR, target polynucleotides can be detected by hybridization with a probe polynucleotide, which forms a stable hybrid with the target sequence under stringent to moderately stringent hybridization and wash conditions. If it is expected that the probes are essentially completely complementary (i.e., about 99% or greater) to the target sequence, stringent conditions can be used.

If some mismatching is expected, for example if variant nucleic acids are expected with the result that the probe will not be completely complementary, the stringency of hybridization can be reduced. In some examples, conditions are chosen to rule out non-specific/adventitious binding. Conditions that affect hybridization, and that select against non-specific binding are known in the art, and are described in, for example, Sambrook & Russell (2001) Molecular Cloning: A Laboratory Manual, Third Edition, Cold Spring Harbor Laboratory Press. Cold Spring Harbor, New York, United Stated of America. Generally, lower salt concentration and higher temperature hybridization and/or washes increase the stringency of hybridization conditions.

Also included herein are seeds that are capable of producing a plant that includes a nucleic acid molecule, HST enzyme or expression vector of the invention.

The term “seed” embraces seeds and plant propagules of all kinds including but not limited to true seeds, seed pieces, suckers, corms, bulbs, fruit, tubers, grains, cuttings, cut shoots and the like.

Seeds may be treated or untreated seeds. For example, the seeds can be treated to improve germination, for example, by priming the seeds, or by disinfection to protect against seed-borne pathogens. In another example, seeds can be coated with any available coating to improve, for example, plantability, seed emergence, and protection against seed-borne pathogens. Seed coating can be any form of seed coating including, but not limited to pelleting, film coating, and encrustments.

The seed may be germinated and used to produce or grow a plant or part thereof of the invention. That is a plant including a nucleic acid molecule, HST enzyme or expression vector of the invention.

Also provided herein is a container including seeds of the invention. A container of seeds may contain any number, weight or volume of seeds. For example, a container can contain at least, or greater than, about 10, 25, 50, 75, 100, 200, 300, 400, 500, 600, 700, 800, 900, 1000, 1500, 2000, 2500, 3000, 3500, 4000, 4500, 5000 or more seeds. Alternatively, the container can contain at least, or greater than, about 1 ounce, 5 ounces, 10, ounces, 1 pound, 2 pounds, 3 pounds, 4 pounds, 5 pounds or more seeds.

Containers of plant seeds may be any container available in the art. By way of non-limiting example, a container may be a box, a bag, a packet, a pouch, a tape roll, a pail, a foil, or a tube.

Seeds contained in a containers may be treated or untreated seeds. For example, the seeds can be treated to improve germination, for example, by priming the seeds, or by disinfection to protect against seed-borne pathogens. In another example, seeds can be coated with any available coating to improve, for example, plantability, seed emergence, and protection against seed-borne pathogens. Seed coating can be any form of seed coating including, but not limited to pelleting, film coating, and encrustments.

At least 10% of seeds within a container may be seeds of the invention. For example, at least 10%, 20%, 30%, 40%, 50%, 60%, 70%, 80%, 90%, 95%, 99% or 100% of the seeds in the container may be seeds of the invention.

The seeds of the invention may be hybrid seeds produced by a method including crossing a first plant according to the invention, with a second plant; and obtaining seeds. For example, crossing a plant including a nucleic acid molecule or expression vector of the invention with another plant.

The term “hybrid seed” refers to a seed produced by cross-pollinating two plants. Plants grown from hybrid seeds may have improved agricultural characteristics, such as better yield, greater uniformity, and/or disease resistance. Hybrid seeds do not breed true, i.e., the seed produced by self-fertilizing a hybrid plant (the plant grown from a hybrid seed) does not reliably result the next generation in an identical hybrid plant. Therefore, new hybrid seeds must be produced from the parent plant lines for each planting. Since most crop plants have both male and female organs, hybrid seeds can only be produced by preventing self-pollination of the female parent and allowing or facilitating pollination with the desired pollen. There are a variety of methods to prevent self-pollination of the female parent, one method by which self-pollination is prevented is mechanical removal of the pollen producing organ before pollen shed. Commercial hybrid maize seed (maize, Zea mays) production typically involves planting the desired male and female parental lines, usually in separate rows or blocks in an isolated field, treating the female parent plant to prevent pollen shed, ensuring pollination of the female by only the designated male parent, and harvesting hybrid seed from only the female parent. Hybrid seeds may be the result of a single cross (e.g., a first generation cross between two inbred lines), a modified single cross (e.g., a first generation cross between two inbred lines, one or other of which may have been modified slightly by the use of closely related crossing), a double cross (e.g., a first generation of a cross between two single crosses), a three-way cross (e.g., a first generation of a cross between a single cross and an inbred line), a top cross (e.g., the first generation of a cross between an inbred line and an open-pollinated variety, or the first generation of a cross between a single-cross and an open-pollinated variety), or an open pollinated variety (e.g., a population of plants selected to a standard which may show variation but has characteristics by which a variety can be differentiated from other varieties).

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

The plants of the invention, that include a nucleic acid molecule, expression vector or HST enzyme of the invention, may be used in methods of controlling undesired vegetation in the vicinity of the plant. The methods may include applying an effective amount of at least one HST-inhibiting herbicide to the undesired vegetation and the plant.

In addition, plants of the invention, that include a nucleic acid, expression vector or HST enzyme of the invention, may be used in methods of enhancing plant growth by controlling undesired vegetation in the vicinity of the plant. The methods may include applying an effective amount of at least one HST-inhibiting herbicide to the undesired vegetation and the plant.

The control of undesired vegetation is understood as meaning the killing of undesired vegetation and/or otherwise retarding or inhibiting the normal growth of the undesired vegetation. Undesired vegetation, in the broadest sense, refers to all those plants which grow in locations where they are undesired.

Undesired vegetation may include, for example, dicotyledonous and monocotyledonous weeds. Dicotyledonous weeds include, but are not limited to, weeds of the genera: Sinapis, Lepidium, Galium, Slellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera.

In addition, undesired vegetation can include, for example, crop plants that are growing in an undesired location. For example, a volunteer maize plant that is in a field that predominantly comprises soybean plants can be considered a weed, if the maize plant is undesired in the field of soybean plants.

An “effective amount” or “effective concentration” refers to an amount and concentration, respectively, of HST-inhibiting herbicides that is sufficient to kill or inhibit the growth of a similar, wild-type, plant, plant tissue, plant cell, microspore, or host cell, but that said amount does not kill or inhibit as severely the growth of the at least partially resistant HST-inhibiting herbicide plants, parts thereof, plant tissues, plant cells, and seeds of the invention. Typically, the effective amount of an herbicide is an amount that is routinely used in agricultural production systems to kill unwanted vegetation of interest. Such an amount is known to those of ordinary skill in the art, or can be easily determined using methods known in the art. Furthermore, it is recognized that the effective amount of an herbicide in an agricultural production system might be substantially different than an effective amount of an herbicide for a plant culture system such as, for example, the microspore culture system. An effective amount may be at least 10 grams of active compound per hectare (g ai/ha). For example an HST-inhibiting herbicide may be applied at a concentration of at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500 grams of active compound per hectare.

It should be understood that in the aforementioned methods the HST-inhibiting herbicides may be applied to the vicinity of the plant pre-emergence of the crop and/or post-emergence of the crop—a so-called “over-the-top” application.

“Preemergent” refers to an herbicide which is applied to the vicinity of an at least partially HST-inhibiting herbicide resistant plant of the invention (e.g., a field or area of cultivation) before the plant emerges visibly from the soil and/or before germination of a seed. “Postemergent” refers to an herbicide which is applied to the vicinity of an at least partially HST-inhibiting herbicide resistant plant of the invention after a plant emerges visibly from the soil. In some instances, the terms “preemergent” and “postemergent” are used with reference to a weed or undesired vegetation in the vicinity of an at least partially HST-inhibiting herbicide resistant plant of the invention, and in some instances these terms are used with reference to a crop plant in the vicinity of an at least partially HST-inhibiting herbicide resistant plant of the invention. When used with reference to a weed or undesired vegetation, these terms may apply to only a particular type of weed or species of weed or undesired vegetation that is present or believed to be present in the area of interest. While any herbicide may be applied in a preemergent and/or postemergent treatment, some herbicides are known to be more effective in controlling a weed or weeds or undesired plants when applied either preemergence or postemergence. HST-inhibiting herbicides may be applied “preplant incorporation” which involves the incorporation of HST-inhibiting herbicides into the soil prior to planting.

The rates of application of an HST-inhibiting herbicide may vary within wide limits and depend on the nature of the soil, the method of application (pre-emergence; post-emergence; application to the seed furrow; no tillage application etc.), the plant, the undesired vegetation to be controlled, the prevailing climatic conditions, and other factors governed by the method of application, the time of application and the target plant. The HST-inhibiting herbicide may be applied at a rate of at least 10 L/ha. For example at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000 L/ha. In some examples, the HST-inhibiting herbicide may be applied at a rate of 200 L/ha.

The application is generally made by spraying the HST-inhibiting herbicide, typically by tractor mounted sprayer for large areas, but other methods such as dusting (for powders), drip or drench can also be used.

According to the present invention, “enhancing plant growth of a plant” means an improvement in plant vigour, an improvement in plant quality, improved tolerance to stress factors, and/or improved input use efficiency.

An ‘improvement in plant vigour’ means that certain traits are improved qualitatively or quantitatively when compared with the same trait in a control plant (such as a plant not including an at least partially HST-inhibiting herbicide resistant HST enzyme as described herein) which has been exposed to an HST-inhibiting herbicide and grown under the same conditions in the absence of the a nucleic acid, expression vector or HST enzyme of the invention. Such traits include, but are not limited to, early and/or improved germination, improved emergence, the ability to use less seeds, increased root growth, a more developed root system, increased root nodulation, increased shoot growth, increased tillering, stronger tillers, more productive tillers, increased or improved plant stand, less plant verse (lodging), an increase and/or improvement in plant height, an increase in plant weight (fresh or dry), bigger leaf blades, greener leaf colour, increased pigment content, increased photosynthetic activity, earlier flowering, longer panicles, early grain maturity, increased seed, fruit or pod size, increased pod or ear number, increased seed number per pod or ear, increased seed mass, enhanced seed filling, less dead basal leaves, delay of senescence, improved vitality of the plant, increased levels of amino acids in storage tissues and/or less inputs needed (e.g. less fertiliser, water and/or labour needed). A plant with improved vigour may have an increase in any of the aforementioned traits or any combination or two or more of the aforementioned traits.

An “improvement in plant quality” means that certain traits are improved qualitatively or quantitatively when compared with the same trait in a control plant (such as a plant not including an at least partially HST-inhibiting herbicide resistant HST enzyme as described herein) which has been exposed to an HST-inhibiting herbicide and under the same conditions. Such traits include, but are not limited to, improved visual appearance of the plant, reduced ethylene (reduced production and/or inhibition of reception), improved quality of harvested material, e.g. seeds, fruits, leaves, vegetables (such improved quality may manifest as improved visual appearance of the harvested material), improved carbohydrate content (e.g. increased quantities of sugar and/or starch, improved sugar acid ratio, reduction of reducing sugars, increased rate of development of sugar), improved protein content, improved oil content and composition, improved nutritional value, reduction in anti-nutritional compounds, improved organoleptic properties (e.g. improved taste) and/or improved consumer health benefits (e.g. increased levels of vitamins and anti-oxidants)), improved post-harvest characteristics (e.g. enhanced shelf-life and/or storage stability, easier processability, easier extraction of compounds), more homogenous crop development (e.g. synchronised germination, flowering and/or fruiting of plants), and/or improved seed quality (e.g. for use in following seasons). A plant with improved quality may have an increase in any of the aforementioned traits or any combination or two or more of the aforementioned traits.

An “improved tolerance to stress factors” means that certain traits are improved qualitatively or quantitatively when compared with the same trait in a control plant (such as a plant not including an at least partially HST-herbicide resistant HST enzyme as described herein) which has been exposed to an HST-inhibiting herbicide and grown under the same conditions. Such traits include, but are not limited to, an increased tolerance and/or resistance to biotic and/or abiotic stress factors, and in particular abiotic stress factors which cause sub-optimal growing conditions such as drought (e.g. any stress which leads to a lack of water content in plants, a lack of water uptake potential or a reduction in the water supply to plants), cold exposure, heat exposure, osmotic stress, UV stress, flooding, increased salinity (e.g. in the soil), increased mineral exposure, ozone exposure, high light exposure and/or limited availability of nutrients (e.g. nitrogen and/or phosphorus nutrients). A plant with improved tolerance to stress factors may have an increase in any of the aforementioned traits or any combination or two or more of the aforementioned traits. In the case of drought and nutrient stress, such improved tolerances may be due to, for example, more efficient uptake, use or retention of water and nutrients. In particular, the compounds or compositions of the present invention are useful to improve tolerance to drought stress.

An “improved input use efficiency” means that the plants are able to grow more effectively using given levels of inputs compared to the growth of control plants (such as a plant not including an at least partially HST-inhibiting herbicide resistant HST enzyme as described herein) which has been exposed to an HST-inhibiting herbicide and grown under the same conditions. In particular, the inputs include, but are not limited to fertiliser (such as nitrogen, phosphorous, potassium, micronutrients), light and water. A plant with improved input use efficiency may have an improved use of any of the aforementioned inputs or any combination of two or more of the aforementioned inputs.

Other effects of regulating or improving the growth of a plant may include a decrease in plant height, or reduction in tillering, which are beneficial features in plants such as crops or conditions where it is desirable to have less biomass and fewer tillers.

Any or all of the above plant enhancements may lead to an improved yield by improving e.g. plant physiology, plant growth and development and/or plant architecture. In the context of the present invention ‘yield’ includes, but is not limited to, (i) an increase in biomass production, grain yield, starch content, oil content and/or protein content, which may result from (a) an increase in the amount produced by the plant per se or (b) an improved ability to harvest plant matter, (ii) an improvement in the composition of the harvested material (e.g. improved sugar acid ratios, improved oil composition, increased nutritional value, reduction of anti-nutritional compounds, increased consumer health benefits) and/or (iii) an increased/facilitated ability to harvest the plant, improved processability of the plant and/or better storage stability/shelf life. Increased yield of an agricultural plant means that, where it is possible to take a quantitative measurement, the yield of a product of the respective plant is increased by a measurable amount over the yield of the same product of the plant produced under the same conditions, but not including an at least partially HST-herbicide resistant HST enzyme as described herein. The yield may be increased by at least 0.5%, at least 1%, at least 2%, at least 4%, at least 5% or more.

Any or all of the above plant enhancements may also lead to an improved utilisation of land, i.e. land which was previously unavailable or sub-optimal for cultivation may become available.

### EXAMPLES

### Example 1—Generation of Chlorella fusca Lines Resistant to HST Inhibitor B5U

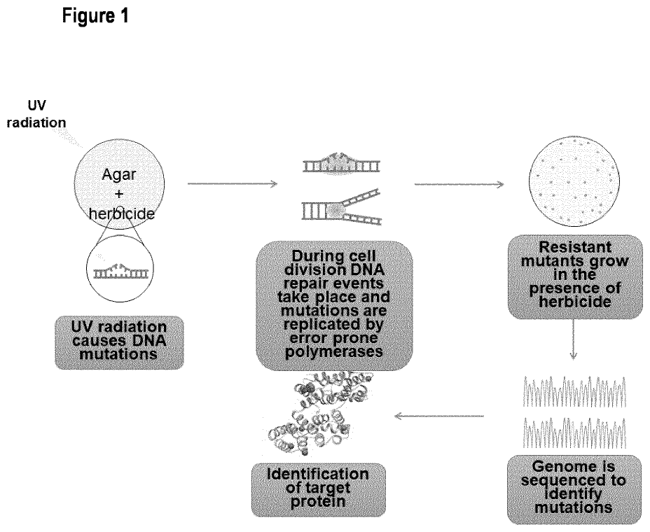

A UV mutagenesis screen was carried out with Chlorella fusca and the HST inhibitor B5u. A resulting 21 Chlorella strains were selected for further characterization from the 97 colonies surviving the herbicide treatment. A schematic summary of the experimental protocol is shown in FIG. 1. The strain ID numbers for the 21 strains selected for further characterization are shown in Table 1 below.

TABLE 1

Strain ID
Cf-496-2
Cf-496-6
Cf-496-8
Cf-496-13
Cf-496-15
Cf-496-19
Cf-496-21
Cf-496-28
Cf-496-35
Cf-496-47
Cf-496-51
Cf-496-54
Cf-496-65
Cf-496-75
Cf-496-78
Cf-496-82
Cf-496-85
Cf-496-88
Cf-496-90
Cf-496-94
Cf-496-97

Dose response tests with B5U were set-up in 96-well, flat bottomed plates. 200 μl medium was used in each well. 2 μl of 6 day old culture was added to each well, then 2 μl of DMSO or dilution of test compound were added to respective wells. 2-fold dilutions starting at 100 ppm were used per well. Plates were incubated at 25° C., 50 μmol/m²/s, 16 hour photoperiod.

Table 2 below shows the results for the lethal dose (LD) in parts per million (ppm) for each strain tested. This was assessed by a visual inspection of growth in the 96-well plate. The inhibitor concentration at which no growth is observed is then defined as the LD.

TABLE 2
resistance to HST inhibiting herbicide B5u

| Strain ID | LD ppm |
|---|---|
| Cf WT | 6.25 |
| Cf-496-2 | 6.25 |
| Cf-496-6 | >100 |
| Cf-496-8 | >100 |
| Cf-496-13 | 6.25 |
| Cf-496-15 | >100 |
| Cf-496-19 | >100 |
| Cf-496-21 | >100 |
| Cf-496-28 | >100 |
| Cf-496-35 | >100 |
| Cf-496-47 | >100 |
| Cf-496-51 | >100 |
| Cf-496-54 | >100 |
| Cf-496-65 | >100 |
| Cf-496-75 | 6.25 |
| Cf-496-78 | >100 |
| Cf-496-82 | >100 |
| Cf-496-85 | >100 |
| Cf-496-88 | >100 |
| Cf-496-90 | >100 |
| Cf-496-94 | >100 |
| Cf-496-97 | >100 |

Table 2 shows that most of the strains are resistant at 100 ppm. The resistance factor is defined as the fold difference in inhibitor concentration required to prevent growth of the resistant strains versus the parental strain. In the case of table 2 there is no control of the resistant strains at the top rate of inhibitor tested (100 ppm) and therefore the resistance factor is at least 16-fold but is potentially much higher. The genomes of the tested strains were extracted, the HST gene was amplified by PCR and sequenced. The results of the sequencing are shown in Table 3.

TABLE 3
Sequencing results showing nucleic acid
and corresponding amino acid mutations

| Strain | DNA Mutation | Amino acid change |
|---|---|---|
| Cf-496-8 | ATG → ATT & TTT → ATT | M278L & F279I |
| Cf-496-15 | AAC → AAT | T277N |
| Cf-496-19 | TTT → ATT | F275I |
| Cf-496-28 | GTG → GCG | V276A |
| Cf-496-51 | TTT → ATT | F275I |
| Cf-496-78 | TTT → ATT | F275I |
| Cf-496-94 | TTT → TTG | F275L |

Dose response tests were set up with the mutant shown in Table 4 below. Starting dose was 100 ppm and decreasing with 2-fold dilutions with C. fusca wt, the five HST mutants and the 10 compounds to which the wt is sensitive. The compounds tested are listed in Table 5 below. Plates were incubated at 25° C., 50 μmol/m2/s, 16 hour photoperiod.

TABLE 4
mutants used for dose response tests

| Cf HST mutant | Mutation |
|---|---|
| Cf-496-8 | M278L_F279I |
| Cf-496-15 | T277N |
| Cf-496-19 | F275I |
| Cf-496-28 | V276A |
| Cf-496-94 | F275L |

TABLE 5
results of dose response to HST inhibiting compounds

| CSN | Cf WT | Cf-496-8 | Cf-496-15 | Cf-496-19 | Cf-496-28 | Cf-496-94 |
|---|---|---|---|---|---|---|
| B5p | 25 | >100 | >100 | >100 | >100 | >100 |
| B5m | 12.5 | >100 | >100 | >100 | >100 | >100 |

The mutations found in the Chlorella fusca HST gene are shown to confer resistance to B5u, also confer resistance to the other HST inhibiting compounds. Compound B5p is [5-[3-chloro-6-fluoro-2-[2-(4-fluorophenyl)ethyl]phenyl]-1,3-dimethyl-6-oxo-pyridazin-4-yl]2-methylpropanoate. Compound B5m is 4-[3-chloro-6-fluoro-2-[2-[2-(trifluoromethyl)phenyl]ethyl]phenyl]-5-hydroxy-2,6-dimethyl-pyridazin-3-one.

### Example 2—HST Sequences and Expression in Tobacco Plants

Arabidopsis HST or orthologues of this (see full length HST sequences including chloroplast transit peptides), for example SEQ ID NOs: 27 to 31 expressed in transgenic tobacco. DNA sequences that encode these polypeptides (optimized for tobacco or, optionally, codon optimized according to a target crop such as soybean) are prepared synthetically. Each sequence is designed to include a 5′ fusion with TMV omega 5′ leader sequence and such that they are flanked at the 5′ end with Xhol and at the 3′ end with Kpnl to facilitate direct cloning into a suitable binary vector for Agrobacterium-based plant transformation.

In one example, the expression cassette, comprising the TMV omega 5′ leader and a HST encoding gene of interest is excised using Xhol/Kpnl and cloned into similarly digested pBIN 19 (Bevan Nucleic Acid Res. 12:8711-8721 (1984) behind a double enhanced 35S promoter ahead of a NOS 3′ transcription terminator and then transformed into E. coli DH5 alpha competent cells. DNA recovered from the E. coli is used to transform Agrobacterium tumefaciens LBA4404, and the transformed bacteria are selected on media contain rifampicin and kanamycin. Tobacco tissue is subjected to Agrobacterium-mediated transformation using methods well described in the art or as described herein. For example, a master plate of Agrobacterium tumefaciens containing the HST expressing binary vector is used to inoculate 10 ml LB (L broth) containing 100 mg/l rifampicin plus 50 mg/l kanamycin using a single bacterial colony. This is incubated overnight at 28° C. shaking at 200 rpm. This entire overnight culture is used to inoculate a 50 ml volume of LB containing the same antibiotics. Again this is cultured overnight at 28° C. shaking at 200 rpm. The Agrobacterium cells are pelleted by centrifuging at 3000 rpm for 15 minutes and then re-suspended in MS (Murashige and Skoog) medium containing 30 g/l sucrose, pH 5.9 to an OD (600 nM)=0.6. This suspension is dispensed in 25 ml aliquots into petri dishes.

Clonally micro-propagated tobacco shoot cultures are used to excise young (not yet fully expanded) leaves. The mid rib and outer leaf margins are removed and discarded, and the remaining lamina cut into 1 cm squares. These are transferred to the Agrobacterium suspension for 20 minutes. Explants are then removed, dabbed on sterile filter paper to remove excess suspension, then transferred onto solid NBM medium (MS medium containing 30 g/l sucrose, 1 mg/l BAP (benzylaminopurine) and 0.1 mg/l NAA (napthalene acetic acid) at pH 5.9 and solidified with 8 g/l Plantagar), with the abaxial surface of each explant in contact with the medium. Approximately 7 explants are transferred per plate, which are then sealed and maintained in a lit incubator at 25° C. for a 16 hour photoperiod for 3 days.

Explants are then transferred onto NBM medium containing 100 mg/l kanamycin plus antibiotics to prevent further growth of Agrobacterium (200 mg/l timentin with 250 mg/l carbenicillin). Further subculture onto this same medium was then performed every 2 weeks.

As shoots start to regenerate from the callusing leaf explants, these are removed to Shoot elongation medium (MS medium, 30 g/l sucrose, 8 g/l Plantagar, 100 mg/l kanamycin, 200 mg/l timentin, 250 mg/l carbenicillin, pH 5.9). Stable transgenic plants readily root within 2 weeks. To provide multiple plants per event to ultimately allow more than one herbicide test per transgenic plant, all rooting shoots are micropropagated to generate 3 or more rooted clones.

Putative transgenic plants that are rooting and showing vigorous shoot growth on the medium incorporating kanamycin are analysed by PCR using primers that amplified a 500 bp fragment specific to the HST transgene of interest. Evaluation of this same primer set on untransformed tobacco showed conclusively that these primers would not amplify any sequences from the native tobacco genome.

Transformed shoots are divided into 2 or 3 clones and regenerated from kanamycin resistant callus. Shoots are rooted on MS agar containing kanamycin. Surviving rooted explants are re-rooted to provide approximately 40-50 kanamycin resistant and PCR positive events from each event.

Once rooted, plantlets are transferred from agar and potted into 50% peat, 50% John Innes Soil No. 3 with slow-release fertilizer in 3 inch round pots and left regularly watered to establish for 8-12d in the glass house. Glasshouse conditions are about 24-27° C. day; 18-21° C. night and approximately a 14h photoperiod. Humidity is adjusted to ˜65% and light levels used are up to 2000 mmol/m² at bench level.

Transgenic populations of about forty tobacco plants that comprise a gene encoding a full length HST gene (e.g. SEQ ID NO: 14 (WT HST) or 22 to 69) are thus produced. Plants are selected on the basis of similar size from each population and ELISA or Mass Western tests are carried out to monitor protein transgenic HST expression levels. The highest expressing TO lines are selected to be taken forward to self and to generate T1 seed and T2 lines and seed in the normal way. Seeds from the highest expressing lines are tested for germination on agar plates containing a range of concentrations of HST-inhibiting herbicides as taught for example herein and resistant plant lines selected as showing the least damage to root growth and morphology at the highest concentrations of herbicides. Resistant plant lines exhibit a dose response in respect of herbicidal damage by HST inhibitors that is shifted to the right in comparison with similarly grown and treated wild type and null segregant plants.

### Example 3—Assay of Herbicide Tolerance in Transgenic Tobacco Plants

In order to determine if the newly discovered mutations detailed in Example 1 confer resistance to HST inhibiting compounds when expressed in plants, GM tobacco lines are to be produced and tested with HST inhibiting compounds. Transgenic tobacco plants expressing the Arabidopsis HST gene with and without mutations were tested.

Populations of transgenic tobacco comprising 20-30 transgenic events per plant transformation constructs were generated as described in Example 2. These lines were clonally propagated and 1 clone per event was sprayed with 500, 100 or 50 g ai/ha of compound 1+0.2% Genapol X080 with a spray volume of 200 L/Ha (“g ai/ha” refers to grams of active compound per hectare). Table 6 below shows the

Herbicidal damage was visually assessed across the population and a herbicide damage score given at 7 and 14 days. A score of 0 indicates no visible damage or stunting whereas a score of 100 indicates a complete death of the plant. 20 transgenic events (i.e. individual transgenic plants) from each GM tobacco line were transplanted to soil 1 week prior to spraying with HST inhibiting compounds. WT tobacco was included as a control. Table 6 below shows the treatment regimens used.

TABLE 6
treatment regimens for testing HST inhibitor resistance

| Treatment | | Application rate | Formulation |
|---|---|---|---|
| no | Compound | (g ai/ha) | and Adjuvant |
| 1 | Bu | 500 | Genapol X080 |
| | | | 0.2% |
| 2 | B5a | 100 | Genapol X080 |
| | | | 0.2% |
| 3 | B5b | 100 | Genapol X080 |
| | | | 0.2% |
| 4 | B8a | 50 | Genapol X080 |
| | | | 0.2% |

The mutated genes are identified in Table 7 below.

TABLE 7
Plants transformed with mutant Arabidopsis thaliana
HST (or WT Arabidopsis thaliana HST)

| DNA | | | |
|---|---|---|---|
| ID | Name | Gene background | Mutation |
| D4291 | pBin TMV AraHST | Arabidopsis thaliana | n.a. |
| D4292 | pBin TMV AraHST V001 | Arabidopsis thaliana | F276I |
| D4293 | pBin TMV AraHST V002 | Arabidopsis thaliana | V277A |
| D4294 | pBin TMV AraHST V003 | Arabidopsis thaliana | T278N |
| D4295 | pBin TMV AraHST V004 | Arabidopsis thaliana | F280I |
| D4296 | pBin TMV AraHST V005 | Arabidopsis thaliana | F280L |

The results are shown in Tables 8 to 14 below. The values in Tables 8 to 14 below refer to percentage of damage to the tested plants and show the damage scores for 20 populations of plants expressing either Arabidopsis WT HST gene (SEQ ID NO. 14), the Arabidopsis HST mutated gene according to SEQ ID NO. 27, the Arabidopsis HST mutated gene according to SEQ ID NO. 28, the Arabidopsis HST mutated gene according to SEQ ID NO. 29, the Arabidopsis HST mutated gene according to SEQ ID NO. 30, or the Arabidopsis HST mutated gene according to SEQ ID NO. 31. A control population of wild-type Samsun tobacco was also assessed for herbicide damage. The averaged damage scores across the population of transgenic plants for each construct is given in Table 15. It is clear that the overexpression of Arabidopsis HST gene (SEQ ID NO.14) does not increase tolerance to herbicide B5U, B5A, B5B, and B8A. However the mutated versions of the HST gene carrying the F276I, V277A, F280I or F280L mutations (SEQ ID NOs: 27, 28, 30 or 31) clearly display increased tolerance.

As can also be seen from the results shown in Tables 8 to 14, apart from Variant 003 (V003) which has mutation T278N, all of the tested mutated proteins are more tolerant to the compounds than the unmutated (or wild type) version. Variant 002 (V002) shows the highest tolerance levels.

TABLE 8
results of percentage of damage to WT tobacco plants
WT tobacco control

| | T1 | T2 | T3 | T4 |
|---|---|---|---|---|
| | B5u | B5a | B5b | B8a |
| | (500 gai/ha) | (100 gai/ha) | (100 gai/ha) | (50 gai/ha) |
| 1 | 90 | 90 | 90 | 95 |
| 2 | 90 | 85 | 90 | 95 |
| 3 | 95 | 95 | 90 | 85 |
| 4 | 95 | 95 | 90 | 95 |
| 5 | 95 | 95 | 95 | 95 |
| 6 | 95 | 95 | 95 | 95 |
| 7 | 95 | 95 | 95 | 90 |
| 8 | 95 | 95 | 90 | 90 |

TABLE 9
results of percentage of damage to D4291 (WT Arabidopsis thaliana
HST) containing tobacco plant

| D4291 | pBin TMV AraHST | | | |
|---|---|---|---|---|
| GM | T1 | T2 | T3 | T4 |
| event | B5u | B5a | B5b | B8a |
| number | (500 gai/ha) | (100 gai/ha) | (100 gai/ha) | (50 gai/ha) |
| 4863 | 75 | 90 | 45 | 85 |
| 4871 | 50 | 85 | 50 | 85 |
| 4880 | 95 | 80 | 35 | 70 |
| 4881 | 95 | 100 | 95 | 95 |
| 4884 | 100 | 99 | 100 | 99 |
| 4888 | 97 | 98 | 95 | 98 |
| 4890 | 25 | 90 | 10 | 50 |
| 4894 | 98 | 98 | 90 | 99 |
| 4897 | 15 | 95 | 10 | 15 |
| 4907 | 100 | 100 | 100 | 100 |
| 4910 | 5 | 50 | 1 | 10 |
| 4911 | 95 | 95 | 90 | 90 |
| 4912 | 15 | 95 | 10 | 80 |
| 4913 | 99 | 100 | 95 | 99 |
| 4923 | 5 | 90 | 10 | 15 |
| 4926 | 10 | 75 | 1 | 5 |
| 4934 | 10 | 90 | 5 | 50 |
| 4937 | 15 | 85 | 20 | 45 |
| 4939 | 5 | 75 | 3 | 5 |
| 4946 | 95 | 95 | 80 | 85 |

TABLE 10
results of percentage of damage to D4292 (F276I mutant
Arabidopsis thaliana HST) containing tobacco plant

| D4292 | pBin TMV AraHST V001 | | | |
|---|---|---|---|---|
| GM | T1 | T2 | T3 | T4 |
| event | B5u | B5a | B5b | B8a |
| number | (500 gai/ha) | (100 gai/ha) | (100 gai/ha) | (50 gai/ha) |
| 4949 | 10 | 10 | 0 | 1 |
| 4959 | 10 | 30 | 0 | 5 |
| 4961 | 5 | 5 | 0 | 2 |
| 4963 | 5 | 15 | 5 | 3 |
| 4966 | 45 | 65 | 35 | 35 |
| 4967 | 10 | 5 | 0 | 0 |
| 4980 | 45 | 95 | 75 | 98 |
| 4981 | 5 | 5 | 20 | 0 |
| 4985 | 5 | 15 | 0 | 5 |
| 4988 | 5 | 5 | 0 | 0 |
| 4996 | 30 | 65 | 15 | 60 |
| 4997 | 97 | 95 | 98 | 95 |
| 5003 | 95 | 95 | 15 | 75 |
| 5004 | 5 | 20 | 0 | 2 |
| 5005 | 5 | 5 | 15 | 2 |
| 5008 | 5 | 10 | 0 | 1 |
| 5011 | 99 | 95 | 45 | 90 |
| 5019 | 15 | 90 | 40 | 35 |
| 5024 | 35 | 99 | 90 | 100 |
| 5030 | 20 | 80 | 75 | 45 |

TABLE 11
results of percentage of damage to D4293 (V277A mutant
Arabidopsis thaliana HST) containing tobacco plant

| D4293 | pBin TMV AraHST V002 | | | |
|---|---|---|---|---|
| GM | T1 | T2 | T3 | T4 |
| event | B5u | B5a | B5b | B8a |
| number | (500 gai/ha) | (100 gai/ha) | (100 gai/ha) | (50 gai/ha) |
| 5031 | 5 | 10 | 2 | 0 |
| 5039 | 2 | 3 | 0 | 0 |
| 5050 | 100 | 100 | 98 | 99 |
| 5052 | 5 | 10 | 0 | 0 |
| 5059 | 5 | 55 | 1 | 5 |
| 5061 | 5 | 45 | 1 | 5 |
| 5065 | 5 | 20 | 0 | 2 |
| 5066 | 100 | 95 | 95 | 95 |
| 5067 | 5 | 10 | 1 | 0 |
| 5074 | 8 | 5 | 0 | 0 |
| 5075 | 5 | 5 | 1 | 0 |
| 5076 | 10 | 20 | 0 | 1 |
| 5077 | 10 | 5 | 0 | 0 |
| 5078 | 10 | 5 | 0 | 1 |
| 5083 | 85 | 95 | 85 | 90 |
| 5085 | 5 | 10 | 1 | 1 |
| 5096 | 10 | 10 | 0 | 5 |
| 5097 | 5 | 10 | 0 | 5 |
| 5101 | 40 | 25 | 5 | 15 |
| 5105 | 10 | 25 | 0 | 1 |

TABLE 12
results of percentage of damage to D4294 (T278N mutant
Arabidopsis thaliana HST) containing tobacco plant

| D4294 | pBin TMV AraHST V003 | | | |
|---|---|---|---|---|
| GM | T1 | T2 | T3 | T4 |
| event | B5u | B5a | B5b | B8a |
| number | (500 gai/ha) | (100 gai/ha) | (100 gai/ha) | (50 gai/ha) |
| 5115 | 99 | 99 | 100 | 99 |
| 5118 | 100 | 100 | 100 | 100 |
| 5119 | 15 | 95 | 75 | 65 |
| 5125 | 100 | 100 | 100 | 100 |
| 5130 | 15 | 95 | 95 | 95 |
| 5131 | 15 | 95 | 95 | 90 |
| 5136 | 95 | 95 | 100 | 97 |
| 5137 | 90 | 95 | 95 | 90 |
| 5144 | 95 | 95 | 98 | 95 |
| 5150 | 90 | 95 | 95 | 95 |
| 5153 | 99 | 95 | 99 | 97 |
| 5160 | 10 | 95 | 90 | 50 |
| 5164 | 95 | 95 | 90 | 95 |
| 5170 | 95 | 95 | 90 | 90 |
| 5171 | 70 | 95 | 98 | 95 |
| 5178 | 99 | 100 | 99 | 100 |
| 5180 | 80 | 90 | 90 | 80 |
| 5183 | 75 | 99 | 99 | 75 |
| 5194 | 80 | 95 | 95 | 95 |
| 5196 | 99 | 95 | 95 | 97 |

TABLE 13
results of percentage of damage to D4295 (F280I mutant
Arabidopsis thaliana HST) containing tobacco plant

| D4295 | pBin TMV AraHST V004 | | | |
|---|---|---|---|---|
| GM | T1 | T2 | T3 | T4 |
| event | B5u | B5a | B5b | B8a |
| number | (500 gai/ha) | (100 gai/ha) | (100 gai/ha) | (50 gai/ha) |
| 5199 | 15 | 60 | 5 | 10 |
| 5204 | 10 | 55 | 5 | 5 |
| 5206 | 5 | 60 | 5 | 5 |
| 5210 | 35 | 85 | 50 | 30 |
| 5213 | 100 | 100 | 100 | 100 |
| 5222 | 15 | 30 | 0 | 5 |
| 5230 | 10 | 30 | 5 | 5 |
| 5232 | 20 | 15 | 0 | 5 |
| 5236 | 99 | 100 | 98 | 100 |
| 5237 | 25 | 45 | 3 | 5 |
| 5243 | 10 | 45 | 3 | 5 |
| 5244 | 99 | 100 | 95 | 95 |
| 5247 | 95 | 95 | 90 | 90 |
| 5254 | 100 | 99 | 98 | 98 |
| 5255 | 10 | 50 | 5 | 10 |
| 5256 | 10 | 40 | 0 | 5 |
| 5260 | 5 | 45 | 5 | 5 |
| 5261 | 10 | 45 | 3 | 1 |
| 5268 | 20 | 40 | 5 | 5 |
| 5276 | 15 | 65 | 10 | 10 |

TABLE 14
results of percentage of damage to D4295 (F280L mutant
Arabidopsis thaliana HST) containing tobacco plant

| D4296 | pBin TMV AraHST V005 | | | |
|---|---|---|---|---|
| GM | T1 | T2 | T3 | T4 |
| event | B5u | B5a | B5b | B8a |
| number | (500 gai/ha) | (100 gai/ha) | (100 gai/ha) | (50 gai/ha) |
| 5284 | 15 | 15 | 2 | 3 |
| 5286 | 100 | 100 | 100 | 100 |
| 5288 | 95 | 100 | 99 | 97 |
| 5292 | 20 | 10 | 0 | 3 |
| 5296 | 5 | 25 | 0 | 0 |
| 5297 | 10 | 20 | 0 | 0 |
| 5310 | 5 | 10 | 0 | 0 |
| 5312 | 15 | 10 | 0 | 0 |
| 5313 | 65 | 30 | 90 | 90 |
| 5321 | 65 | 95 | 90 | 85 |
| 5325 | 20 | 20 | 0 | 0 |
| 5332 | 60 | 80 | 60 | 70 |
| 5336 | 15 | 15 | 0 | 0 |
| 5343 | 95 | 98 | 80 | 96 |
| 5351 | 99 | 95 | 85 | 95 |
| 5352 | 10 | 20 | 0 | 0 |
| 5356 | 75 | 95 | 70 | 90 |
| 5360 | 30 | 45 | 10 | 10 |
| 5361 | 100 | 90 | 95 | 95 |
| 5362 | 100 | 100 | 100 | 100 |

TABLE 15
Average percentage of damage

| | | Average percentage damage | | | | |
|---|---|---|---|---|---|---|
| | | T1 | T2 | T3 | T4 | |
| DNA | | B5u | B5a | B5b | B8a | Overall |
| ID | Mutation | (500 gai/ha) | (100 gai/ha) | (100 gai/ha) | (50 gai/ha) | Average |
| D4291 | n.a. | 55 | 89 | 47 | 64 | 64 |
| D4292 | F276I | 28 | 45 | 26 | 33 | 33 |
| D4293 | V277A | 22 | 28 | 15 | 16 | 20 |
| D4294 | T278N | 76 | 96 | 95 | 90 | 89 |
| D4295 | F280I | 35 | 60 | 29 | 30 | 39 |
| D4296 | F280L | 50 | 54 | 44 | 47 | 49 |

### Example 4—Assay of Herbicide Tolerance of Rice (Oryza sativa) HST Variants in Transgenic Tobacco Plants

In order to determine if the newly discovered mutations detailed in Example 1 confer resistance to HST inhibiting compounds when expressed in plants, GM tobacco lines were produced and tested with HST inhibiting compounds. Transgenic tobacco plants expressing the Oryza sativa HST gene with and without mutations were tested.

Populations of transgenic tobacco comprising 20-30 transgenic events per plant transformation constructs were generated as described in Example 2. These lines were clonally propagated and 1 clone per event was sprayed with 500, 100 or 50 g ai/ha of compound 1+0.2% (w/v) Genapol X080 with a spray volume of 200 L/Ha. (“g ai/ha” refers to grams of active compound per hectare). Table 15 below shows the treatment regimens used.

TABLE 15
treatment regimens for testing HST inhibitor
resistance of Oryza sativa HST variants

| Treatment | | Application rate | Formulation |
|---|---|---|---|
| No. | Compound | (g ai/ha) | and Adjuvant |
| 1 | B5u | 500 | Genapol X080 |
| | | | 0.2% |
| 2 | B5a | 100 | Genapol X080 |
| | | | 0.2% |
| 3 | B5b | 100 | Genapol X080 |
| | | | 0.2% |
| 4 | B8a | 50 | Genapol X080 |
| | | | 0.2% |

Herbicidal damage was visually assessed across the population and a herbicide damage score given at 7 and 14 days. A score of 0 indicates no visible damage or stunting whereas a score of 100 indicates a complete death of the plant. 20 transgenic events (i.e. individual transgenic plants) from each GM tobacco line were transplanted to soil 1 week prior to spraying with HST inhibiting compounds. WT tobacco was included as a control. The mutated genes are identified in Table 16 below.

TABLE 16
Plants transformed with mutant Arabidopsis thaliana
HST (or WT Arabidopsis thaliana HST)

| | | | | Corre- |
|---|---|---|---|---|
| DNA | | Gene | Muta- | sponding |
| ID | Name | background | tion | SED ID NO |
| D4424 | pBin TMV OsHST | Oryza sativa | n.a. | 18 |
| D4425 | pBin TMV OsHST V001 | Oryza sativa | F262I | 47 |
| D4426 | pBin TMV OsHST V002 | Oryza sativa | V263A | 48 |
| D4427 | pBin TMV OsHST V003 | Oryza sativa | T264N | 49 |
| D4428 | pBin TMV OsHST V004 | Oryza sativa | F266I | 50 |
| D4429 | pBin TMV OsHST V005 | Oryza sativa | F266L | 51 |

The results are shown in Tables 17 to 23 below. The values in Tables 17 to 23 below refer to percentage of damage to the tested plants and show the damage scores for 20 populations of plants expressing either Oryza sativa WT HST gene (SEQ ID NO. 18), the Oryza sativa HST mutated gene according to SEQ ID NO. 47, the Oryza sativa HST mutated gene according to SEQ ID NO. 48, the Oryza sativa HST mutated gene according to SEQ ID NO. 49, the Oryza sativa HST mutated gene according to SEQ ID NO. 50, or the Oryza sativa HST mutated gene according to SEQ ID NO. 51. A control population of wild-type Samsun tobacco was also assessed for herbicide damage. The averaged damage scores across the population of transgenic plants for each construct is given in Table 24. It is clear that the overexpression of Oryza sativa HST gene (SEQ ID NO.18) does not increase tolerance to herbicide B5U, B5A, B5B, and B8A. However the mutated versions of the HST gene carrying the F262I, V263A, F26661, or F266L mutations (SEQ ID NOs: 47, 48, 50 or 51) clearly display increased tolerance.

TABLE 17
results of percentage of damage to WT tobacco plants

| WT | T1 | T2 | T3 | T4 |
|---|---|---|---|---|
| Event | B5u | B5a | B5b | B8a |
| No. | (500 gai/ha) | (100 gai/ha) | (100 gai/ha) | (50 gai/ha) |
| 1 | 95 | 95 | 93 | 95 |
| 2 | 95 | 95 | 90 | 95 |
| 3 | 96 | 95 | 97 | 96 |
| 4 | 98 | 90 | 95 | 95 |
| 5 | 98 | 95 | 96 | 96 |
| 6 | 97 | 95 | 95 | 95 |
| 7 | 96 | 95 | 95 | 95 |
| 8 | 95 | 95 | 97 | 95 |

TABLE 18
results of percentage of damage to D4424 (WT
Oryza sativa HST) containing tobacco plant

| | pBin TMV OsHST | | | |
|---|---|---|---|---|
| D4424 | T1 | T2 | T3 | T4 |
| Event | B5u | B5a | B5b | B8a |
| No. | (500 gai/ha) | (100 gai/ha) | (100 gai/ha) | (50 gai/ha) |
| 6544 | 25 | 95 | 85 | 93 |
| 6545 | 10 | 90 | 85 | 3 |
| 6549 | 45 | 96 | 85 | 10 |
| 6552 | 70 | 95 | 85 | 5 |
| 6553 | 10 | 95 | 85 | 5 |
| 6556 | 10 | 90 | 45 | 5 |
| 6561 | 15 | 95 | 65 | 40 |
| 6566 | 90 | 95 | 95 | 95 |
| 6570 | 15 | 97 | 90 | 60 |
| 6574 | 15 | 98 | 90 | 3 |
| 6575 | 15 | 97 | 75 | 5 |
| 6576 | 10 | 95 | 60 | 5 |
| 6578 | 30 | 90 | 80 | 35 |
| 6587 | 15 | 96 | 98 | 10 |
| 6590 | 95 | 90 | 95 | 95 |
| 6594 | 20 | 96 | 95 | 35 |
| 6598 | 15 | 85 | 80 | 85 |
| 6600 | 15 | 95 | 70 | 10 |
| 6612 | 80 | 98 | 98 | 90 |
| 6618 | 25 | 80 | 70 | 35 |

TABLE 19
results of percentage of damage to D4425 (F262I mutant
Oryza sativa HST) containing tobacco plant

| | pBin TMV OsHST V001 | | | |
|---|---|---|---|---|
| D4425 | T1 | T2 | T3 | T4 |
| Event | B5u | B5a | B5b | B8a |
| No. | (500 gai/ha) | (100 gai/ha) | (100 gai/ha) | (50 gai/ha) |
| 6628 | 15 | 65 | 25 | 10 |
| 6633 | 25 | 65 | 15 | 5 |
| 6637 | 40 | 70 | 50 | 15 |
| 6640 | 60 | 90 | 40 | 80 |
| 6644 | 15 | 50 | 5 | 3 |
| 6645 | 30 | 55 | 3 | 3 |
| 6646 | 25 | 95 | 80 | 65 |
| 6650 | 20 | 80 | 5 | 5 |
| 6653 | 15 | 40 | 10 | 25 |
| 6668 | 50 | 90 | 65 | 10 |
| 6669 | 30 | 90 | 75 | 20 |
| 6673 | 55 | 95 | 85 | 45 |
| 6675 | 15 | 60 | 5 | 5 |
| 6677 | 25 | 70 | 3 | 0 |
| 6685 | 15 | 20 | 0 | 5 |
| 6689 | 40 | 60 | 15 | 40 |
| 6690 | 20 | 40 | 5 | 5 |
| 6692 | 90 | 95 | 85 | 95 |
| 6694 | 80 | 55 | 65 | 40 |
| 6698 | 85 | 90 | 80 | 75 |

TABLE 20
results of percentage of damage to D4426 (V263A mutant
Oryza sativa HST) containing tobacco plant

| | pBin TMV OsHST V002 | | | |
|---|---|---|---|---|
| D4426 | T1 | T2 | T3 | T4 |
| Event | B5u | B5a | B5b | B8a |
| No. | (500 gai/ha) | (100 gai/ha) | (100 gai/ha) | (50 gai/ha) |
| 6735 | 15 | 45 | 0 | 5 |
| 6740 | 45 | 0 | 5 | 0 |
| 6751 | 15 | 10 | 0 | 0 |
| 6777 | 35 | 15 | 10 | 65 |
| 6784 | 10 | 40 | 3 | 0 |

TABLE 21
results of percentage of damage to D4427 (T264N mutant
Oryza sativa HST) containing tobacco plant

| | pBin TMV OsHST V003 | | | |
|---|---|---|---|---|
| D4427 | T1 | T2 | T3 | T4 |
| Event | B5u | B5a | B5b | B8a |
| No. | (500 gai/ha) | (100 gai/ha) | (100 gai/ha) | (50 gai/ha) |
| 6795 | 90 | 95 | 95 | 60 |
| 6797 | 30 | 95 | 96 | 75 |
| 6800 | 35 | 95 | 98 | 85 |
| 6808 | 35 | 98 | 95 | 40 |
| 6817 | 45 | 80 | 97 | 5 |
| 6819 | 65 | 97 | 95 | 20 |
| 6820 | 45 | 96 | 95 | 20 |
| 6828 | 65 | 95 | 98 | 45 |
| 6838 | 55 | 98 | 96 | 10 |
| 6839 | 80 | 95 | 96 | 95 |
| 6842 | 97 | 95 | 95 | 96 |
| 6857 | 95 | 98 | 99 | 45 |
| 6860 | 50 | 99 | 98 | 35 |
| 6861 | 50 | 98 | 96 | 5 |
| 6862 | 70 | 95 | 95 | 35 |
| 6863 | 95 | 96 | 96 | 95 |
| 6864 | 70 | 98 | 90 | 30 |
| 6875 | 85 | 98 | 95 | 20 |
| 6877 | 98 | 100 | 95 | 70 |
| 6878 | 75 | 95 | 96 | 30 |

TABLE 22
results of percentage of damage to D4428 (F266I mutant
Oryza sativa HST) containing tobacco plant

| | pBin TMV OsHST V004 | | | |
|---|---|---|---|---|
| D4428 | T1 | T2 | T3 | T4 |
| Event | B5u | B5a | B5b | B8a |
| No. | (500 gai/ha) | (100 gai/ha) | (100 gai/ha) | (50 gai/ha) |
| 6880 | 45 | 80 | 3 | 0 |
| 6881 | 85 | 90 | 25 | 60 |
| 6884 | 60 | 80 | 45 | 5 |
| 6885 | 85 | 90 | 85 | 85 |
| 6888 | 55 | 80 | 15 | 5 |
| 6892 | 35 | 75 | 2 | 0 |
| 6897 | 50 | 75 | 5 | 5 |
| 6898 | 45 | 90 | 30 | 0 |
| 6902 | 80 | 85 | 65 | 25 |
| 6904 | 80 | 90 | 90 | 35 |
| 6910 | 10 | 10 | 5 | 10 |
| 6911 | 35 | 70 | 0 | 0 |
| 6920 | 35 | 60 | 0 | 0 |
| 6921 | 40 | 60 | 3 | 0 |
| 6923 | 25 | 55 | 10 | 5 |
| 6927 | 96 | 95 | 95 | 97 |
| 6928 | 45 | 75 | 0 | 10 |
| 6934 | 20 | 40 | 3 | 0 |
| 6941 | 95 | 95 | 96 | 96 |
| 6957 | 98 | 95 | 97 | 96 |

TABLE 23
results of percentage of damage to D4429 (F266L mutant
Oryza sativa HST) containing tobacco plant

| | pBin TMV OsHST V005 | | | |
|---|---|---|---|---|
| D4429 | T1 | T2 | T3 | T4 |
| Event | B5u | B5a | B5b | B8a |
| No. | (500 gai/ha) | (100 gai/ha) | (100 gai/ha) | (50 gai/ha) |
| 6963 | 20 | 2 | 0 | 0 |
| 6965 | 60 | 30 | 5 | 0 |
| 6969 | 85 | 90 | 70 | 85 |
| 6970 | 30 | 35 | 0 | 5 |
| 6973 | 15 | 10 | 0 | 0 |
| 6980 | 75 | 75 | 0 | 50 |
| 6985 | 15 | 10 | 3 | 0 |
| 6987 | 15 | 50 | 0 | 5 |
| 6988 | 10 | 3 | 0 | 0 |
| 6992 | 80 | 85 | 40 | 60 |
| 6997 | 70 | 60 | 8 | 40 |
| 6998 | 65 | 80 | 60 | 50 |
| 7012 | 70 | 90 | 45 | 65 |
| 7022 | 20 | 5 | 5 | 40 |
| 7025 | 20 | 20 | 0 | 5 |
| 7031 | 60 | 80 | 70 | 35 |
| 7032 | 20 | 3 | 0 | 0 |
| 7035 | 10 | 3 | 0 | 0 |
| 7040 | 50 | 70 | 50 | 0 |
| 7043 | 10 | 45 | 0 | 5 |

TABLE 24
Average percentage of damage for Oryza sativa HST variants and wild type HST

| | | Average percentage damage | | | | |
|---|---|---|---|---|---|---|
| | | T1 | T2 | T3 | T4 | |
| DNA | | B5u | B5a | B5b | B8a | Overall |
| ID | Mutation | (500 gai/ha) | (100 gai/ha) | (100 gai/ha) | (50 gai/ha) | Average |
| D4424 | n.a. | 31 | 93 | 82 | 36 | 61 |
| D4425 | F262I | 38 | 69 | 36 | 28 | 42 |
| D4426 | V263A | 24 | 22 | 4 | 14 | 16 |
| D4427 | T264N | 67 | 96 | 96 | 46 | 76 |
| D4428 | F266I | 56 | 75 | 34 | 27 | 48 |
| D4429 | F266L | 40 | 42 | 18 | 22 | 31 |

As can also be seen from the results shown in Tables 17 to 23, apart from Variant 003 (V003) which has mutation T264N, all of the tested mutated proteins are more tolerant to the compounds than the unmutated (or wild type) version. Variant 002 (V002) had fewer events compared to wild type plants or wild type Oryza sativa HST. Variant 005 (V005) shows the highest tolerance levels.

1. A homogentisate solanesyltransferase (HST) enzyme or an active fragment thereof, comprising the amino acid sequence motif: F[V/M]TX[F/Y](SEQ ID NO: 1), wherein X is any amino acid; and wherein one or more of the amino acid residues of the motif are mutated.

2. An HST enzyme or active fragment thereof as claimed in claim 1, wherein X is a neutral amino acid or an hydrophobic amino acid; preferably wherein X is an amino acid selected from leucine, methionine, phenylalanine, isoleucine, valine, tyrosine or cysteine.

3. An HST enzyme or active fragment thereof as claimed in claim 1, wherein the mutation at:
(a) position 1 of the motif comprises a substitution; preferably a non-conservative substitution; more preferably a substitution with an aliphatic amino acid; and/or
(b) position 2 of the motif comprises a substitution; preferably a conservative substitution; more preferably a substitution with an aliphatic amino acid; and/or
(c) position 3 of the motif comprises a substitution; preferably a non-conservative substitution; more preferably a substitution with an acidic amino acid; and/or
(d) position 5 of the motif comprises a substitution; preferably a non-conservative substitution; more preferably a substitution with an aliphatic amino acid.

4. An HST enzyme or active fragment thereof as claimed in claim 1, wherein
(a) position 1 of the motif is mutated to comprise isoleucine (SEQ ID NO: 2); and/or
(b) position 2 of the motif is mutated to comprise alanine (SEQ ID NO: 3); and/or
(c) position 3 of the motif is mutated to comprise isoleucine (SEQ ID NO: 4); and/or
(d) position 5 of the motif is mutated to comprise isoleucine (SEQ ID NO: 5) or lysine (SEQ ID NO: 6).

5. An HST enzyme or active fragment thereof as claimed in claim 1, wherein the motif is comprised within an amino acid sequence comprising any one of SEQ ID NOs: 13 to 21, or a sequence of at least 70% identity therewith.

6. An HST enzyme or active fragment thereof as claimed in claim 1, wherein the HST enzyme is at least partially resistant to inhibition by an HST-inhibiting compound.

7. An HST enzyme or active fragment thereof as claimed in claim 6, wherein the HST enzyme is at least 10-fold more resistant to inhibition by an HST-inhibiting compound than a control or wild-type HST enzyme not having the or each mutation; preferably wherein the control or wild-type HST has the amino acid sequence of any one of SEQ ID NO: 14, 16, 18, 20 or 21.

8. An HST enzyme or active fragment thereof as claimed in claim 1, wherein the mutation is an amino acid substitution selected from:
(a) F196I, V197A, T199N, F200I or F200L of SEQ ID NO:13 or the corresponding amino acids of any one of SEQ ID NOs: 14 to 16, or a combination thereof,
(b) F199I, V200A, T201N, F203I or F203L of SEQ ID NO:17, or the corresponding amino acids of any one of SEQ ID NO: 18, or a combination thereof, or
(c) F198I, V199A, T200N, F202I or F202L of SEQ ID NO:19, or the corresponding amino acids of any one of SEQ ID NOs: 20 to 21, or a combination thereof.

9. An HST enzyme or active fragment thereof as claimed in claim 1, wherein the HST enzyme comprises an additional amino acid sequence; preferably wherein the additional amino acid sequence is transit peptide.

10. An HST enzyme as claimed in claim 1, comprising an amino acid sequence which is at least 70% identical to a sequence selected from SEQ ID NOs: 22 to 69, or an active fragment thereof.

11. An HST enzyme as claimed in claim 1, comprising or consisting of an amino acid sequence selected from SEQ ID NO: 22 to 69 or an active fragment thereof.

12. An HST enzyme or active fragment thereof as claimed in claim 1, wherein the HST-inhibiting herbicide is selected from one or more of:
(a) 4-[3-chloro-6-fluoro-2-[2-(2-fluoro-4-pyridyl)ethyl]phenyl]-5-hydroxy-2,6-dimethyl-pyridazin-3-one (compound B5a);
(b) 4-[3-chloro-6-fluoro-2-[2-(1-methylpyrazol-4-yl)ethyl]phenyl]-5-hydroxy-2,6-dimethyl-pyridazin-3-one (compound B5b);
(c) [5-[3-chloro-6-fluoro-2-[2-[4-(trifluoromethyl)phenyl]ethyl]phenyl]-1,3-dimethyl-6-oxo-pyridazin-4-yl]2-methylpropanoate (compound B5u); and
(d) 4-[3-chloro-6-fluoro-2-[2-(2-methyl-1,3-benzoxazol-6-yl)ethyl]phenyl]-5-hydroxy-2,6-dimethyl-pyridazin-3-one (compound B8a).

13. An isolated nucleic acid comprising a nucleotide sequence encoding a homogentisate solanesyltransferase (HST) enzyme or active fragment thereof, as set forth in claim 1.

14. An expression vector comprising a nucleic acid of claim 13.

15. An expression vector as claimed in claim 14, further comprising an expression regulatory sequence or sequences; preferably wherein:
(a) the expression regulatory sequence or sequences comprise one or more of a transcription initiation region and a translation initiation region that are functional in a plant; optionally wherein the expression vector comprises a nucleic acid sequence encoding a transit peptide; and/or
(b) the expression regulatory sequences are operably linked to the nucleic acid encoding the HST enzyme or active fragment thereof.

16. A plant, plant part or plant cell comprising an HST enzyme of claim 1.

17. A plant, plant part or plant cell as claimed in claim 16, wherein the HST enzyme or active fragment thereof is actively expressed from the nucleic acid or the expression vector.

18. A plant, plant part or plant cell as claimed in claim 16, wherein the plant, part or cell has an increased resistance to an HST-inhibiting herbicide as compared to a corresponding wild type or control plant, plant part or cell; optionally wherein the plant, plant part or cell is at least 10-fold more resistant to an HST-inhibiting herbicide than a corresponding wild type or control plant, part or cell.

19. A method of controlling undesired vegetation in the vicinity of a plant according to claim 16, the method comprising applying an effective amount of at least one HST-inhibiting herbicide to the undesired vegetation and to said plant.

20. A method of enhancing growth of a plant of claim 16 by controlling undesired vegetation in the vicinity of the plant, the method comprising applying an effective amount of at least one HST-inhibiting herbicide to the undesired vegetation and to the plant.

21. A method as claimed in claim 19, wherein the effective amount of said HST-inhibiting herbicide does not substantially inhibit the growth of the plant; preferably wherein the HST-inhibiting herbicide is selected from one or more of:
(a) 4-[3-chloro-6-fluoro-2-[2-(2-fluoro-4-pyridyl)ethyl]phenyl]-5-hydroxy-2,6-dimethyl-pyridazin-3-one (compound B5a);
(b) 4-[3-chloro-6-fluoro-2-[2-(1-methylpyrazol-4-yl)ethyl]phenyl]-5-hydroxy-2,6-dimethyl-pyridazin-3-one (compound B5b);
(c) [5-[3-chloro-6-fluoro-2-[2-[4-(trifluoromethyl)phenyl]ethyl]phenyl]-1,3-dimethyl-6-oxo-pyridazin-4-yl]2-methylpropanoate (compound B5u); and
(d) 4-[3-chloro-6-fluoro-2-[2-(2-methyl-1,3-benzoxazol-6-yl)ethyl]phenyl]-5-hydroxy-2,6-dimethyl-pyridazin-3-one (compound B8a).

22. A method for conferring increased HST-inhibiting herbicide resistance to a plant, plant part or plant cell as compared to a corresponding control or wild-type plant, part or cell, comprising the expression in the plant, part or cell of an HST enzyme according to claim 1.

23. A method of producing a hybrid seed comprising crossing a first plant comprising a nucleic acid molecule according to claim 13 with a second plant; and obtaining seeds.

24. A method of modifying a plant, plant part or plant cell to increase resistance to an HST-inhibiting herbicide as compared to a corresponding control or wild-type plant, comprising transforming the plant, plant part, plant cell or protoplast with:
(a) a nucleic acid molecule of claim 13; and/or
(b) one or more nucleic acid molecules encoding a gene editing system for modifying an endogenous nucleic acid sequence of the plant encoding an HST enzyme at one or more positions to produce a nucleic acid sequence according to claim 13.

25. A method as claimed in claim 24, wherein step (b) comprises editing of the endogenous nucleic acid sequence of the plant encoding an HST enzyme; preferably wherein the gene editing system comprises a CRISPR-Cas system.


SEQUENCE LISTING (72 sequences)

SEQ ID NO: 1-6 - skipped (6 entries)

SEQ ID NO: 7 - protein, 19 aa, synthetic construct
  VARIANT 1..19
  VARIANT 1: note = X is threonine or alanine [T/A]
  VARIANT 2: note = X is any amino acid
  VARIANT 4: note = X is valine or methionine [V/M]
  VARIANT 6: note = X is any amino acid
  VARIANT 7: note = X is phenylalanine or tyrosine [F/Y]
  VARIANT 9..10
  VARIANT 12..14
  VARIANT 18: note = X is any amino acid
XXFXTXXAXXIXXXKDLXD

SEQ ID NO: 8 - protein, 19 aa, synthetic construct
  VARIANT 1..19
  VARIANT 1: note = X is threonine or alanine [T/A]
  VARIANT 2: note = X is any amino acid
  VARIANT 4: note = X is valine or methionine [V/M]
  VARIANT 6: note = X is any amino acid
  VARIANT 7: note = X is phenylalanine or tyrosine [F/Y]
  VARIANT 9..10
  VARIANT 12..14
  VARIANT 18: note = X is any amino acid
XXIXTXXAXXIXXXKDLXD

SEQ ID NO: 9 - protein, 19 aa, synthetic construct
  VARIANT 1..19
  VARIANT 1: note = X is threonine or alanine [T/A]
  VARIANT 2: note = X is any amino acid
  VARIANT 6: note = X is any amino acid
  VARIANT 7: note = X is phenylalanine or tyrosine [F/Y]
  VARIANT 9..10
  VARIANT 12..14
  VARIANT 18: note = X is any amino acid
XXFATXXAXXIXXXKDLXD

SEQ ID NO: 10 - protein, 19 aa, synthetic construct
  VARIANT 1..19
  VARIANT 1: note = X is threonine or alanine [T/A]
  VARIANT 2: note = X is any amino acid
  VARIANT 4: note = X is valine or methionine [V/M]
  VARIANT 6: note = X is any amino acid
  VARIANT 7: note = X is phenylalanine or tyrosine [F/Y]
  VARIANT 9..10
  VARIANT 12..14
  VARIANT 18: note = X is any amino acid
XXFXIXXAXXIXXXKDLXD

SEQ ID NO: 11 - protein, 19 aa, synthetic construct
  VARIANT 1..19
  VARIANT 1: note = X is threonine or alanine [T/A]
  VARIANT 2: note = X is any amino acid
  VARIANT 4: note = X is valine or methionine [V/M]
  VARIANT 6: note = X is any amino acid
  VARIANT 9..10
  VARIANT 12..14
  VARIANT 18: note = X is any amino acid
XXFXTXIAXXIXXXKDLXD

SEQ ID NO: 12 - protein, 19 aa, synthetic construct
  VARIANT 1..19
  VARIANT 1: note = X is threonine or alanine [T/A]
  VARIANT 2: note = X is any amino acid
  VARIANT 4: note = X is valine or methionine [V/M]
  VARIANT 6: note = X is any amino acid
  VARIANT 9..10
  VARIANT 12..14
  VARIANT 18: note = X is any amino acid
XXFXTXLAXXIXXXKDLXD

SEQ ID NO: 13 - protein, 313 aa, Arabidopsis thaliana
  REGION 1..313: note = Arabidopsis thaliana HST (common sequence)
VGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 14 - protein, 393 aa, Arabidopsis thaliana
  REGION 1..393: note = Arabidopsis thaliana WT HST
MELSISQSPRVRFSSLAPRFLAASHHHRPSVHLAGKFISLPRDVRFTSLSTSRMRILAVALTFKSRCVYVNYEIPKDQILVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 15 - protein, 316 aa, Arabidopsis thaliana
  REGION 1..316: note = Arabidopsis thaliana WT HST (chloroplastic without transit peptide)
ACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPF

SEQ ID NO: 16 - protein, 386 aa, Arabidopsis thaliana
  REGION 1..386: note = Arabidopsis thaliana WT HST (chloroplastic with transit peptide)
MELSISQSPRVRFSSLAPRFLAASHHHRPSVHLAGKFISLPRDVRFTSLSTSRMRSKFVSTNYRKISIRACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 17 - protein, 316 aa, Oryza sativa
  REGION 1..316: note = Oryza sativa WT HST (without transit peptide)
VCSQAGAAGPAPLSKTLSDLKDSCWRFLRPHTIRGTALGSIALVARALIENPQLINWWLVFKAFYGLVALICGNGYIVGINQIYDIRIDKVNKPYLPIAAGDLSVQTAWLLVVLFAAAGFSIVVTNFGPFITSLYCLGLFLGTIYSVPPFRLKRYPVAAFLIIATVRGFLLNFGVYYATRAALGLTFQWSSPVAFITCFVTLFALVIAITKDLPDVEGDRKYQISTLATKLGVRNIAFLGSGLLIANYVAAIAVAFLMPQAFRRTVMVPVHAALAVGIIFQTWVLEQAKYTKDAISQYYRFIWNLFYAEYIFFPLI

SEQ ID NO: 18 - protein, 379 aa, Oryza sativa
  REGION 1..379: note = Oryza sativa WT HST (with transit pepetide)
MASLASPPLPCRAAATASRSGRPAPRLLGPPPPPASPLLSSASARFPRAPCNAARWSRRDAVRVCSQAGAAGPAPLSKTLSDLKDSCWRFLRPHTIRGTALGSIALVARALIENPQLINWWLVFKAFYGLVALICGNGYIVGINQIYDIRIDKVNKPYLPIAAGDLSVQTAWLLVVLFAAAGFSIVVTNFGPFITSLYCLGLFLGTIYSVPPFRLKRYPVAAFLIIATVRGFLLNFGVYYATRAALGLTFQWSSPVAFITCFVTLFALVIAITKDLPDVEGDRKYQISTLATKLGVRNIAFLGSGLLIANYVAAIAVAFLMPQAFRRTVMVPVHAALAVGIIFQTWVLEQAKYTKDAISQYYRFIWNLFYAEYIFFPLI

SEQ ID NO: 19 - protein, 315 aa, Chlorella fusca
  REGION 1..315: note = Chlorella fusca WT HST (common sequence and without transit peptide)
SSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVTMFATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 20 - protein, 392 aa, Chlorella fusca
  REGION 1..392: note = Chlorella fusca WT HST
MLSSGQNQVQQSLGRHKSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVTMFATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 21 - protein, 378 aa, Chlorella fusca
  REGION 1..378: note = Chlorella fusca WT HST (with transit peptide)
MLRSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVTMFATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 22 - protein, 313 aa, synthetic construct
  REGION 1..313: note = Arabidopsis thaliana HST (common sequence) F196I
VGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSIVTLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 23 - protein, 313 aa, synthetic construct
  REGION 1..313: note = Arabidopsis thaliana HST (common sequence) F196I
VGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFATLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 24 - protein, 313 aa, synthetic construct
  REGION 1..313: note = Arabidopsis thaliana HST (common sequence) T198N
VGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVNLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 25 - protein, 313 aa, synthetic construct
  REGION 1..313: note = Arabidopsis thaliana HST (common sequence) F200I
VGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLIALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 26 - protein, 313 aa, synthetic construct
  REGION 1..313: note = Arabidopsis thaliana HST (common sequence) F200L
VGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLLALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 27 - protein, 393 aa, synthetic construct
  REGION 1..393: note = Arabidopsis HST F276I
MELSISQSPRVRFSSLAPRFLAASHHHRPSVHLAGKFISLPRDVRFTSLSTSRMRILAVALTFKSRCVYVNYEIPKDQILVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSIVTLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 28 - protein, 393 aa, synthetic construct
  REGION 1..393: note = Arabidopsis HST V277A
MELSISQSPRVRFSSLAPRFLAASHHHRPSVHLAGKFISLPRDVRFTSLSTSRMRILAVALTFKSRCVYVNYEIPKDQILVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFATLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 29 - protein, 393 aa, synthetic construct
  REGION 1..393: note = Arabidopsis HST T278N
MELSISQSPRVRFSSLAPRFLAASHHHRPSVHLAGKFISLPRDVRFTSLSTSRMRILAVALTFKSRCVYVNYEIPKDQILVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVNLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 30 - protein, 393 aa, synthetic construct
  REGION 1..393: note = Arabidopsis HST F280I
MELSISQSPRVRFSSLAPRFLAASHHHRPSVHLAGKFISLPRDVRFTSLSTSRMRILAVALTFKSRCVYVNYEIPKDQILVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLIALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 31 - protein, 393 aa, synthetic construct
  REGION 1..393: note = Arabidopsis HST F280L
MELSISQSPRVRFSSLAPRFLAASHHHRPSVHLAGKFISLPRDVRFTSLSTSRMRILAVALTFKSRCVYVNYEIPKDQILVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLLALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 32 - protein, 317 aa, synthetic construct
  REGION 1..317: note = Arabidopsis thaliana HST (chloroplastic without transit peptide) F200I
ACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSIVTLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 33 - protein, 317 aa, synthetic construct
  REGION 1..317: note = Arabidopsis thaliana HST (chloroplastic without transit peptide) V201A
ACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFATLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 34 - protein, 317 aa, synthetic construct
  REGION 1..317: note = Arabidopsis thaliana HST (chloroplastic without transit peptide) T202N
ACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVNLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 35 - protein, 317 aa, synthetic construct
  REGION 1..317: note = Arabidopsis thaliana HST (chloroplastic without transit peptide) F204I
ACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLIALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 36 - protein, 317 aa, synthetic construct
  REGION 1..317: note = Arabidopsis thaliana HST (chloroplastic without transit peptide) F204L
ACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLLALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 37 - protein, 386 aa, synthetic construct
  REGION 1..386: note = Arabidopsis thaliana HST (chloroplastic with transit peptide) F269I
MELSISQSPRVRFSSLAPRFLAASHHHRPSVHLAGKFISLPRDVRFTSLSTSRMRSKFVSTNYRKISIRACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSIVTLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 38 - protein, 386 aa, synthetic construct
  REGION 1..386: note = Arabidopsis thaliana HST (chloroplastic with transit peptide) V270A
MELSISQSPRVRFSSLAPRFLAASHHHRPSVHLAGKFISLPRDVRFTSLSTSRMRSKFVSTNYRKISIRACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFATLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 39 - protein, 386 aa, synthetic construct
  REGION 1..386: note = Arabidopsis thaliana HST (chloroplastic with transit peptide) T271N
MELSISQSPRVRFSSLAPRFLAASHHHRPSVHLAGKFISLPRDVRFTSLSTSRMRSKFVSTNYRKISIRACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVNLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 40 - protein, 386 aa, synthetic construct
  REGION 1..386: note = Arabidopsis thaliana HST (chloroplastic with transit peptide) F273I
MELSISQSPRVRFSSLAPRFLAASHHHRPSVHLAGKFISLPRDVRFTSLSTSRMRSKFVSTNYRKISIRACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLIALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 41 - protein, 386 aa, synthetic construct
  REGION 1..386: note = Arabidopsis thaliana HST (chloroplastic with transit peptide) F273L
MELSISQSPRVRFSSLAPRFLAASHHHRPSVHLAGKFISLPRDVRFTSLSTSRMRSKFVSTNYRKISIRACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLLALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 42 - protein, 316 aa, synthetic construct
  REGION 1..316: note = Oryza sativa HST (without transit peptide) F199I
VCSQAGAAGPAPLSKTLSDLKDSCWRFLRPHTIRGTALGSIALVARALIENPQLINWWLVFKAFYGLVALICGNGYIVGINQIYDIRIDKVNKPYLPIAAGDLSVQTAWLLVVLFAAAGFSIVVTNFGPFITSLYCLGLFLGTIYSVPPFRLKRYPVAAFLIIATVRGFLLNFGVYYATRAALGLTFQWSSPVAFITCIVTLFALVIAITKDLPDVEGDRKYQISTLATKLGVRNIAFLGSGLLIANYVAAIAVAFLMPQAFRRTVMVPVHAALAVGIIFQTWVLEQAKYTKDAISQYYRFIWNLFYAEYIFFPLI

SEQ ID NO: 43 - protein, 316 aa, synthetic construct
  REGION 1..316: note = Oryza sativa HST (without transit peptide) V200A
VCSQAGAAGPAPLSKTLSDLKDSCWRFLRPHTIRGTALGSIALVARALIENPQLINWWLVFKAFYGLVALICGNGYIVGINQIYDIRIDKVNKPYLPIAAGDLSVQTAWLLVVLFAAAGFSIVVTNFGPFITSLYCLGLFLGTIYSVPPFRLKRYPVAAFLIIATVRGFLLNFGVYYATRAALGLTFQWSSPVAFITCFATLFALVIAITKDLPDVEGDRKYQISTLATKLGVRNIAFLGSGLLIANYVAAIAVAFLMPQAFRRTVMVPVHAALAVGIIFQTWVLEQAKYTKDAISQYYRFIWNLFYAEYIFFPLI

SEQ ID NO: 44 - protein, 316 aa, synthetic construct
  REGION 1..316: note = Oryza sativa HST (without transit peptide) T201N
VCSQAGAAGPAPLSKTLSDLKDSCWRFLRPHTIRGTALGSIALVARALIENPQLINWWLVFKAFYGLVALICGNGYIVGINQIYDIRIDKVNKPYLPIAAGDLSVQTAWLLVVLFAAAGFSIVVTNFGPFITSLYCLGLFLGTIYSVPPFRLKRYPVAAFLIIATVRGFLLNFGVYYATRAALGLTFQWSSPVAFITCFVNLFALVIAITKDLPDVEGDRKYQISTLATKLGVRNIAFLGSGLLIANYVAAIAVAFLMPQAFRRTVMVPVHAALAVGIIFQTWVLEQAKYTKDAISQYYRFIWNLFYAEYIFFPLI

SEQ ID NO: 45 - protein, 316 aa, synthetic construct
  REGION 1..316: note = Oryza sativa HST (without transit peptide) F203I
VCSQAGAAGPAPLSKTLSDLKDSCWRFLRPHTIRGTALGSIALVARALIENPQLINWWLVFKAFYGLVALICGNGYIVGINQIYDIRIDKVNKPYLPIAAGDLSVQTAWLLVVLFAAAGFSIVVTNFGPFITSLYCLGLFLGTIYSVPPFRLKRYPVAAFLIIATVRGFLLNFGVYYATRAALGLTFQWSSPVAFITCFVTLIALVIAITKDLPDVEGDRKYQISTLATKLGVRNIAFLGSGLLIANYVAAIAVAFLMPQAFRRTVMVPVHAALAVGIIFQTWVLEQAKYTKDAISQYYRFIWNLFYAEYIFFPLI

SEQ ID NO: 46 - protein, 316 aa, synthetic construct
  REGION 1..316: note = Oryza sativa HST (without transit peptide) F203L
VCSQAGAAGPAPLSKTLSDLKDSCWRFLRPHTIRGTALGSIALVARALIENPQLINWWLVFKAFYGLVALICGNGYIVGINQIYDIRIDKVNKPYLPIAAGDLSVQTAWLLVVLFAAAGFSIVVTNFGPFITSLYCLGLFLGTIYSVPPFRLKRYPVAAFLIIATVRGFLLNFGVYYATRAALGLTFQWSSPVAFITCFVTLLALVIAITKDLPDVEGDRKYQISTLATKLGVRNIAFLGSGLLIANYVAAIAVAFLMPQAFRRTVMVPVHAALAVGIIFQTWVLEQAKYTKDAISQYYRFIWNLFYAEYIFFPLI

SEQ ID NO: 47 - protein, 379 aa, synthetic construct
  REGION 1..379: note = Oryza sativa HST (with transit peptide) F262I
MASLASPPLPCRAAATASRSGRPAPRLLGPPPPPASPLLSSASARFPRAPCNAARWSRRDAVRVCSQAGAAGPAPLSKTLSDLKDSCWRFLRPHTIRGTALGSIALVARALIENPQLINWWLVFKAFYGLVALICGNGYIVGINQIYDIRIDKVNKPYLPIAAGDLSVQTAWLLVVLFAAAGFSIVVTNFGPFITSLYCLGLFLGTIYSVPPFRLKRYPVAAFLIIATVRGFLLNFGVYYATRAALGLTFQWSSPVAFITCIVTLFALVIAITKDLPDVEGDRKYQISTLATKLGVRNIAFLGSGLLIANYVAAIAVAFLMPQAFRRTVMVPVHAALAVGIIFQTWVLEQAKYTKDAISQYYRFIWNLFYAEYIFFPLI

SEQ ID NO: 48 - protein, 379 aa, synthetic construct
  REGION 1..379: note = Oryza sativa HST (with transit peptide) V263A
MASLASPPLPCRAAATASRSGRPAPRLLGPPPPPASPLLSSASARFPRAPCNAARWSRRDAVRVCSQAGAAGPAPLSKTLSDLKDSCWRFLRPHTIRGTALGSIALVARALIENPQLINWWLVFKAFYGLVALICGNGYIVGINQIYDIRIDKVNKPYLPIAAGDLSVQTAWLLVVLFAAAGFSIVVTNFGPFITSLYCLGLFLGTIYSVPPFRLKRYPVAAFLIIATVRGFLLNFGVYYATRAALGLTFQWSSPVAFITCFATLFALVIAITKDLPDVEGDRKYQISTLATKLGVRNIAFLGSGLLIANYVAAIAVAFLMPQAFRRTVMVPVHAALAVGIIFQTWVLEQAKYTKDAISQYYRFIWNLFYAEYIFFPLI

SEQ ID NO: 49 - protein, 379 aa, synthetic construct
  REGION 1..379: note = Oryza sativa HST (with transit peptide) T264N
MASLASPPLPCRAAATASRSGRPAPRLLGPPPPPASPLLSSASARFPRAPCNAARWSRRDAVRVCSQAGAAGPAPLSKTLSDLKDSCWRFLRPHTIRGTALGSIALVARALIENPQLINWWLVFKAFYGLVALICGNGYIVGINQIYDIRIDKVNKPYLPIAAGDLSVQTAWLLVVLFAAAGFSIVVTNFGPFITSLYCLGLFLGTIYSVPPFRLKRYPVAAFLIIATVRGFLLNFGVYYATRAALGLTFQWSSPVAFITCFVNLFALVIAITKDLPDVEGDRKYQISTLATKLGVRNIAFLGSGLLIANYVAAIAVAFLMPQAFRRTVMVPVHAALAVGIIFQTWVLEQAKYTKDAISQYYRFIWNLFYAEYIFFPLI

SEQ ID NO: 50 - protein, 379 aa, synthetic construct
  REGION 1..379: note = Oryza sativa HST (with transit pepetide) F266I
MASLASPPLPCRAAATASRSGRPAPRLLGPPPPPASPLLSSASARFPRAPCNAARWSRRDAVRVCSQAGAAGPAPLSKTLSDLKDSCWRFLRPHTIRGTALGSIALVARALIENPQLINWWLVFKAFYGLVALICGNGYIVGINQIYDIRIDKVNKPYLPIAAGDLSVQTAWLLVVLFAAAGFSIVVTNFGPFITSLYCLGLFLGTIYSVPPFRLKRYPVAAFLIIATVRGFLLNFGVYYATRAALGLTFQWSSPVAFITCFVTLIALVIAITKDLPDVEGDRKYQISTLATKLGVRNIAFLGSGLLIANYVAAIAVAFLMPQAFRRTVMVPVHAALAVGIIFQTWVLEQAKYTKDAISQYYRFIWNLFYAEYIFFPLI

SEQ ID NO: 51 - protein, 379 aa, synthetic construct
  REGION 1..379: note = Oryza sativa HST (with transit peptide) F266L
MASLASPPLPCRAAATASRSGRPAPRLLGPPPPPASPLLSSASARFPRAPCNAARWSRRDAVRVCSQAGAAGPAPLSKTLSDLKDSCWRFLRPHTIRGTALGSIALVARALIENPQLINWWLVFKAFYGLVALICGNGYIVGINQIYDIRIDKVNKPYLPIAAGDLSVQTAWLLVVLFAAAGFSIVVTNFGPFITSLYCLGLFLGTIYSVPPFRLKRYPVAAFLIIATVRGFLLNFGVYYATRAALGLTFQWSSPVAFITCFVTLLALVIAITKDLPDVEGDRKYQISTLATKLGVRNIAFLGSGLLIANYVAAIAVAFLMPQAFRRTVMVPVHAALAVGIIFQTWVLEQAKYTKDAISQYYRFIWNLFYAEYIFFPLI

SEQ ID NO: 52 - protein, 315 aa, synthetic construct
  REGION 1..315: note = Chlorella fusca HST (common sequence and without transit peptide) F198I
SSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCIVTMFATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 53 - protein, 315 aa, synthetic construct
  REGION 1..315: note = Chlorella fusca HST (common sequence and without transit peptide) V199A
SSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFATMFATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 54 - protein, 315 aa, synthetic construct
  REGION 1..315: note = Chlorella fusca HST (common sequence and without transit peptide) T200N
SSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVNMFATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 55 - protein, 315 aa, synthetic construct
  REGION 1..315: note = Chlorella fusca HST (common sequence and without transit peptide) M201L F202I
SSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVTLIATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 56 - protein, 315 aa, synthetic construct
  REGION 1..315: note = Chlorella fusca HST (common sequence and without transit peptide) F202I
SSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVTMIATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 57 - protein, 315 aa, synthetic construct
  REGION 1..315: note = Chlorella fusca HST (common sequence and without transit peptide) F202L
SSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVTMLATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 58 - protein, 392 aa, synthetic construct
  REGION 1..392: note = Chlorella fusca HST F275I
MLSSGQNQVQQSLGRHKSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCIVTMFATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 59 - protein, 392 aa, synthetic construct
  REGION 1..392: note = Chlorella fusca HST V276A
MLSSGQNQVQQSLGRHKSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFATMFATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 60 - protein, 392 aa, synthetic construct
  REGION 1..392: note = Chlorella fusca HST T277N
MLSSGQNQVQQSLGRHKSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVNMFATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 61 - protein, 392 aa, synthetic construct
  REGION 1..392: note = Chlorella fusca HST M278L F279I
MLSSGQNQVQQSLGRHKSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVTLIATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 62 - protein, 392 aa, synthetic construct
  REGION 1..392: note = Chlorella fusca HST F279I
MLSSGQNQVQQSLGRHKSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVTMIATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 63 - protein, 392 aa, synthetic construct
  REGION 1..392: note = Chlorella fusca HST F279L
MLSSGQNQVQQSLGRHKSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVTMLATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 64 - protein, 378 aa, synthetic construct
  REGION 1..378: note = Chlorella fusca HST (with transit peptide) F261I
MLRSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCIVTMFATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 65 - protein, 378 aa, synthetic construct
  REGION 1..378: note = Chlorella fusca HST (with transit peptide) V262A
MLRSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFATMFATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 66 - protein, 378 aa, synthetic construct
  REGION 1..378: note = Chlorella fusca HST (with transit peptide) T263N
MLRSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVNMFATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 67 - protein, 378 aa, synthetic construct
  REGION 1..378: note = Chlorella fusca HST (with transit peptide) M264L F265I
MLRSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVTLIATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 68 - protein, 378 aa, synthetic construct
  REGION 1..378: note = Chlorella fusca HST (with transit peptide) F265I
MLRSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVTMIATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 69 - protein, 378 aa, synthetic construct
  REGION 1..378: note = Chlorella fusca (with transit peptide) F265L
MLRSSRWSCSHAFQPSRTSQWPRLLPGLHSKVQQQHHLHQLPQQLQQHQLQQPERLVSTQAASSSTYSGPDGWSGDGSQGSFLSAFWRFLRPHTIRGTILGTSAVVTKVVLSNMEAIDWGLLPRALMGLVALLCGNGYIVGINQIYDVDIDAVNKPFLPVAAGDMSPGTAWVLVLALAAAGLGIVATNFGNLITGLYGFGLLLGTVYSVPPLRLKRFAVPAFLIIATVRGFLLNFGVYHATRAALQLPFEWSPAILFITCFVTMLATVIAITKDLPDIEGDKANNISTFATRLGVKNVSLLGVGMLLLNYVMAGVFALKYSSYFNVPVMLGAHALLALVLILRTTKLAAAGYTQQAVISFYRWIWNLFYSEYLVYVLI

SEQ ID NO: 70 - protein, 69 aa, Arabidopsis thaliana
  REGION 1..69: note = Arabidopsis thaliana HST transit peptide
MELSISQSPRVRFSSLAPRFLAASHHHRPSVHLAGKFISLPRDVRFTSLSTSRMRSKFVSTNYRKISIR

SEQ ID NO: 71 - protein, 63 aa, Oryza sativa
  REGION 1..63: note = Oryza sativa HST transit peptide
MASLASPPLPCRAAATASRSGRPAPRLLGPPPPPASPLLSSASARFPRAPCNAARWSRRDAVR

SEQ ID NO: 72 - protein, 63 aa, Chlorella fusca
  REGION 1..63: note = Chlorella fusca transit peptide
MLRSSRWSCSHAFQPSRTSQWPRLLPGLHSKVQQQHHLHQLPQQLQQHQLQQPERLVSTQAAS